(19) United States
(12) Patent Application Publication
(10) Pub. No.: US 2016/0145219 A1
(43) Pub. Date: May 26, 2016

(54) STROBILURIN TYPE COMPOUNDS FOR COMBATING PHYTOPATHOGENIC FUNGI

(71) Applicant: BASF SE, Ludwigshafen (DE)

(72) Inventors: Christian WINTER, Ludwigshafen (DE); Joachim RHEINHEIMER, Ludwigshafen (DE); Antje WOLF, Ludwigshafen (DE); Manojkumar POONOTH, Mannheim (DE); Violeta TERTERYAN, Mannheim (DE); Christine WIEBE, Mannheim (DE); Doris KREMZOW-GRAW, Heidelberg (DE); Franz ROEHL, Goennheim (DE); Wassilios GRAMMENOS, Ludwigshafen (DE); Sebastian Georgios ROHRER, Ketsch (DE); Andy WIEJA, Ludwigshafen (DE); Claudia ROSENBAUM, Einhausen (DE)

(21) Appl. No.: 14/900,214

(22) PCT Filed: Jun. 25, 2014
(86) PCT No.: PCT/EP2014/063422

§ 371 Date: Dec. 21, 2015

(30) Foreign Application Priority Data
Jun. 27, 2013 (EP) 13174123.3

(51) Int. Cl. C07D 265/10; C07D 263/22; A01N 43/76; A01N 43/86

(52) U.S. Cl. CPC C07D 265/10; A01N 43/86; C07D 263/22; A01N 43/76

(57) ABSTRACT

The present invention relates to novel strobilurine type compounds, to compositions comprising at least one such compound, to methods for combating phytopathogenic fungi, to the use of such compounds and to seeds coated with at least one such compound.

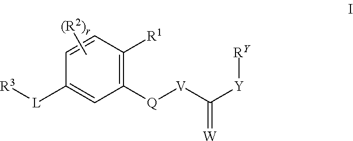

The present invention relates to novel strobilurine type compounds, to compositions comprising at least one such compound, to methods for combating phytopathogenic fungi, to the use of such compounds and to seeds coated with at least one such compound.

WO 01/10825 A1 describes carbamate derivatives and agricultural/horticultural bactericides with fungicidal activity. WO 2008/124092 A2 describes closely related carbamates as fungicides. WO 2010/018676 A1 relates to oxime ether derivatives and bactericides for agricultural and horticultural use.

The compounds according to the present invention differ from those described in the abovementioned publications in that they are characterized by the specific group -Q-V(R^{V})—C(═W)—YR^{Y}.

Compound 3-[[5-[4-(dichloromethyl)thiazol-2-yl]-2-fluoro-phenyl]methyl]oxazolidin-2-one is commercially available and has been disclosed in Aurora Fine Chemicals LLC Catalogue (order number A00.379.922; published online on Jan. 1, 2013).

In many cases, in particular at low application rates, the fungicidal activity of known fungicidal compounds is unsatisfactory. Based on this, it was an object of the present invention to provide compounds having improved activity and/or a broader activity spectrum against phytopathogenic fungi. This objective is achieved by the use of strobilurin type compounds of formula I having good fungicidal activity against phytopathogenic fungi.

Accordingly, the present invention relates to compounds of the formula I

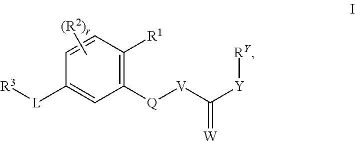

wherein:
R¹ is halogen, cyano, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₁-C₆-alkylthio, C₁-C₆-alkylsulfinyl, C₁-C₆-alkylsulfonyl, C₃-C₆-cycloalkyl or C₃-C₆-cycloalkyl-C₁-C₄-alkyl; wherein the aliphatic and alicyclic moieties of R¹ are unsubstituted or substituted by 1, 2, 3 or up to the maximum number of identical or different groups R^{1a}; wherein
   R^{1a} is halogen, hydroxy, cyano, nitro, C₁-C₄-alkyl, C₁-C₄-alkoxy, C₃-C₆-cycloalkyl, C₁-C₄-haloalkyl or C₁-C₄-haloalkoxy;
R² is halogen, hydroxy, cyano, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl or C₃-C₆-cycloalkyl-C₁-C₄-alkyl; wherein the aliphatic and alicyclic moieties of R² are unsubstituted or substituted by 1, 2, 3 or up to the maximum number of identical or different groups R^{2a}; wherein
   R^{2a} is halogen, hydroxy, cyano, nitro, C₁-C₄-alkyl, C₁-C₄-alkoxy, C₃-C₆-cycloalkyl, C₁-C₄-haloalkyl or C₁-C₄-haloalkoxy;
r is 0, 1, 2 or 3;
L is a direct bond or a divalent group selected from —OCH₂—, —CH₂—, —CH₂CH₂—, —O—, —CH₂—O—N═C(Z)—, —O—N═C(Z)—, —C(Z)═N—O—CH₂—, —CHZ—C(Z)═N—O—CH₂—, —O—N═C(Z)—C(Z)═N—O—CH₂—, —C(═O)—C(Z)═N—O—CH₂— and —C(═N—O—Z)—C(Z)═N—O—CH₂—; wherein the bond depicted on the left side of the divalent group L is attached to R³, and the bond depicted on the right side is attached to the phenyl ring; wherein
   Z is independently selected from hydrogen, amino, C₁-C₄-alkyl, C₁-C₄-haloalkyl and C₁-C₆-alkoxyimino-C₁-C₄-alkyl;
R³ is phenyl or a 5- or 6-membered aromatic heterocycle, wherein the ring member atoms of the heterocycle include besides carbon atoms 1, 2, 3 or 4 heteroatoms independently selected from N, O and S as ring member atoms; wherein the cyclic groups R³ are unsubstituted or substituted by 1, 2, 3, 4 or up to the maximum possible number of identical or different groups R^{3a}; wherein
   R^{3a} is amino, halogen, hydroxy, nitro, cyano, carboxyl, C₁-C₆-alkyl, C₂-C₆-alkenyl, C₂-C₆-alkynyl, C₁-C₆-alkoxy, C₃-C₆-cycloalkyl, C₃-C₆-cycloalkenyl, C₂-C₆-alkenyloxy, C₃-C₆-alkynyloxy, C₁-C₆ alkoxyimino C₁-C₄-alkyl, C₂-C₆-alkenyloxyimino-C₁-C₄-alkyl, C₃-C₆-alkynyloxyimino-C₁-C₄-alkyl, C₁-C₆-alkylamino, C(═O)—(C₁-C₆-alkyl), C(═O)—(C₁-C₆-alkoxy), phenyl, naphthyl or a 3- to 10-membered saturated, partially unsaturated or aromatic mono- or bicyclic heterocycle, wherein the ring member atoms of the heterocycle include besides carbon atoms 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the aforementioned phenyl and heterocycle groups R^{3a} are attached to R³ via a direct bond, an oxygen or sulfur atom, the latter two atoms forming a linker between said residues; and wherein the aliphatic or cyclic groups R^{3a} are unsubstituted or substituted by 1, 2 or 3 or up to the maximum possible number of identical or different groups R^{3b}; wherein
      R^{3b} is halogen, hydroxy, nitro, cyano, carboxyl, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₁-C₆-alkoxyimino-C₁-C₄-alkyl, C₂-C₆-alkenyloxyimino-C₁-C₄-alkyl, C₃-C₆-alkynyloxyimino-C₁-C₄-alkyl, C₁-C₆-alkylthio, C₁-C₆-alkylsulfinyl, C₁-C₆-alkylsulfonyl, C₃-C₆-cycloalkyl, C₃-C₆-cycloalkenyl, phenyl or a 5- or 6-membered saturated, partially unsaturated or aromatic heterocycle; wherein the ring member atoms of the heterocycle include besides carbon atoms 1, 2 or 3 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the aforementioned cyclic groups R^{3b} are attached to R^{3a} via a direct bond, an oxygen or sulfur atom, the latter two atoms forming a linker between said residues; and wherein the aliphatic or cyclic groups R^{3b} are unsubstituted or substituted by 1, 2 or 3 or up to the maximum possible number of identical or different groups selected from halogen, C₁-C₆-alkyl and C₁-C₆-haloalkyl;
Q is a divalent group selected from —O—, —S—, —(NQ^{a})-, —(CQ^{b}Q^{c})-, —C(═N—O-Q^{a})-, —C(═O)— and —C(═S)—; wherein
   Q^{a} is hydrogen, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl, phenyl-C₁-C₄-alkyl, heteroaryl-C₁-C₄-alkyl or C₃-C₆-cycloalkyl-C₁-C₄-alkyl; wherein the aliphatic, alicylic and aromatic moieties of Q^{a} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy;
   Q^{b}, Q^{c} are independently selected from hydrogen, halogen, cyano, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl and C₃-C₆-cycloalkyl-C₁-C₄-alkyl; wherein the aliphatic and alicyclic moieties of Q^{b} and/or Q^{c} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy; or
   Q^{b} and Q^{c} together with the carbon atom to which they are bound form a saturated or partially unsaturated 3-, 4-, 5-, 6- or 7-membered carbocycle or a saturated or partially unsaturated 3-, 4-, 5-, 6- or 7-membered heterocycle, wherein the heterocycle includes beside carbon atoms 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the carbo- and heterocycle are unsubstituted or substituted by 1, 2, 3 or 4 identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy;
V is a divalent group —NR^{V}—;
W is O or S;
Y is a divalent group selected from —O—, —S— and —NY^{a}—; wherein
   Y^{a} is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, phenyl-C₁-C₄-alkyl, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy;
      and wherein
R^{V} and R^{Y}, together with the group —(N—C(═W)—Y)—, form a 5-, 6- or 7-membered saturated heterocycle; wherein the heterocycle includes beside carbon atoms 1, 2, 3 or 4 heteroatoms independently selected from N, O and S as ring member atoms; and wherein 1 or 2 carbon ring member atoms of the heterocycle may be replaced by 1 or 2 groups independently selected from C(═O) and C(═S); and wherein the heterocycle is unsubstituted or substituted by 1, 2, 3 or 4 identical or different groups R^{X}; wherein
   R^{X} is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, C₁-C₆-alkylamino, C₁-C₆-dialkylamino or C₃-C₆-cycloalkylamino; wherein the aliphatic and alicyclic moieties of R^{X} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy;
      and the N-oxides and the agriculturally acceptable salts thereof, with the proviso that 3-[[5-[4-(dichloromethyl)thiazol-2-yl]-2-fluoro-phenyl]methyl]oxazolidin-2-one be excluded.

The present invention also relates to methods for combating phytopathogenic fungi, which process comprises treating the fungi or the materials, plants, the soil or seeds to be protected against fungal attack, with an effective amount of at least one compound of formula I or of an N-oxide or an agriculturally acceptable salt thereof.

The present invention also provides a use of compounds of the formula I and/or their agriculturally useful salts for controlling phytopathogenic fungi. The invention further provides compositions comprising these compounds I and/or their agriculturally acceptable salts. The present invention also relates to seeds treated with at least one such compound or seeds comprising at least one such compound.

Qo inhibitor fungicides, often referred to as strobilurin-type fungicides (Sauter 2007: Chapter 13.2. Strobilurins and other complex III inhibitors. In: Krämer, W.; Schirmer, U. (Ed.) Modern Crop Protection Compounds. Volume 2. Wiley-VCH Verlag 457-495), are conventionally used to control a number of fungal pathogens in crops. Qo inhibitors typically work by inhibiting respiration by binding to a ubihydroquinone oxidation center of a cytochrome bc₁ complex (electron transport complex III) in mitochondria. Said oxidation center is located on the outer side of the inner mitochrondrial membrane. A prime example of the use of Qo inhibitors includes the use of, for example, strobilurins on wheat for the control of Septoria tritici (also known as Mycosphaerella graminicola), which is the cause of wheat leaf blotch. Unfortunately, widespread use of such Qo inhibitors has resulted in the selection of mutant pathogens which are resistant to such Qo inhibitors (Gisi et al., Pest Manag Sci 56, 833-841, (2000). Resistance to Qo inhibitors has been detected in several phytopathogenic fungi such as Blumeria graminis, Mycosphaerella fijiensis, Pseudoperonspora cubensis or Venturia inaequalis. Although several resistance mechanisms have been detected meanwhile (e.g. Jabs et al. Phytomedizin 31, 15-16 (2001); Olaya et al., Pestic Sci 54, 230-236 (1998), the major part of resistance to Qo inhibitors in agricultural uses has been attributed to pathogens containing a single amino acid residue substitution G143A in the cytochrome b gene for their cytochrome bc₁ complex, the target protein of Qo inhibitors. See, for example, Lucas, Pestic Outlook 14(6), 268-70 (2003); and Fraaije et al., Phytopathol 95(8), 933-41 (2005), (which both are expressly incorporated by reference herein). Thus, new methods and compositions are desirable for controlling pathogen induced diseases in crops comprising plants subjected to pathogens that are resistant to Qo inhibitors. Furthermore, in many cases, in particular at low application rates, the fungicidal activity of the known fungicidal strobilurin analogue compounds is unsatisfactory, especially in case that a high proportion of the fungal pathogens contain a mutation in the mitochondrial cytochrome b gene conferring resistance to Qo inhibitors. Based on this, it was also an object of the present invention to provide compounds having improved activity and/or a broader activity spectrum against phytopathogenic harmful fungi.

“Qo inhibitor,” as used herein, includes any substance that is capable of diminishing and/or inhibiting respiration by binding to a ubihydroquinone oxidation center of a cytochrome bc₁ complex in mitochondria. The oxidation center is typically located on the outer side of the inner mitochrondrial membrane.

Accordingly, the invention also relates to the use of such strobilurine type compounds of formula I and the N-oxides and the salts thereof for combating phytopathogenic fungi containing a mutation in the mitochondrial cytochrome b gene conferring resistance to Qo inhibitors, and to methods for combating such fungi.

Agriculturally useful salts of the compounds I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

Compounds I can exist as one or more stereoisomers. The various stereoisomers include enantiomers, diastereomers, atropisomers arising from restricted rotation about a single bond of asymmetric groups and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds I can be present in different crystal modifications whose biological activity may differ. They also form part of the subject matter of the present invention. The compounds of formula I can be present in atropisomers arising from restricted rotation about a single bond of asymmetric groups. They also form part of the subject matter of the present invention.

In respect of the variables, the embodiments of the intermediates obtained during preparation of compounds I correspond to the embodiments of the compounds of formula I. The term “compounds I” refers to compounds of formula I.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, for example methyl, ethyl, propyl, pentyl, hexyl, 1-methylethyl, butyl, 1-methylpropyl, 2-methylpropyl, and 1,1-dimethylethyl. The term “C₁-C₄-alkyl” refers to a straight-chained or branched saturated hydrocarbon group having 1 to 4 carbon atoms, for example methyl, ethyl, propyl, butyl, 1-methylethyl, 1-methylpropyl, 2-methylpropyl, and 1,1-dimethylethyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. The term “C₁-C₄-haloalkyl” refers to a straight-chained or branched alkyl group having 1 to 4 carbon atoms (as defined above), wherein some or all of the hydrogen atoms in these groups may be replaced by halogen atoms as mentioned above. Representative examples of C₁-C₄-haloalkyl are given above for the C₁-C₆-haloalkyl compounds.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy. The term “C₁-C₄-alkoxy” refers to a straight-chain or branched alkyl group having 1 to 4 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “C₂-C₆-alkenyloxy” refers to a straight-chain or branched alkenyl group having 2 to 6 carbon atoms (as defined above) which is bonded via an oxygen, at any position in the alkenyl group.

The term “C₂-C₆-alkynyloxy” refers to a straight-chain or branched alkynyl group having 2 to 6 carbon atoms (as defined above) which is bonded via an oxygen, at any position in the alkynyl group. The term “C₃-C₆-alkynyloxy” refers to a straight-chain or branched alkynyl group having 3 to 6 carbon atoms (as defined above) which is bonded via an oxygen, at any position in the alkynyl group.

The term “C₁-C₆-haloalkoxy” refers to a C₁-C₆-alkoxy group as defined above, wherein some or all of the hydrogen atoms may be replaced by halogen atoms as mentioned above, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. The term “C₁-C₄-haloalkoxy” refers to a C₁-C₄-alkoxy group as defined above, wherein some or all of the hydrogen atoms may be replaced by halogen atoms as mentioned above. Representative examples of C₁-C₄-haloalkoxy are given above for the C₁-C₆-haloalkoxy compounds.

The terms “phenyl-C₁-C₄-alkyl” or “heteroaryl-C₁-C₄-alkyl” refer to alkyl having 1 to 4 carbon atoms (as defined above), wherein one hydrogen atom of the alkyl radical is replaced by a phenyl or an aromatic, heterocyclic radical respectively.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₆-alkoxy-C₁-C₄-alkyl” refers to alkyl having 1 to 6 carbon atoms (as defined above), wherein one hydrogen atom of the alkyl radical is replaced by a C₁-C₆-alkoxy group (as defined above).

The term “C₁-C₆-alkylthio” as used herein refers to straight-chain or branched alkyl groups having 1 to 6 carbon atoms (as defined above) bonded via a sulfur atom. Accordingly, the term “C₁-C₆-haloalkylthio” as used herein refers to straight-chain or branched haloalkyl group having 1 to 6 carbon atoms (as defined above) bonded through a sulfur atom, at any position in the haloalkyl group.

The term “C₁-C₆-alkylsulfinyl” refers to straight-chain or branched alkyl groups having 1 to 6 carbon atoms (as defined above) bonded through a —S(═O)— moiety, at any position in the alkyl group, for example methylsulfinyl and ethylsulfinyl, and the like. Accordingly, the term “C₁-C₆-haloalkylsulfinyl” refers to straight-chain or branched haloalkyl group having 1 to 6 carbon atoms (as defined above), bonded through a —S(═O)— moiety, at any position in the haloalkyl group.

The term “C₁-C₆-alkylsulfonyl” refers to straight-chain or branched alkyl groups having 1 to 6 carbon atoms (as defined above), bonded through a —S(═O)₂— moiety, at any position in the alkyl group, for example methylsulfonyl. Accordingly, the term “C₁-C₆-haloalkylsulfonyl” refers to straight-chain or branched haloalkyl group having 1 to 6 carbon atoms (as defined above), bonded through a —S(═O)₂— moiety, at any position in the haloalkyl group.

The term “C₁-C₆-alkylamino” as used herein refers to a straight-chain or branched alkyl group having 1 to 6 carbon atoms (as defined above) bonded via an NH-group.

The term “C₁-C₆-dialkylamino” as used herein refers to two identical or different straight-chain or branched alkyl groups having 1 to 6 carbon atoms (as defined above) bonded via a nitrogen atom.

The term “C₃-C₆-cycloalkylamino” as used herein refers to a cycloalkyl group having 3 to 6 carbon atoms (as defined below) bonded via an NH-group.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl. The term “C₃-C₆-alkynyl” refers to a straight-chain or branched unsaturated hydrocarbon radical having 3 to 6 carbon atoms and containing at least one triple bond, such as 1-propynyl, 2-propynyl (propargyl), 1-butynyl, 2-butynyl, 3-butynyl, 1-methyl-2-propynyl.

The term “C₃-C₈-cycloalkyl” refers to monocyclic saturated hydrocarbon radicals having 3 to 8 carbon ring members such as cyclopropyl (C₃H₅), cyclobutyl, cyclopentyl, cyclohexyl, cycloheptyl or cyclooctyl. The term “C₃-C₆-cycloalkyl” refers to monocyclic saturated hydrocarbon radicals having 3 to 6 carbon ring members such as cyclopropyl (C₃H₅), cyclobutyl, cyclopentyl, or cyclohexyl.

The term “C₃-C₈-cycloalkyl-C₁-C₄-alkyl” refers to a cycloalkyl radical having 3 to 8 carbon atoms (as defined above), which is bonded via a C₁-C₄-alkyl group as defined above. The term “C₃-C₆-cycloalkyl-C₁-C₄-alkyl” refers to a cycloalkyl radical having 3 to 6 carbon atoms (as defined above), which is bonded via a C₁-C₄-alkyl group as defined above.

The term “C₃-C₈-cycloalkyloxy” refers to a cycloalkyl radical having 3 to 8 carbon atoms (as defined above), which is bonded via an oxygen.

The term “C(═O)—(C₁-C₄-alkyl)” refers to a radical which is attached through the carbon atom of the C(═O) group as indicated by the number valence of the carbon atom.

The term “C₁-C₆-alkoxyimino-C₁-C₄-alkyl” refers to a radical which is attached through a carbon atom of the C₁-C₄-alkyl chain, wherein one —CH₂— group is replaced by a —C(═N—O—(C₁-C₆-alkoxy))-group. Likewise the terms C₂-C₆-alkenyloxyimino-C₁-C₄-alkyl and C₃-C₆-alkynyloxyimino-C₁-C₄-alkyl are to be construed.

The term “saturated or partially unsaturated 3-, 4-5-, 6- or 7-membered carbocycle” is to be understood as meaning both saturated or partially unsaturated carbocycles having 3, 4, 5, 6 or 7 ring members. Examples include cyclopropyl, cyclobutyl, cyclobutenyl, cyclopentyl, cyclopentenyl, cyclopentadienyl, cyclohexyl, cyclohexenyl, cyclohexadienyl, cycloheptyl, cycloheptenyl, cycloheptadienyl, and the like.

The term “saturated or partially unsaturated 3-, 4-, 5-, 6-, or 7-membered heterocycle, wherein the ring member atoms of the heterocycle include beside carbon atoms 1, 2, 3 or 4 heteroatoms independently selected from the group of N, O and S”, is to be understood as meaning both saturated and partially unsaturated heterocycles, for example:
a 3- or 4-membered saturated heterocycle which contains 1 or 2 heteroatoms independently selected from the group consisting of N, O and S as ring members such as oxirane, aziridine, thiirane, oxetane, azetidine, thiethane, [1,2]dioxetane, [1,2]dithietane, [1,2]diazetidine; and
a 5- or 6-membered saturated or partially unsaturated heterocycle which contains 1, 2 or 3 heteroatoms independently selected from the group consisting of N, O and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and
a 7-membered saturated or partially unsaturated heterocycle such as tetra- and hexahydroazepinyl, such as 2,3,4,5-tetrahydro[1H]azepin-1-,-2-,-3-,-4-,-5-,-6- or -7-yl, 3,4,5,6-tetrahydro[2H]azepin-2-,-3-,-4-,-5-,-6- or -7-yl, 2,3,4,7-tetrahydro[1H]azepin-1-,-2-,-3-,-4-,-5-,-6- or -7-yl, 2,3,6,7-tetrahydro[1H]azepin-1-,-2-,-3-,-4-,-5-,-6- or -7-yl, hexahydroazepin-1-,-2-,-3- or -4-yl, tetra- and hexahydrooxepinyl such as 2,3,4,5-tetrahydro[1H]oxepin-2-,-3-,-4-,-5-,-6- or -7-yl, 2,3,4,7-tetrahydro[1H]oxepin-2-,-3-,-4-,-5-,-6- or -7-yl, 2,3,6,7-tetrahydro[1H]oxepin-2-,-3-,-4-,-5-,-6- or -7-yl, hexahydroazepin-1-,-2-,-3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals; and

The term “5- or 6-membered heteroaryl” or the term “5- or 6 membered aromatic heterocycle” (also referred to as aromatic, heterocyclic radical) refers to aromatic ring systems including beside carbon atoms, 1, 2, 3 or 4 heteroatoms independently selected from the group consisting of N, O and S, for example,
a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or
a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

The term “3- to 10-membered saturated, partially unsaturated or aromatic mono- or bicyclic heterocycle”, refers to a saturated, partially unsaturated or aromatic” monocyclic or bicyclic ring system, wherein the ring member atoms of the heterocycle include besides carbon atoms contain 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S). The “3- to 10-membered saturated, partially unsaturated or aromatic mono- or bicyclic heterocycle” also includes monocyclic 5- or 6-membered saturated, partially unsaturated or aromatic systems, which are fused to a benzo ring system such as in benzodioxole, benzodiazole, benzothiazole, indole, indazole, benzimidazole, benzoxazole, and the like.

In respect of the the variables, the embodiments of the intermediates correspond to the embodiments of the compounds I.

Preference is given to those compounds I and where applicable also to compounds of all sub-formulae provided herein, e. g. formulae I.A, I.B and I.C, and to the intermediates such as compounds II and III, wherein the substituents and variables (such as R¹, R^{1a}, R², R^{2a}, R³, R^{3a}, R^{3b}, L, r, Y, R^{Y}, W, Q, Q^{a}, Q^{b}, Q^{c}, R^{V}) have independently of each other or more preferably in combination (any possible combination of 2 or more substituents as defined herein) the following meanings:

R¹ according to the invention is halogen, cyano, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₁-C₆-alkylthio, C₁-C₆-alkylsulfinyl, C₁-C₆-alkylsulfonyl, C₃-C₆-cycloalkyl or C₃-C₆-cycloalkyl-C₁-C₄-alkyl; wherein the aliphatic and alicyclic moieties of R¹ are unsubstituted or substituted by 1, 2, 3 or up to the maximum number of identical or different groups R^{1a} as defined or preferably defined below; in particular R^{1a} is F or Cl.

In a preferred embodiment of the invention R¹ is halogen, cyano, C₁-C₆-alkyl, C₁-C₆-alkoxy, C₂-C₆-alkenyl, C₂-C₆-alkynyl; wherein the aliphatic moieties of R¹ are unsubstituted or substituted by 1, 2, 3 or up to the maximum number of identical or different groups R^{1a} as defined or preferably defined below; in particular R^{1a} is F or Cl. In another preferred embodiment R¹ is halogen, cyano, C₁-C₆-alkyl, C₁-C₆-alkoxy, C₁-C₆-haloalkyl, C₁-C₆-haloalkoxy, C₂-C₆-alkenyl or C₂-C₆-alkynyl. In another preferred embodiment R¹ is CH₃, CH₂CH₃, OCH₃, OCH₂CH₃, CF₃, CHF₂, OCF₃, OCHF₂, SCF₃, cyano, Cl, F or Br. In a further embodiment R¹ is F, Cl, cyano, CH₃ or OCH₃; in particular F or Cl.

R^{1a} according to the invention is halogen, hydroxy, cyano, nitro, C₁-C₄-alkyl, C₁-C₄-alkoxy, C₃-C₆-cycloalkyl, C₁-C₄-haloalkyl or C₁-C₄-haloalkoxy. In a preferred embodiment of the invention R^{1a} is halogen, C₁-C₄-alkyl or C₁-C₄-alkoxy; more preferably R^{1a} is halogen, in particular F or Cl.

R² according to the invention is halogen, hydroxy, cyano, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl or C₃-C₆-cycloalkyl-C₁-C₄-alkyl; wherein the aliphatic and alicyclic moieties of R² are unsubstituted or substituted by 1, 2, 3 or up to the maximum number of identical or different groups R^{2a} as defined or preferably defined below; in particular R^{2a} is F or Cl. In a preferred embodiment of the invention R² is halogen, cyano, C₁-C₆-alkyl or C₁-C₆-alkoxy; wherein the aliphatic moieties of R² are unsubstituted or substituted by 1, 2, 3 or up to the maximum number of identical or different groups R^{2a} as defined or preferably defined below; in particular R^{2a} is F or Cl.

In another preferred embodiment R² is CH₃, CH₂CH₃, OCH₃, OCH₂CH₃, CF₃, CHF₂, OCF₃, OCHF₂, cyano, Cl, F or Br. In a further embodiment R¹ is F, Cl, cyano, CH₃ or OCH₃; in particular F or Cl.

R^{2a} according to the invention is halogen, hydroxy, cyano, nitro, C₁-C₄-alkyl, C₁-C₄alkoxy, C₃-C₆-cycloalkyl, C₁-C₄-haloalkyl or C₁-C₄-haloalkoxy. In a preferred embodiment of the invention R^{2a} is halogen, C₁-C₄-alkyl or C₁-C₄alkoxy; more preferably R^{2a} is halogen, in particular Cl or F.

According to the invention r is 0, 1, 2 or 3. In one embodiment of the invention r is 0, 1 or 2. In another embodiment of the invention r is 0 or 1. In yet another embodiment of the invention r is 1 or 2. In a preferred embodiment of the invention r is 0. In a further preferred embodiment of the invention r is 1. In still another preferred embodiment of the invention r is 2.

L according to the invention is a direct bond or a divalent group selected from —OCH₂—, —CH₂—, —CH₂CH₂—, —CH₂—O—N═C(Z)—, —O—N═C(Z)—, —C(Z)═N—O—CH₂—, —CHZ—C(Z)═N—O—CH₂—, —O—N═C(Z)—C(Z)═N—O—CH₂—, —C(═O)—C(Z)═N—O—CH₂—, —C(═N—O—Z)—C(Z)═N—O—CH₂— and (Z)C(═N—O—Z)—C(═N—O—Z)—C(Z)═N—O—CH₂—; wherein the bond depicted on the left side of the divalent group L is attached to R³ and the bond depicted on the right side is attached to the phenyl ring; wherein Z is as defined or preferably defined below; in particular Z is independently selected from hydrogen and CH₃.

In one embodiment of the invention L is a divalent group selected from —OCH₂—, —CH₂— and —CH₂CH₂—, wherein the bond depicted on the left side of the group —OCH₂— is attached to R³ and the bond depicted on the right side is attached to the phenyl ring. In a preferred embodiment of the invention L is —OCH₂— or —CH₂—, in particular —OCH₂—.

In another aspect of the invention L is —CH₂—O—N═C(Z)— or —O—N═C(Z)—; wherein Z is as defined or preferably defined below; in particular Z is independently selected from hydrogen and CH₃. In one preferred embodiment L is —CH₂—O—N═C(Z)—; wherein Z is as defined or preferably defined below. In another preferred embodiment L is —O—N═C(Z)—; wherein Z is as defined or preferably defined below; in particular Z is independently selected from hydrogen and CH₃.

In a further aspect of the invention L is —C(Z)═N—O—CH₂— or —CHZ—C(Z)═N—O—CH₂—; wherein Z is as defined or preferably defined below; in particular Z is independently selected from hydrogen and CH₃.

In still another aspect of the invention L is —O—N═C(Z)—C(Z)═N—O—CH₂—, —C(═O)—C(Z)═N—O—CH₂— or —C(═N—O—Z)—C(Z)═N—O—CH₂—; wherein Z is as defined or preferably defined below; in particular Z is independently selected from hydrogen and CH₃.

Z according to the invention is independently selected from hydrogen, amino, C₁-C₄-alkyl, C₁-C₄-haloalkyl and C₁-C₆-alkoxyimino-C₁-C₄-alkyl. In a preferred aspect Z is independently selected from hydrogen, C₁-C₄-alkyl and C₁-C₆-alkoxyimino-C₁-C₄-aralkyl, in particular from hydrogen and CH₃.

R³ according to the invention is phenyl or a 5- or 6-membered aromatic heterocycle, wherein the ring member atoms of the heterocycle include beside carbon atoms 1, 2, 3 or 4 heteroatoms independently selected from N, O and S as ring member atoms; wherein the cyclic groups R³ are unsubstituted or substituted by 1, 2, 3 or 4 identical or different groups R^{3a} as defined or preferably defined below; in particular R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br.

According to a further embodiment R³ is substituted by 1, 2 or 3 identical or different groups R^{3a} as defined or preferably defined below; in particular R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br.

In one embodiment of the invention R³ is phenyl; wherein the phenyl ring is unsubstituted or substituted by 1, 2, 3 or 4 identical or different groups R^{3a} as defined or preferably defined below; in particular R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br.

In another embodiment of the invention R³ is phenyl; wherein the phenyl ring is unsubstituted or substituted by 1 or 2 identical or different groups R^{3a} as defined or preferably defined below; in particular R^{3a} is a 5-membered aromatic heterocycle, methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br; and wherein at least one of said groups R^{3a} is a 5-membered aromatic heterocycle, wherein the ring member atoms of the heterocycle include beside carbon atoms 1, 2 or 3 heteroatoms independently selected from the group of N, O and S as ring member atoms; and wherein said aromatic heterocycle is unsubstituted or substituted by 1, 2 or 3 identical or different groups R^{3b} as defined or preferably defined below; in particular R^{3b} is CH₃, OCH₃, cyano, F or Cl. In another embodiment of the invention R³ is a 5- or 6-membered aromatic heterocycle, wherein the ring member atoms of the heterocycle include beside carbon atoms 1, 2, 3 or 4 heteroatoms independently selected from N, O and S as ring member atoms; wherein the aromatic groups R³ are unsubstituted or substituted by 1, 2, 3 or 4 identical or different groups R^{3a} as defined or preferably defined below; in particular R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br.

In a further embodiment R³ is a 5-membered aromatic heterocycle, wherein the ring member atoms of the heterocycle include beside carbon atoms 1, 2 or 3 heteroatoms independently selected from the group of N, O and S as ring member atoms; wherein the aromatic heterocycle is unsubstituted or substituted by 1, 2, 3 or 4 identical or different groups R^{3a} as defined or preferably defined below; in particular R^{3a} is phenyl, methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br; and wherein the phenyl in R^{3a} is unsubstituted or substituted by 1, 2 or 3 identical or different groups R^{3b} as defined or preferably defined below; in particular R^{3b} is CH₃, CH₂CH₃, OCH₃, OCH₂CH₃, CF₃, CHF₂, OCF₃, OCHF₂, SCF₃, SCHF₂, cyano, Cl or F.

Preferably said aromatic heterocycle R³ is pyrazolyl, imidazolyl, oxazolyl, isoxazolyl, thiazolyl, 1,2,4-triazolyl, 1,2,4-oxadiazolyl, 1,3,4-oxadiazolyl or 1,2,4-thiadiazolyl.

According to a further preferred embodiment R³ is pyrazolyl, imidazolyl, oxazolyl, isoxazolyl, thiazolyl, 1,2,4-triazolyl, 1,2,4-oxadiazolyl, 1,3,4-oxadiazolyl or 1,2,4-thiadiazolyl; wherein said aromatic heterocycles are substituted by 1 or 2 identical or different groups R^{3a} as defined or preferably defined below; in particular R^{3a} is phenyl, methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br; and wherein at least one of said groups R^{3a} is phenyl, which is unsubstituted or substituted by 1, 2, 3 or 4 identical or different groups R^{3b} as defined or preferably defined below; in particular R^{3b} is CH₃, CH₂CH₃, OCH₃, OCH₂CH₃, CF₃, CHF₂, OCF₃, OCHF₂, SCF₃, SCHF₂, cyano, Cl or F. In another aspect of the invention R³ is pyrazolyl or 1,2,4-triazolyl; wherein said heterocycles are unsubstituted or substituted by 1 or 2 identical or different groups R^{3a} as defined or preferably defined below; in particular R^{3a} is phenyl, methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br; and wherein at least one of the groups R^{3a} is phenyl, which is unsubstituted or substituted by 1, 2, 3 or 4 identical or different groups R^{3b} as defined or preferably defined below; in particular R^{3b} is CH₃, CH₂CH₃, OCH₃, OCH₂CH₃, CF₃, CHF₂, OCF₃, OCHF₂, SCF₃, SCHF₂, cyano, Cl or F; and wherein said group R^{3a} being phenyl and the group L are attached to the 5-membered heterocycle R³ in a 1,3-substitution pattern, i.e. attached to ring member atoms of the ring R³, which are not directly connected.

In yet another aspect of the invention R³ is 1-phenylpyrazol-3-yl, wherein the phenyl group is unsubstituted or substituted by 1, 2, 3 or 4 identical or different substituents independently selected from CH₃, CH₂CH₃, OCH₃, OCH₂CH₃, CF₃, CHF₂, OCF₃, OCHF₂, SCF₃, SCHF₂, cyano, Cl and F.

In another aspect of the invention R³ is a 6-membered aromatic heterocycle, wherein the ring member atoms of said heterocycle include beside carbon atoms 1, 2 or 3 nitrogen atoms as ring member atoms; wherein said heterocycle is unsubstituted or substituted by 1, 2, 3 or 4 identical or different groups R^{3a} as defined or preferably defined below; in particular R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br; preferably said heteroaryl is pyridinyl or pyrimidinyl.

In a further preferred embodiment R³ is pyridinyl or pyrimidinyl; wherein said heterocycles are unsubstituted or substituted by 1 or 2 identical or different groups R^{3a}; in particular R^{3a} is phenyl, methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br; and wherein at least one of the groups R^{3a} is phenyl, which is unsubstituted or substituted by 1, 2, 3 or 4 identical or different groups R^{3b} as defined or preferably defined below; in particular R^{3b} is CH₃, CH₂CH₃, OCH₃, OCH₂CH₃, CF₃, CHF₂, OCF₃, OCHF₂, SCF₃, SCHF₂, cyano, Cl or F; and wherein said group R^{3a} being phenyl and the group L are attached to the 6-membered heterocycle R³ in a 1,4-substitution pattern, i.e. attached to opposite ring member atoms of the pyridin or pyrimidin ring R³.

In a more preferred embodiment R³ is a pyridinyl ring, which is attached to L in 2-position and which is further unsubstituted or substituted by 1, 2 or 3 identical or different groups R^{3a} as defined or preferably defined below. In another more preferred embodiment R³ is a pyridinyl ring, which is attached to L in 2-position and which is substituted by one group R^{3a} in 6-position and wherein R^{3a} is as defined or preferably defined below; in particular R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, cyano, Cl, F or Br.

Particularly preferred embodiments of the invention relate to compounds I, wherein the group R³ in each case is one of the radicals R3-1 to R3-225 in Table A, wherein # indicates the point of attachment to the linker moiety L:

TABLE A

| Line | R³ |
|---|---|
| R3-1 | 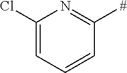 |
| R3-2 | 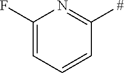 |
| R3-3 | 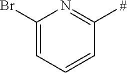 |
| R3-4 | 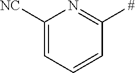 |
| R3-5 | 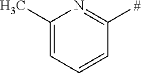 |
| R3-6 | 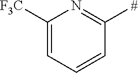 |
| R3-7 | 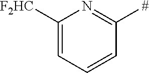 |
| R3-8 | 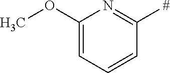 |
| R3-9 | 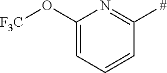 |
| R3-10 | 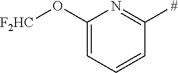 |
| R3-11 | 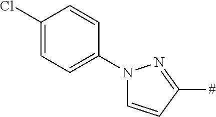 |
| R3-12 | 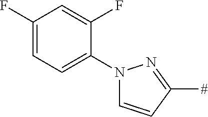 |
| R3-13 | 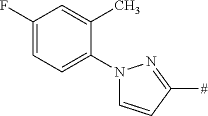 |
| R3-14 | 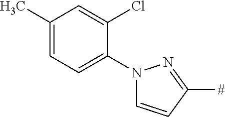 |
| R3-15 | 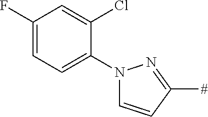 |
| R3-16 | 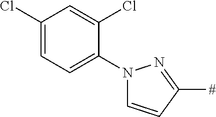 |
| R3-17 | 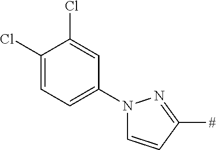 |
| R3-18 | 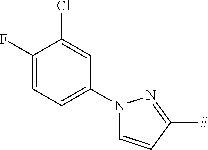 |
| R3-19 | 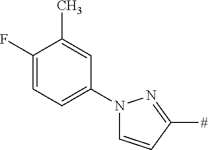 |
| R3-20 | 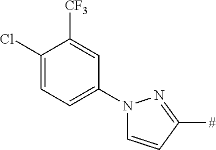 |
| R3-21 | 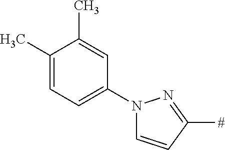 |
| R3-22 | 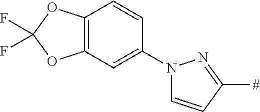 |
| R3-23 | 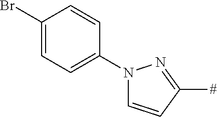 |
| R3-24 | 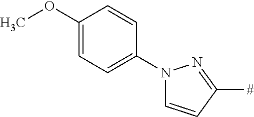 |
| R3-25 | 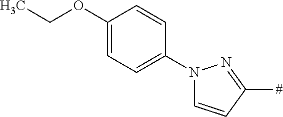 |
| R3-26 | 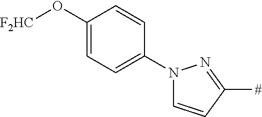 |
| R3-27 | 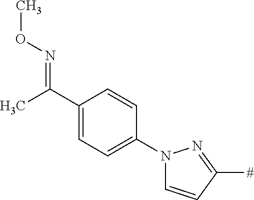 |
| R3-28 | 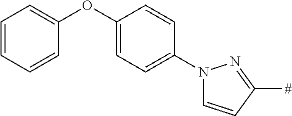 |
| R3-29 | 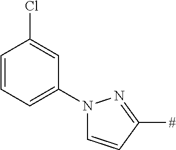 |
| R3-30 | 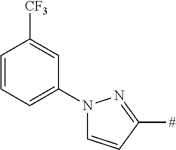 |
| R3-31 | 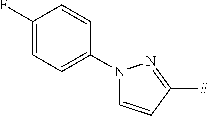 |
| R3-32 | 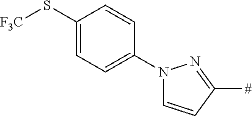 |
| R3-33 | 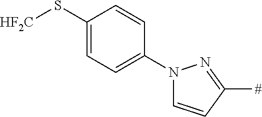 |
| R3-34 | 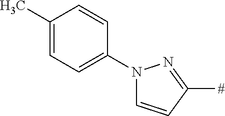 |
| R3-35 | 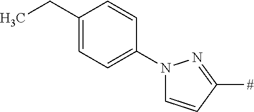 |
| R3-36 | 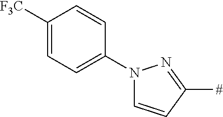 |
| R3-37 | 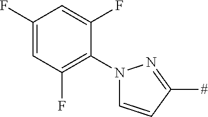 |
| R3-38 | 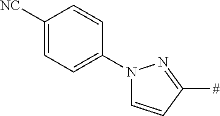 |
| R3-39 | 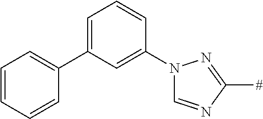 |
| R3-40 | 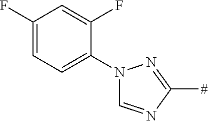 |
| R3-41 | 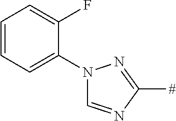 |
| R3-42 | 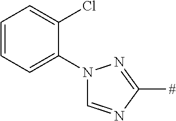 |
| R3-43 | 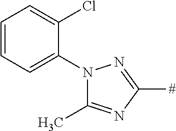 |
| R3-44 | 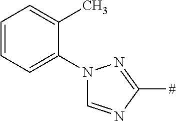 |
| R3-45 | 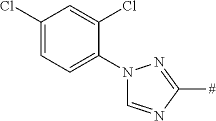 |
| R3-46 | 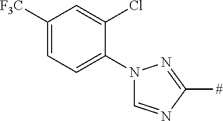 |
| R3-47 | 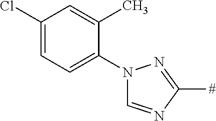 |
| R3-48 | 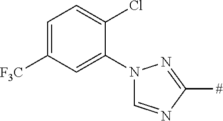 |
| R3-49 | 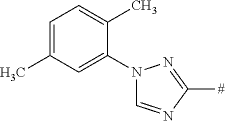 |
| R3-50 | 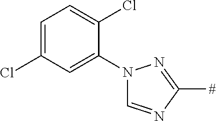 |
| R3-51 | 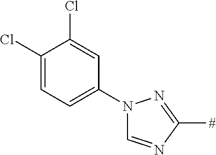 |
| R3-52 | 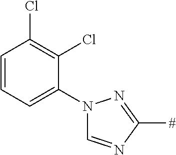 |
| R3-53 | 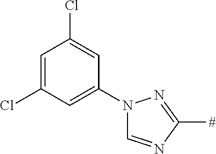 |
| R3-54 | 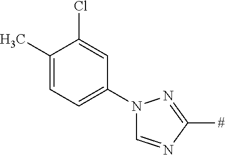 |
| R3-55 | 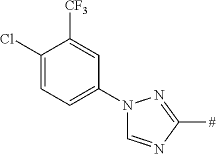 |
| R3-56 | 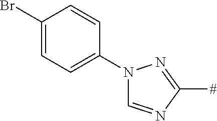 |
| R3-57 | 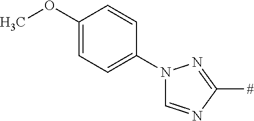 |
| R3-58 | 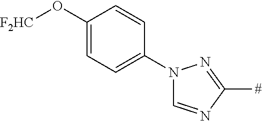 |
| R3-59 | 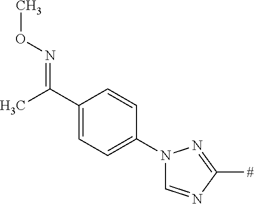 |
| R3-60 | 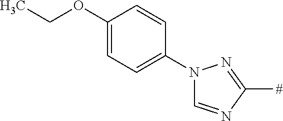 |
| R3-61 | 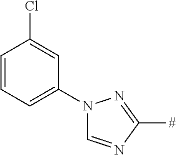 |
| R3-62 | 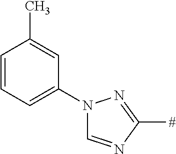 |
| R3-63 | 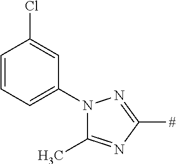 |
| R3-64 | 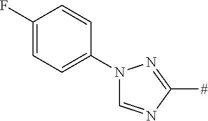 |
| R3-65 | 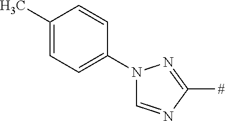 |
| R3-66 | 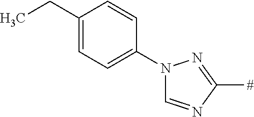 |
| R3-67 | 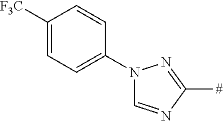 |
| R3-68 | 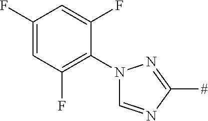 |
| R3-69 | 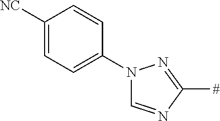 |
| R3-70 | 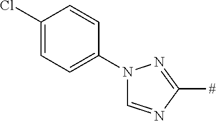 |
| R3-71 | 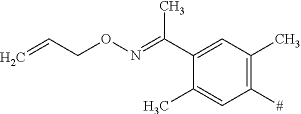 |
| R3-72 | 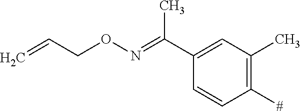 |
| R3-73 | 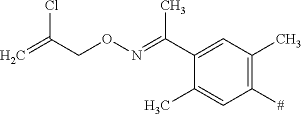 |
| R3-74 | 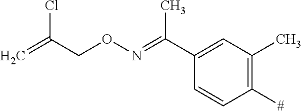 |
| R3-75 | 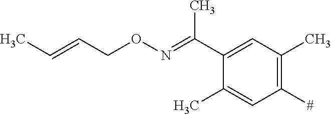 |
| R3-76 | 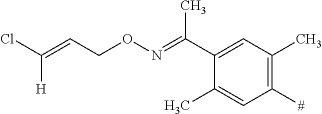 |
| R3-77 | 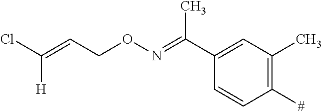 |
| R3-78 | 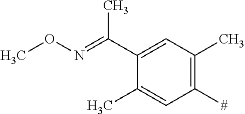 |
| R3-79 | 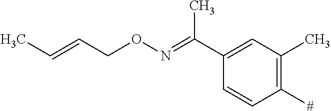 |
| R3-80 | 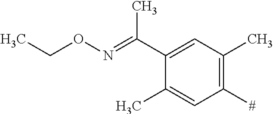 |
| R3-81 | 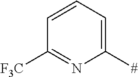 |
| R3-82 | 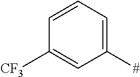 |
| R3-83 | 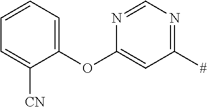 |
| R3-84 | 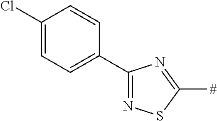 |
| R3-85 | 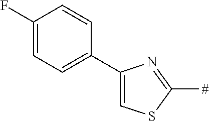 |
| R3-86 | 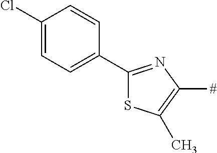 |
| R3-87 | 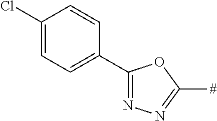 |
| R3-88 | 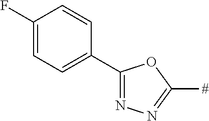 |
| R3-89 | 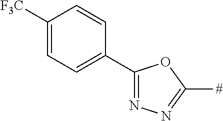 |
| R3-90 | 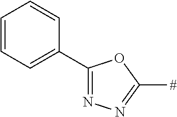 |
| R3-91 | 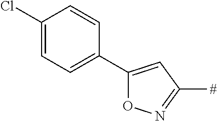 |
| R3-92 | 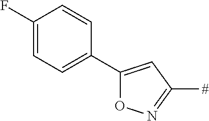 |
| R3-93 | 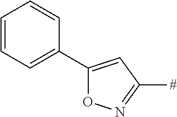 |
| R3-94 | 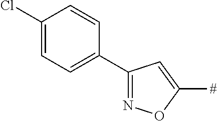 |
| R3-95 | 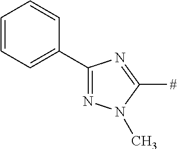 |
| R3-96 | 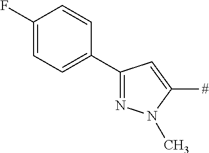 |
| R3-97 | 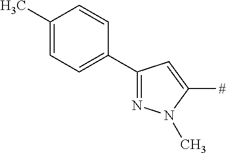 |
| R3-98 | 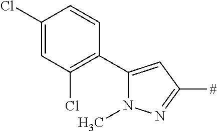 |
| R3-99 | 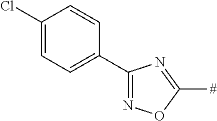 |
| R3-100 | 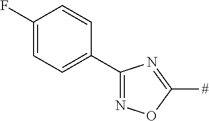 |
| R3-101 | 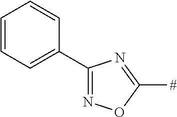 |
| R3-102 | 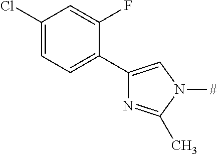 |
| R3-103 | 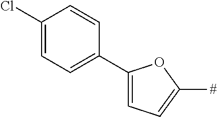 |
| R3-104 | 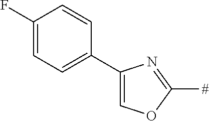 |
| R3-105 | 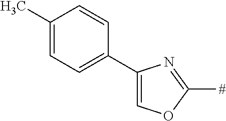 |
| R3-106 | 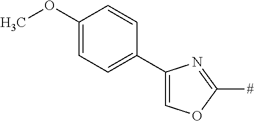 |
| R3-107 | 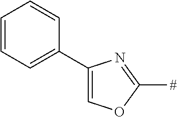 |
| R3-108 | 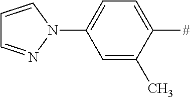 |
| R3-109 | 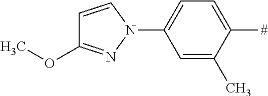 |
| R3-110 | 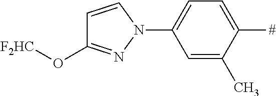 |
| R3-111 | 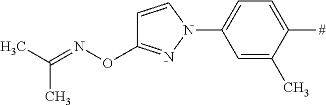 |
| R3-112 | 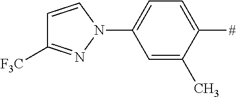 |
| R3-113 | 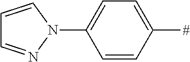 |
| R3-114 | 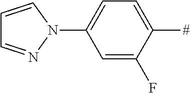 |
| R3-115 | 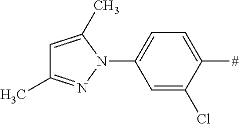 |
| R3-116 | 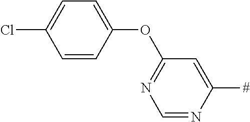 |
| R3-117 | 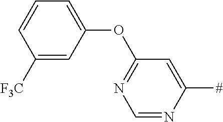 |
| R3-118 | 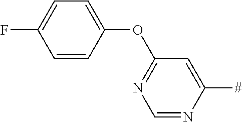 |
| R3-119 | 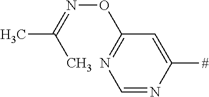 |
| R3-120 | 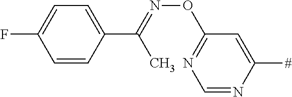 |
| R3-121 | 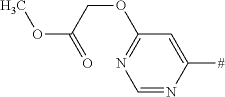 |
| R3-122 | 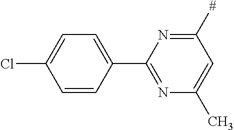 |
| R3-123 | 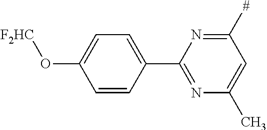 |
| R3-124 | 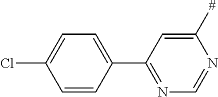 |
| R3-125 | 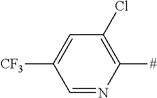 |
| R3-126 | 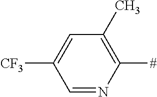 |
| R3-127 | 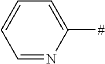 |
| R3-128 | 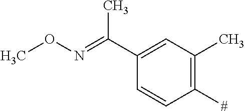 |
| R3-129 | 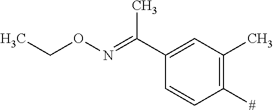 |
| R3-130 | 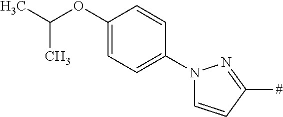 |
| R3-131 | 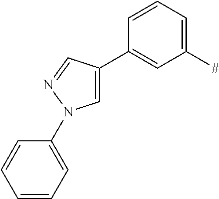 |
| R3-132 | 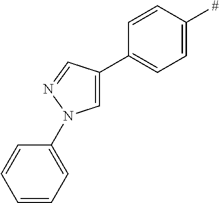 |
| R3-133 | 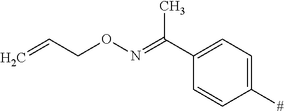 |
| R3-134 | 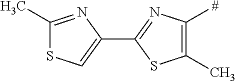 |
| R3-135 | 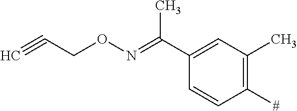 |
| R3-136 | 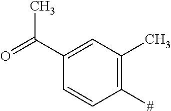 |
| R3-137 | 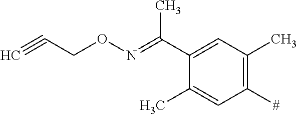 |
| R3-138 | 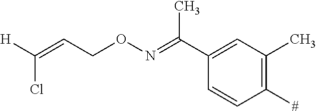 |
| R3-139 | 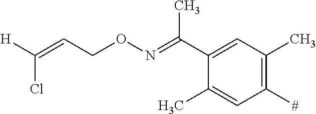 |
| R3-140 | 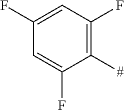 |
| R3-141 | 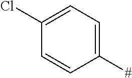 |
| R3-142 | 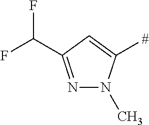 |
| R3-143 | 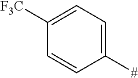 |
| R3-144 | 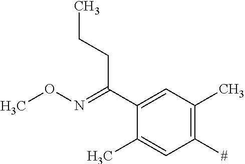 |
| R3-145 | 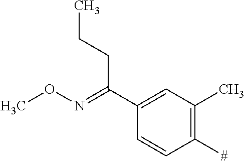 |
| R3-146 | 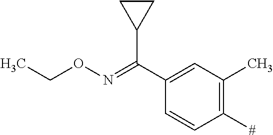 |
| R3-147 | 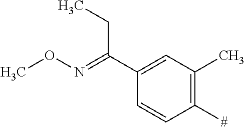 |
| R3-148 | 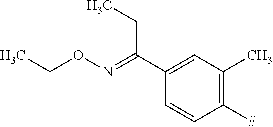 |
| R3-149 | 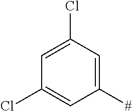 |
| R3-150 | 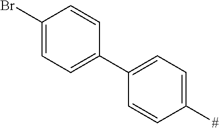 |
| R3-151 | 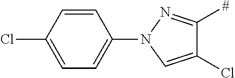 |
| R3-152 | 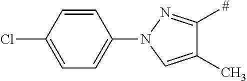 |
| R3-153 | 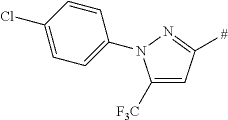 |
| R3-154 | 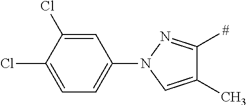 |
| R3-155 | 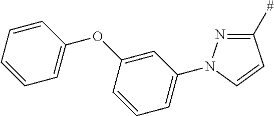 |
| R3-156 | 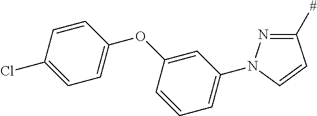 |
| R3-157 | 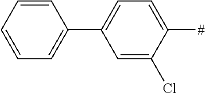 |
| R3-158 | 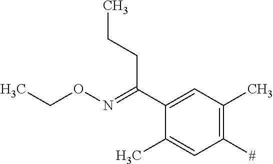 |
| R3-159 | 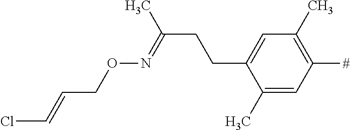 |
| R3-160 | 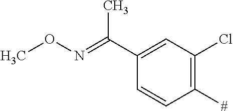 |
| R3-161 | 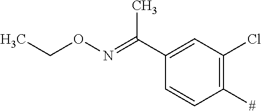 |
| R3-162 | 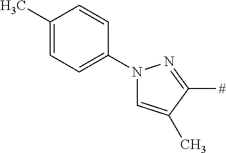 |
| R3-163 | 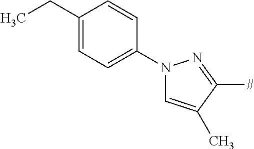 |
| R3-164 | 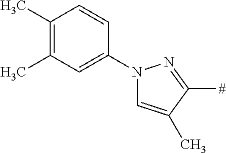 |
| R3-165 | 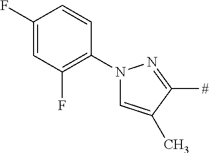 |
| R3-166 | 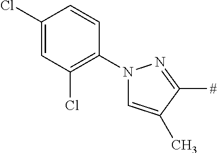 |
| R3-167 | 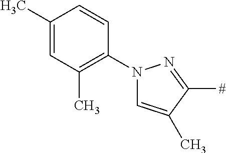 |
| R3-168 | 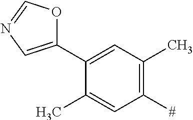 |
| R3-169 | 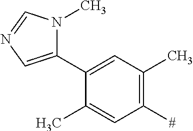 |
| R3-170 | 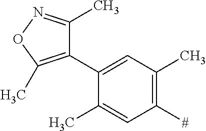 |
| R3-171 | 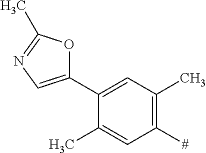 |
| R3-172 | 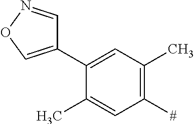 |
| R3-173 | 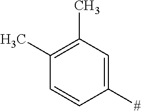 |
| R3-174 | 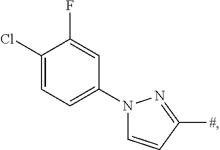 |
| R3-175 | 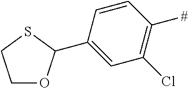 |
| R3-176 | 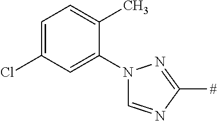 |
| R3-177 | 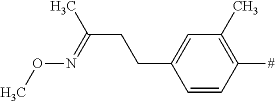 |
| R3-178 | 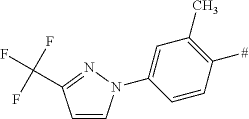 |
| R3-179 | 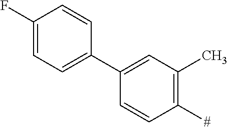 |
| R3-180 | 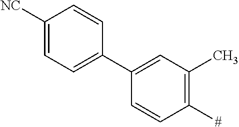 |
| R3-181 | 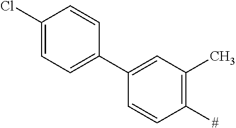 |
| R3-182 | 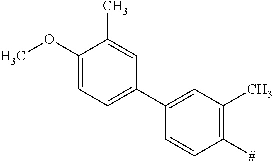 |
| R3-183 | 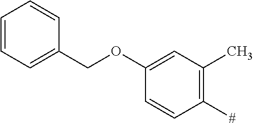 |
| R3-184 | 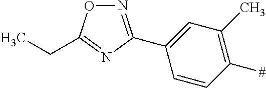 |
| R3-185 | 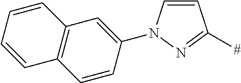 |
| R3-186 | 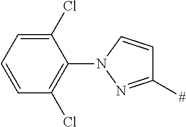 |
| R3-187 | 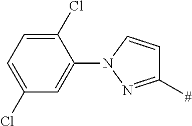 |
| R3-188 | 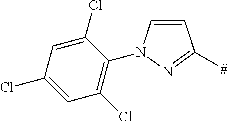 |
| R3-189 | 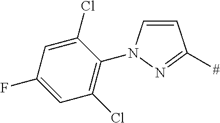 |
| R3-190 | 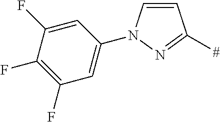 |
| R3-191 | 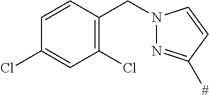 |
| R3-192 | 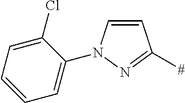 |
| R3-193 | 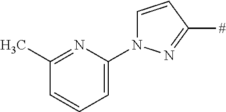 |
| R3-194 | 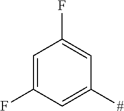 |
| R3-195 | 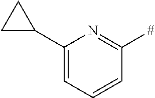 |
| R3-196 | 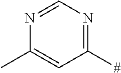 |
| R3-197 | 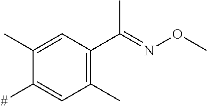 |
| R3-198 | 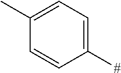 |
| R3-199 | 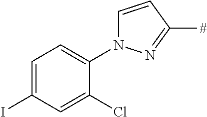 |
| R3-200 | 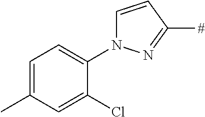 |
| R3-201 | 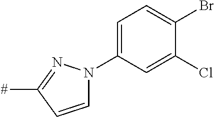 |
| R3-202 | 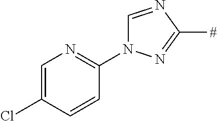 |
| R3-203 | 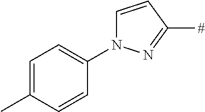 |
| R3-204 | 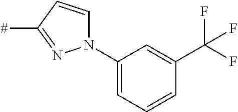 |
| R3-205 | 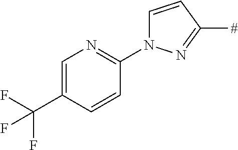 |
| R3-206 | 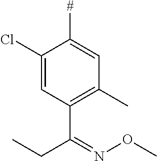 |
| R3-207 | 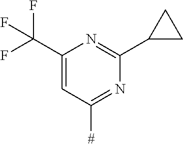 |
| R3-208 | 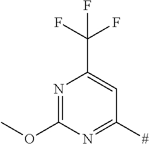 |
| R3-209 | 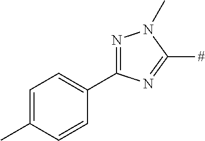 |
| R3-210 | 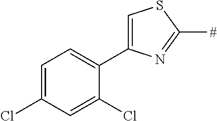 |
| R3-211 | 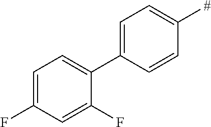 |
| R3-212 | 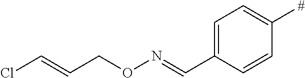 |
| R3-213 | 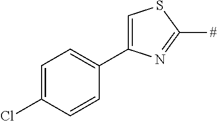 |
| R3-214 | 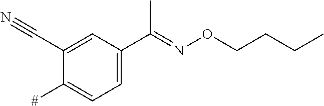 |
| R3-215 | 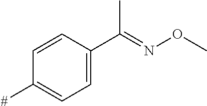 |
| R3-216 | 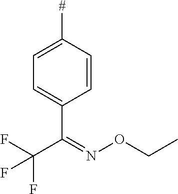 |
| R3-217 | 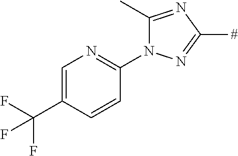 |
| R3-218 | 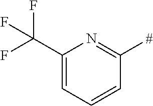 |
| R3-219 | 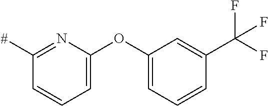 |
| R3-220 | 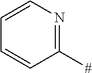 |
| R3-221 | 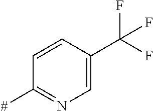 |
| R3-222 | 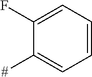 |
| R3-223 | 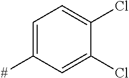 |
| R3-224 | 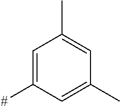 |
| R3-225 | 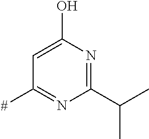 |

R^{3a} according to the invention is amino, halogen, hydroxy, nitro, cyano, carboxyl, C₁-C₆-alkyl, C₂-C₆-alkenyl, C₂-C₆-alkynyl, C₁-C₆-alkoxy, C₃-C₆-cycloalkyl, C₃-C₆-cycloalkenyl, C₂-C₆-alkenyloxy, C₃-C₆-alkynyloxy, C₁-C₆-alkoxyimino-C₁-C₄-alkyl, C₂-C₆-alkenyloxyimino-C₁-C₄-alkyl, C₃-C₆-alkynyloxyimino-C₁-C₄-alkyl, C₁-C₆-alkylamino, C(═O)—(C₁-C₆-alkyl), C(═O)—(C₁-C₆-alkoxy), phenyl, naphthyl or a 3- to 10-membered saturated, partially unsaturated or aromatic mono- or bicyclic heterocycle, wherein the ring member atoms of the heterocycle include beside carbon atoms 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the aforementioned phenyl and heterocycle groups R^{3a} are attached to R³ via a direct bond, an oxygen or sulfur atom, the latter two atoms forming a linker between said residues; and wherein the aliphatic or cyclic groups R^{3a} are unsubstituted or substituted by 1, 2 or 3 or up to the maximum possible number of identical or different groups R^{3b} as defined or preferably defined below; in particular R^{3b} is CH₃, OCH₃, SCF₃, cyano, F or Cl. In one embodiment of the invention R^{3a} is halogen, C₁-C₆-alkyl, C₂-C₆-alkenyl, C₁-C₆-alkoxy, C₁-C₆-alkoxyimino-C₁-C₄-alkyl, C₂-C₆-alkenyloxyimino-C₁-C₄-alkyl, C₃-C₆-alkynyloxyimino-C₁-C₄-alkyl, phenyl or a 5- or 6-membered saturated, partially unsaturated or aromatic heterocycle, which, in addition to carbon atoms, contains as ring members 1, 2 or 3 heteroatoms independently selected from N, O and S as ring member atoms; and wherein the aliphatic or cyclic groups R^{3a} are unsubstituted or substituted by 1, 2 or 3 or up to the maximum possible number of identical or different groups R^{3b} as defined or preferably defined below; in particular R^{3b} is CH₃, OCH₃, SCF₃, cyano, F or Cl.

In another embodiment R^{3a} is a 5- or 6-membered saturated, partially unsaturated or aromatic heterocycle, which, in addition to carbon atoms, contains as ring members 1, 2 or 3 heteroatoms independently selected from N, O and S as ring member atoms; and wherein the aforementioned heterocyclic groups R^{3a} are attached via a direct bond, an oxygen or sulfur atom, the latter two atoms forming a linker between said residues; and wherein the aliphatic or cyclic groups R^{3a} are unsubstituted or substituted by 1, 2 or 3 or up to the maximum possible number of identical or different groups R^{3b} as defined or preferably defined below; in particular R^{3b} is CH₃, OCH₃, SCF₃, cyano, F or Cl.

In a further embodiment R^{3a} is halogen, cyano, C₁-C₆-alkyl, C₁-C₆-haloalkyl, C₁-C₆-alkoxy, C₁-C₆-haloalkoxy, C₂-C₆-alkenyloxyimino-C₁-C₄-alkyl or C₃-C₆-alkynyloxyimino-C₁-C₄-alkyl; and wherein the aliphatic groups R^{3a} are unsubstituted or substituted by 1, 2 or 3 or up to the maximum possible number of identical or different groups R^{3b} as defined or preferably defined below; in particular R^{3b} is CH₃, OCH₃, SCF₃, cyano, F or Cl.

In still another embodiment of the invention R^{3a} is C₁-C₄-alkoxyimino-C₁-C₄-alkyl, halogen, cyano, C₁-C₆-alkyl, C₂-C₆-alkenyl, C₁-C₆-alkoxy, C₃-C₆-cycloalkyl or C₃-C₆-cycloalkoxy. In a further embodiment R^{3a} is halogen, cyano, C₁-C₆-alkyl or C₁-C₆-haloalkylthio. In a further embodiment R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, SCF₃, SCHF₂, cyano, Cl, F or Br; in particular R^{3a} is F, Cl, SCF₃, cyano or CH₃.

In another aspect R^{3a} is phenyl, which is unsubstituted or substituted by 1, 2, 3 or up to the maximum possible number of identical or different groups R^{3b} as defined or preferably defined below; in particular R^{3b} is CH₃, OCH₃, SCF₃, cyano, F or Cl;

In a further aspect R^{3a} is phenyl and is attached to a 5-membered aromatic heterocycle R³ in a 1,3-substitution pattern relative to the group L, i.e. attached to ring member atoms of the heterocycle which are not adjacent to one another; wherein said group R^{3a} is unsubstituted or substituted by 1, 2 or 3 identical or different groups R^{3b} as defined or preferably defined below; in particular R^{3b} is CH₃, OCH₃, SCF₃, cyano, F or Cl.

In yet another embodiment R^{3a} is phenyl and is attached to a 6-membered aromatic carbo- or heterocycle R³ in a 1,4-substitution pattern relative to the group L, i.e. attached to opposite ring member atoms of said aromatic carbo- or heterocycle; wherein said group R^{3a} is unsubstituted or substituted by 1, 2 or 3 identical or different groups R^{3b} as defined or preferably defined below; in particular R^{3b} is CH₃, OCH₃, SCF₃, cyano, F or Cl.

R^{3b} according to the invention is halogen, hydroxy, nitro, cyano, carboxyl, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₁-C₆-alkoxyimino-C₁-C₄-alkyl, C₂-C₆-alkenyloxyimino-C₁-C₄-alkyl, C₃-C₆-alkynyloxyimino-C₁-C₄-alkyl, C₁-C₆-alkylthio, C₁-C₆-alkylsulfinyl, C₁-C₆-alkylsulfonyl, C₃-C₆-cycloalkyl, C₃-C₆-cycloalkenyl, phenyl or a 5- or 6-membered saturated, partially unsaturated or aromatic heterocycle; wherein the ring member atoms of the heterocycle include beside carbon atoms 1, 2 or 3 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the aforementioned cyclic groups R^{3b} are attached to R^{3a} via a direct bond, an oxygen or sulfur atom, the latter two atoms forming a linker between said residues; and wherein the aliphatic or cyclic groups R^{3b} are unsubstituted or substituted by 1, 2 or 3 or up to the maximum possible number of identical or different groups selected from halogen, C₁-C₆-alkyl, C₁-C₆-haloalkyl and C₃-C₆-cycloalkyl. In one embodiment of the invention R^{3b} is halogen, cyano, C₁-C₆-alkyl, C₁-C₆-alkoxy, C₂-C₆-alkenyl, C₂-C₆-alkenyloxy, C₁-C₆-alkylthio, phenyl or a 5- or 6-membered saturated, partially unsaturated or aromatic heterocycle, which, in addition to carbon atoms, contains 1, 2 or 3 heteroatoms independently selected from N, O and S as ring member atoms; wherein the aforementioned cyclic groups R^{3b} are unsubstituted or substituted by 1, 2, 3 or up to the maximum possible number of identical or different groups selected from halogen, C₁-C₆-alkyl, C₁-C₆-haloalkyl and C₃-C₆-cycloalkyl.

In another embodiment R^{3b} is halogen, C₁-C₆-alkyl, C₁-C₆-haloalkyl, C₁-C₆-alkoxy, C₁-C₆-haloalkoxy, C₁-C₆-alkylthio or C₁-C₆-haloalkylthio; more preferably F or Cl; in particular F. In yet another embodiment R^{3b} is phenyl, which is unsubstituted or substituted by 1, 2, 3 or up to the maximum possible number of identical or different groups selected from halogen, C₁-C₆-alkyl and C₁-C₆-haloalkyl.

In a further embodiment R^{3b} is a 5- or 6-membered aromatic heterocycle, which, in addition to carbon atoms, contains 1, 2 or 3 heteroatoms independently selected from N, O and S as ring member atoms; wherein the aforementioned cyclic groups R^{3b} are unsubstituted or substituted by 1, 2, 3 or up to the maximum possible number of identical or different groups selected from halogen, C₁-C₆-alkyl and C₁-C₆-haloalkyl.

Q according to the invention is a divalent group selected from —O—, —S—, —(NQ^{a})-, —(CQ^{b}Q^{c})-, —(C(═N—O-Q^{a})-, —(C═O)— and —(C═S)—; wherein Q^{a}, Q^{b} and Q^{c} are as defined or preferably defined below; in particular Q^{a} is hydrogen, CH₃ or CH₂CH₃ and Q^{b} and Q^{c} are independently selected from hydrogen, halogen, CH₃ and CH₂CH₃. In one embodiment of the invention Q is a divalent group selected from —O—, —(NQ^{a})-, —(CQ^{b}Q^{c})- and —(C(═N—O-Q^{a})-)-; wherein Q^{a}, Q^{b} and Q^{c} are as defined or preferably defined below; in particular Q^{a} is hydrogen, CH₃ or CH₂CH₃ and Q^{b} and Q^{c} are independently selected from hydrogen, halogen, CH₃ and CH₂CH₃. In another embodiment Q is a divalent group selected from —O—, —(NQ^{a})- and —(CQ^{b}Q^{c})-)- wherein Q^{a}, Q^{b} and Q^{c} are as defined or preferably defined below; in particular Q^{a} is hydrogen, CH₃ or CH₂CH₃ and Q^{b} and Q^{c} are independently selected from hydrogen, halogen, CH₃ and CH₂CH₃. In a preferred embodiment Q is a divalent group selected from —(NQ^{a})- and —(CQ^{b}Q^{c})-)-; wherein Q^{a}, Q^{b} and Q^{c} are as defined or preferably defined below; in particular Q^{a} is hydrogen, CH₃ or CH₂CH₃ and Q^{b} and Q^{c} are independently selected from hydrogen, halogen, CH₃ and CH₂CH₃. In another preferred embodiment Q is —(NQ^{a})-)-; wherein Q^{a} is as defined or preferably defined below; in particular Q^{a} is hydrogen, CH₃ or CH₂CH₃. In still another preferred embodiment Q is a divalent group —(CQ^{b}Q^{c})-)-; wherein Q^{b} and Q^{c} are as defined or preferably defined below; in particular Q^{b} and Q^{c} are independently selected from hydrogen, halogen, CH₃ and CH₂CH₃.

In a more preferred embodiment Q is a divalent group selected from —CH₂—, —NH— and —NCH₃—. Q^{a} according to the invention is hydrogen, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl, phenyl-C₁-C₄-alkyl, heteroaryl-C₁-C₄-alkyl or C₃-C₆-cycloalkyl-C₁-C₄-alkyl; wherein the aliphatic, alicyclic and aromatic moieties of Q^{a} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy.

In one preferred embodiment of the invention Q^{a} is hydrogen, C₁-C₆-alkyl, C₂-C₆-alkenyl, C₂-C₆-alkynyl or C₃-C₆-cycloalkyl; wherein the aliphatic and alicyclic moieties of Q^{a} are unsubstituted or substituted by 1, 2, 3 or up to the maximum number of identical or different groups selected from halogen, cyano, C₁-C₄-alkyl and C₁-C₄-alkoxy.

In another preferred embodiment Q^{a} is hydrogen, C₁-C₆-alkyl, C₂-C₆-alkenyl, C₂-C₆-alkynyl or C₃-C₆-cycloalkyl. Preferrably Q^{a} is hydrogen or C₁-C₆-alkyl, in particular hydrogen, CH₃ or CH₂CH₃. In a further preferred embodiment Q^{a} is hydrogen.

In still another preferred embodiment Q^{a} is hydrogen, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl, heteroaryl-C₁-C₄-alkyl or C₃-C₆-cycloalkyl-C₁-C₄-alkyl; wherein the aliphatic, alicylic and aromatic moieties of Q^{a} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy.

Q^{b}, Q^{c} according to the invention are independently selected from hydrogen, halogen, cyano, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl and C₃-C₆-cycloalkyl-C₁-C₄-alkyl; wherein the aliphatic and alicyclic moieties of Q^{b} and/or Q^{c} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy; or Q^{b} and Q^{c} together with the carbon atom to which they are bound form a saturated or partially unsaturated 3-, 4-, 5-, 6- or 7-membered carbocycle or a saturated or partially unsaturated 3-, 4-, 5-, 6- or 7-membered heterocycle, wherein the heterocycle includes beside carbon atoms 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the carbo- and heterocycle are unsubstituted or substituted by 1, 2, 3 or 4 identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy.

In a preferred embodiment of the invention Q^{b} and Q^{c} are independently selected from hydrogen, halogen, C₁-C₆-alkyl, C₁-C₆-alkoxy, C₂-C₆-alkenyl, C₂-C₆-alkynyl and C₃-C₆-cycloalkyl; wherein the aliphatic and alicyclic moieties of Q^{b} and/or Q^{c} are unsubstituted or substituted by 1, 2, 3 or up to the maximum number of identical or different groups selected from halogen, cyano, C₁-C₄-alkyl and C₁-C₄-alkoxy; or Q^{b} and Q^{c} together with the carbon atom to which they are bound form a saturated or partially unsaturated 3-, 4- or 5-membered carbocycle or a saturated or partially unsaturated 3-, 4- or 5-membered heterocycle, wherein the heterocycle includes beside carbon atoms 1, 2, 3 or 4 heteroatoms independently selected from N, O and S as ring member atoms; and wherein the carbo- and heterocycle are unsubstituted or substituted by 1, 2, 3 or 4 identical or different groups selected from halogen, cyano, C₁-C₄-alkyl and C₁-C₄-alkoxy.

In another preferred embodiment Q^{b} and Q^{c} are independently selected from hydrogen, halogen, C₁-C₆-alkyl, C₁-C₆-haloalkyl, C₁-C₆-alkoxy, C₁-C₆-haloalkoxy, C₂-C₆-alkenyl, C₂-C₆-haloalkenyl, C₃-C₆-cycloalkyl and C₃-C₆-halocycloalkyl.

In a further preferred embodiment Q^{b} and Q^{c} are independently selected from hydrogen, halogen and C₁-C₆-alkyl, in particular Q^{b} and Q^{c} are independently selected from hydrogen, halogen, CH₃ and CH₂CH₃.

In a preferred embodiment Q^{b} and Q^{c} are independently selected from hydrogen and F.

In another aspect of the invention Q^{b} and Q^{c} together with the carbon atom to which they are bound form a saturated or partially unsaturated 3-, 4- or 5-membered carbocycle or a saturated or partially unsaturated 3-, 4- or 5-membered heterocycle, wherein the heterocycle includes beside carbon atoms 1 or 2 heteroatoms independently selected from N, O and S as ring member atoms; and wherein the carbo- and heterocycle are unsubstituted or substituted by 1, 2, 3 or 4 identical or different groups independently selected from halogen, cyano, C₁-C₄-alkyl and C₁-C₄-alkoxy.

In a further preferred aspect of the invention Q^{b} and Q^{c} together with the carbon atom to which they are bound form a cyclopropane, cyclobutane, cyclopentane, aziridine, thiirane, oxirane or oxetane ring. In one preferred embodiment Q^{b} and Q^{c} together with the carbon atom to which they are bound form a cyclopropane or oxirane ring.

W according to the invention is O or S. In a preferred embodiment W is O.

Y according to the invention is a divalent group selected from —O—, —S— and —NY^{a}—; wherein Y^{a} is as defined or preferably defined below; in particular Y^{a} is hydrogen, CH₃ or CH₂CH₃. In a preferred embodiment of the invention Y is a divalent group selected from —O— and —NY^{a}—, wherein Y^{a} is as defined or preferably defined below; in particular Y^{a} is hydrogen, CH₃ or CH₂CH₃. In another preferred embodiment of the invention Y is —O—. In yet another preferred embodiment of the invention Y is —NY^{a}—, wherein Y^{a} is as defined or preferably defined below; in particular Y^{a} is hydrogen, CH₃ or CH₂CH₃.

Y^{a} according to the invention is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, phenyl-C₁-C₄-alkyl, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy. In a preferred embodiment Y^{a} is hydrogen, C₁-C₆-alkyl, C₁-C₆-alkoxy, C₂-C₆-alkenyl, C₂-C₆-alkynyl or C₃-C₆-cycloalkyl. In another preferred embodiment Y^{a} is hydrogen, C₁-C₆-alkyl, C₁-C₆-alkoxy or C₃-C₆-cycloalkyl. In a further preferred embodiment Y^{a} is hydrogen, CH₃ or CH₂CH₃.

In another preferred embodiment, Y^{a} is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy. In still another preferred embodiment, Y^{a} is hydrogen, CH₃, CH₂CH₃, CF₃, CHF₂, OCF₃, OCHF₂, OCF₃, F, Cl, and Br.

In a further preferred embodiment, Y^{a} and Q^{a} are independently selected from hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy.

In a still further preferred embodiment, Y^{a} and R^{x} are independently selected from hydrogen, CH₃, CH₂CH₃, CF₃, CHF₂, OCF₃, OCHF₂, OCF₃, F, Cl, and Br.

R^{V} and R^{Y} according to the invention together with the group —(N—C(═W)—Y)—, form a 5-, 6- or 7-membered saturated heterocycle; wherein the heterocycle includes beside carbon atoms 1, 2, 3 or 4 heteroatoms independently selected from N, O and S as ring member atoms; and wherein 1 or 2 carbon ring member atoms of the heterocycle may be replaced by 1 or 2 groups independently selected from C(═O) and C(═S); and wherein the heterocycle is unsubstituted or substituted by 1, 2, 3 or 4 identical or different groups R^{X} as defined or preferably defined below.

In a preferred embodiment R^{V} and R^{Y} together with the group —(N—C(═W)—Y)—, form a 5- or 6-membered saturated heterocycle; wherein the heterocycle includes beside carbon atoms 1, 2 or 3 heteroatoms independently selected from N, O and S as ring member atoms; and wherein 1 or 2 carbon ring member atoms of the heterocycle may be replaced by 1 or 2 groups independently selected from C(═O) and C(═S); and wherein the heterocycle is unsubstituted or substituted by 1, 2, 3 or 4 identical or different groups R^{X} as defined or preferably defined below; in particular R^{X} is hydrogen, CH₃, CH₂CH₃ or OCH₃. In another preferred embodiment R^{V} and R^{Y} together with the group —(N—C(═W)—Y)—, form a 5-membered saturated heterocycle; wherein the heterocycle includes beside carbon atoms 1, 2 or 3 heteroatoms independently selected from N, O and S as ring member atoms; and wherein 1 or 2 carbon ring member atoms of the heterocycle may be replaced by 1 or 2 groups independently selected from C(═O) and C(═S); and wherein the heterocycle is unsubstituted or substituted by 1, 2, 3 or 4 identical or different groups R^{X} as defined or preferably defined below; in particular R^{X} is hydrogen, CH₃, CH₂CH₃ or OCH₃.

In another preferred embodiment, R^{x} is hydrogen, CH₃, CH₂CH₃, CF₃, CHF₂, OCF₃, OCHF₂, OCF₃, F, Cl, or Br.

In one preferred aspect of the invention R^{V} and R^{Y} together with the group —(N—C(═W)—Y)—, form a heterocycle C.1 to C.6, wherein # denotes the point of attachment to the group Q; and wherein W is O, further preferably a heterocycle C.1 or C.4, even more preferably the heterocycle C.1. In a more preferred embodiment R^{V} and R^{Y} together with the group —(N—C(═W)—Y)—, form a heterocycle C.1 to C.3; and wherein W is O.

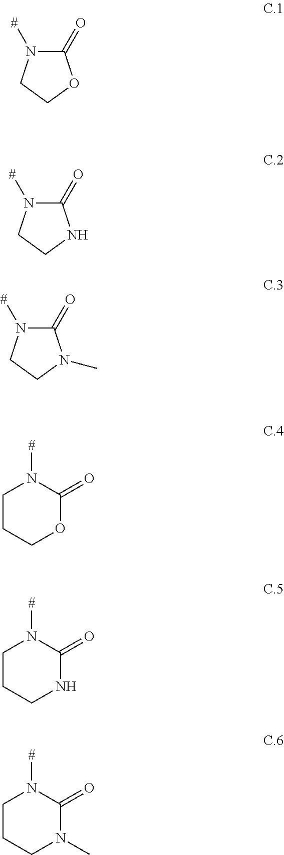

In another embodiment, when Q is C(═O), R^{V} and R^{Y} together with the group —(N—C(═W)—Y)— do not form the heterocycle C.7:

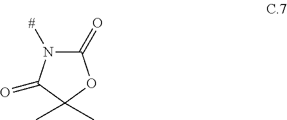

R^{X} according to the invention is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, C₁-C₆-alkylamino, C₁-C₆-dialkylamino or C₃-C₆-cycloalkylamino; wherein the aliphatic and alicyclic moieties of R^{X} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy. In a preferred embodiment R^{X} is hydrogen, C₁-C₆-alkyl, C₁-C₆-alkoxy, C₂-C₆-alkenyl, C₂-C₆-alkynyl or C₃-C₆-cycloalkyl. In another preferred embodiment R^{X} is hydrogen, C₁-C₆-alkyl or C₁-C₆-alkoxy. In still another preferred embodiment R^{X} is hydrogen, CH₃, CH₂CH₃ or OCH₃.

In another embodiment, the compound of formula I is not

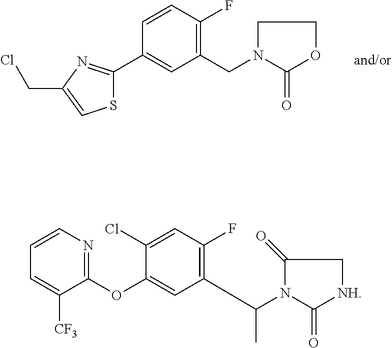 [SMILES: O=C1OCCN1Cc1cc(-c2nc(CCl)cs2)ccc1F ; CC(c1cc(Oc2ncccc2C(F)(F)F)c(Cl)cc1F)N1C(=O)CNC1=O]

In a further preferred embodiment the invention relates to compounds of formula I.A, wherein R³ is 1-phenylpyrazol-3-yl, r is 0, n is 1, 2 or 3 and L is —OCH₂—.

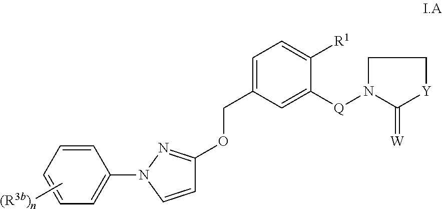

In a further preferred embodiment the invention relates to compounds of formula I.B, wherein R³ is 1-phenylpyrazol-3-yl, r is 0, n is 1, 2 or 3 and L is —CH₂—.

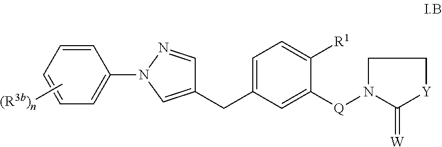

In a further preferred embodiment the invention relates to compounds of formula I.C, wherein r is 0, R³ is 2-pyridinyl, which is substituted by a group R^{3a} in 6-position and wherein L is —CH₂O—N═C(CH₃)—.

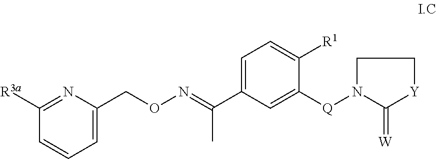

In a further preferred embodiment the invention relates to compounds of formula I wherein the meaning of R¹, Q and the meaning of R^{V} and R^{Y}, which, together with the group —(N—C(═W)—Y)—, form a heterocycle C.1, C.2, C.3 or C.4 (W being O), in each case is one of the following combinations in lines B-1 to B-60 in Table B; wherein Me stands for CH₃ or methyl.

TABLE B

| Line | R¹ | Q | R^{V}/R^{Y} |
|---|---|---|---|
| B-1 | F | —CH₂— | C.1 |
| B-2 | Cl | —CH₂— | C.1 |
| B-3 | Me | —CH₂— | C.1 |
| B-4 | OMe | —CH₂— | C.1 |
| B-5 | cyano | —CH₂— | C.1 |
| B-6 | F | —NH— | C.1 |
| B-7 | Cl | —NH— | C.1 |
| B-8 | Me | —NH— | C.1 |
| B-9 | OMe | —NH— | C.1 |
| B-10 | cyano | —NH— | C.1 |
| B-11 | F | —NMe— | C.1 |
| B-12 | Cl | —NMe— | C.1 |
| B-13 | Me | —NMe— | C.1 |
| B-14 | OMe | —NMe— | C.1 |
| B-15 | cyano | —NMe— | C.1 |
| B-16 | F | —CH₂— | C.2 |
| B-17 | Cl | —CH₂— | C.2 |
| B-18 | Me | —CH₂— | C.2 |
| B-19 | OMe | —CH₂— | C.2 |
| B-20 | cyano | —CH₂— | C.2 |
| B-21 | F | —NH— | C.2 |
| B-22 | Cl | —NH— | C.2 |
| B-23 | Me | —NH— | C.2 |
| B-24 | OMe | —NH— | C.2 |
| B-25 | cyano | —NH— | C.2 |
| B-26 | F | —NMe— | C.2 |
| B-27 | Cl | —NMe— | C.2 |
| B-28 | Me | —NMe— | C.2 |
| B-29 | OMe | —NMe— | C.2 |
| B-30 | cyano | —NMe— | C.2 |
| B-31 | F | —CH₂— | C.3 |
| B-32 | Cl | —CH₂— | C.3 |
| B-33 | Me | —CH₂— | C.3 |
| B-34 | OMe | —CH₂— | C.3 |
| B-35 | cyano | —CH₂— | C.3 |
| B-36 | F | —NH— | C.3 |
| B-37 | Cl | —NH— | C.3 |
| B-38 | Me | —NH— | C.3 |
| B-39 | OMe | —NH— | C.3 |
| B-40 | cyano | —NH— | C.3 |
| B-41 | F | —NMe— | C.3 |
| B-42 | Cl | —NMe— | C.3 |
| B-43 | Me | —NMe— | C.3 |
| B-44 | OMe | —NMe— | C.3 |
| B-45 | cyano | —NMe— | C.3 |
| B-46 | F | —CH₂— | C.4 |
| B-47 | Cl | —CH₂— | C.4 |
| B-48 | Me | —CH₂— | C.4 |
| B-49 | OMe | —CH₂— | C.4 |
| B-50 | cyano | —CH₂— | C.4 |
| B-51 | F | —NH— | C.4 |
| B-52 | Cl | —NH— | C.4 |
| B-53 | Me | —NH— | C.4 |
| B-54 | OMe | —NH— | C.4 |
| B-55 | cyano | —NH— | C.4 |
| B-56 | F | —NMe— | C.4 |
| B-57 | Cl | —NMe— | C.4 |
| B-58 | Me | —NMe— | C.4 |
| B-59 | OMe | —NMe— | C.4 |
| B-60 | cyano | —NMe— | C.4 |

Particularly preferred compounds I according to the present invention are compounds I.1 to I.9:
I.1: 3-[[2-chloro-5-[[1-(4-chlorophenyl)pyrazol-3-yl]oxymethyl]phenyl]methyl]oxazolidin-2-one
I.2: 3-[2-chloro-5-[[1-(4-chlorophenyl)pyrazol-3-yl]oxymethyl]anilino]oxazolidin-2-one
I.3: 1-[[2-chloro-5-[[1-(4-chlorophenyl)pyrazol-3-yl]oxymethyl]phenyl]methyl]-3-methyl-imidazolidin-2-one
I.4: 3-[[2-chloro-5-[(E)-C-methyl-N-[(6-methyl-2-pyridyl)methoxy]carbonimidoyl]phenyl]methyl]oxazolidin-2-one
I.5: 3-[2-chloro-5-[(E)-C-methyl-N-[(6-methyl-2-pyridyl)methoxy]carbonimidoyl]anilino]oxazolidin-2-one
I.6: 1-[[2-chloro-5-[(E)-C-methyl-N-[(6-methyl-2-pyridyl)methoxy]carbonimidoyl]phenyl]methyl]-3-methyl-imidazolidin-2-one
I.7: 3-[[2-chloro-5-[(E)-C-methyl-N-[[6-(trifluoromethyl)-2-pyridyl]methoxy]carbonimidoyl]phenyl]methyl]oxazolidin-2-one
I.8: 3-[2-chloro-5-[(E)-C-methyl-N-[[6-(trifluoromethyl)-2-pyridyl]methoxy]carbonimidoyl]anilino]oxazolidin-2-one
I.9: 1-[[2-chloro-5-[(E)-C-methyl-N-[[6-(trifluoromethyl)-2-pyridyl]methoxy]carbonimidoyl]phenyl]methyl]-3-methyl-imidazolidin-2-one

With respect to their use, particular preference is given to the compounds compiled in the Tables 1 to 180 below, wherein the meaning of R³ in each case is selected from groups R3-1 to R3-225 in Table A and wherein the meaning of the combination of substituents R¹, Q and the groups R^{V} and R^{Y}, together forming a heterocyclic group C.1, C.2, C.3 or C.4 as defined herein (W being O), is selected from lines B-1 to B-60 as described in Table B.

Table 1: Compounds I wherein L is —OCH₂—, r is 0 and wherein the meaning of R³ is selected in each case from any one of the groups R3-1 to R3-225 in Table A; and wherein the combination of substituents R¹, Q and R^{V}/R^{Y} corresponds to line B-1 in Table B.

Table 2: Compounds I wherein L, r and R³ are as defined in Table 1; and wherein the combination of substituents R¹, Q and R^{V}/R^{Y} corresponds to line B-2 in Table B.

Table 3: Compounds I wherein L, r and R³ are as defined in Table 1; and wherein the combination of substituents R¹, Q and R^{V}/R^{Y} corresponds to line B-3 in Table B.

Table 4: Compounds I wherein L, r and R³ are as defined in Table 1; and wherein the combination of substituents R¹, Q and R^{V}/R^{Y} corresponds to line B-4 in Table B.

Table 5: Compounds I wherein L, r and R³ are as defined in Table 1; and wherein the combination of substituents R¹, Q and R^{V}/R^{Y} corresponds to line B-5 in Table B.

Table 6: Compounds I wherein L, r and R³ are as defined in Table 1; and wherein the combination of substituents R¹, Q and R^{V}/R^{Y} corresponds to line B-6 in Table B.

Table 7: Compounds I wherein L, r and R³ are as defined in Table 1; and wherein the combination of substituents R¹, Q and R^{V}/R^{Y} corresponds to line B-7 in Table B.

Table 8: Compounds I wherein L, r and R³ are as defined in Table 1; and wherein the combination of substituents R¹, Q and R^{V}/R^{Y} corresponds to line B-8 in Table B.

Table 9: Compounds I wherein L, r and R³ are as defined in Table 1; and wherein the combination of substituents R¹, Q and R^{V}/R^{Y} corresponds to line B-9 in Table B.

Table 10: Compounds I wherein L, r and R³ are as defined in Table 1; and wherein the combination of substituents R¹, Q and R^{V}/R^{Y} corresponds to line B-10 in Table B.

Table 11: Compounds I wherein L, r and R³ are as defined in Table 1; and wherein the combination of substituents R¹, Q and R^{V}/R^{Y} corresponds to line B-11 in Table B.

Table 12: Compounds I wherein L, r and R³ are as defined in Table 1; and wherein the combination of substituents R¹, Q and R^{V}/R^{Y} corresponds to line B-12 in Table B.

Table 13: Compounds I wherein L, r and R³ are as defined in Table 1; and wherein the combination of substituents R¹, Q and R^{V}/R^{Y} corresponds to line B-13 in Table B.

Table 14: Compounds I wherein L, r and R³ are as defined in Table 1; and wherein the combination of substituents R¹, Q and R^{V}/R^{Y} corresponds to line B-14 in Table B.

Table 15: Compounds I wherein L, r and R³ are as defined in Table 1; and wherein the combination of substituents R¹, Q and R^{V}/R^{Y} corresponds to line B-15 in Table B.

Table 16: Compounds I wherein L, r and R³ are as defined in Table 1; and wherein the combination of substituents R¹, Q and R^{V}/R^{Y} corresponds to line B-16 in Table B.

Table 17: Compounds I wherein L, r and R³ are as defined in Table 1; and wherein the combination of substituents R¹, Q and R^{V}/R^{Y} corresponds to line B-17 in Table B.

Table 18: Compounds I wherein L, r and R³ are as defined in Table 1; and wherein the combination of substituents R¹, Q and R^{V}/R^{Y} corresponds to line B-18 in Table B.

Table 19: Compounds I wherein L, r and R³ are as defined in Table 1; and wherein the combination of substituents R¹, Q and R^{V}/R^{Y} corresponds to line B-19 in Table B.

Table 20: Compounds I wherein L, r and R³ are as defined in Table 1; and wherein the combination of substituents R¹, Q and R^{V}/R^{Y} corresponds to line B-20 in Table B.

Table 21: Compounds I wherein L, r and R³ are as defined in Table 1; and wherein the combination of substituents R¹, Q and R^{V}/R^{Y} corresponds to line B-21 in Table B.

Table 22: Compounds I wherein L, r and R³ are as defined in Table 1; and wherein the combination of substituents R¹, Q and R^{V}/R^{Y} corresponds to line B-22 in Table B.

Table 23: Compounds I wherein L, r and R³ are as defined in Table 1; and wherein the combination of substituents R¹, Q and R^{V}/R^{Y} corresponds to line B-23 in Table B.

Table 24: Compounds I wherein L, r and R³ are as defined in Table 1; and wherein the combination of substituents R¹, Q and R^{V}/R^{Y} corresponds to line B-24 in Table B.

Table 25: Compounds I wherein L, r and R³ are as defined in Table 1; and wherein the combination of substituents R¹, Q and R^{V}/R^{Y} corresponds to line B-25 in Table B.

Table 26: Compounds I wherein L, r and R³ are as defined in Table 1; and wherein the combination of substituents R¹, Q and R^{V}/R^{Y} corresponds to line B-26 in Table B.

Table 27: Compounds I wherein L, r and R³ are as defined in Table 1; and wherein the combination of substituents R¹, Q and R^{V}/R^{Y} corresponds to line B-27 in Table B.

Table 28: Compounds I wherein L, r and R³ are as defined in Table 1; and wherein the combination of substituents R¹, Q and R^{V}/R^{Y} corresponds to line B-28 in Table B.

Table 29: Compounds I wherein L, r and R³ are as defined in Table 1; and wherein the combination of substituents R¹, Q and R^{V}/R^{Y} corresponds to line B-29 in Table B.

Table 30: Compounds I wherein L, r and R³ are as defined in Table 1; and wherein the combination of substituents R¹, Q and R^{V}/R^{Y} corresponds to line B-30 in Table B.

Table 31: Compounds I wherein L, r and R³ are as defined in Table 1; and wherein the combination of substituents R¹, Q and R^{V}/R^{Y} corresponds to line B-31 in Table B.

Table 32: Compounds I wherein L, r and R³ are as defined in Table 1; and wherein the combination of substituents R¹, Q and R^{V}/R^{Y} corresponds to line B-32 in Table B.

Table 33: Compounds I wherein L, r and R³ are as defined in Table 1; and wherein the combination of substituents R¹, Q and R^{V}/R^{Y} corresponds to line B-33 in Table B.

Table 34: Compounds I wherein L, r and R³ are as defined in Table 1; and wherein the combination of substituents R¹, Q and R^{V}/R^{Y} corresponds to line B-34 in Table B.

Table 35: Compounds I wherein L, r and R³ are as defined in Table 1; and wherein the combination of substituents R¹, Q and R^{V}/R^{Y} corresponds to line B-35 in Table B.

Table 36: Compounds I wherein L, r and R³ are as defined in Table 1; and wherein the combination of substituents R¹, Q and R^{V}/R^{Y} corresponds to line B-36 in Table B.

Table 37: Compounds I wherein L, r and R³ are as defined in Table 1; and wherein the combination of substituents R¹, Q and R^{V}/R^{Y} corresponds to line B-37 in Table B.

Table 38: Compounds I wherein L, r and R³ are as defined in Table 1; and wherein the combination of substituents R¹, Q and R^{V}/R^{Y} corresponds to line B-38 in Table B.

Table 39: Compounds I wherein L, r and R³ are as defined in Table 1; and wherein the combination of substituents R¹, Q and R^{V}/R^{Y} corresponds to line B-39 in Table B.

Table 40: Compounds I wherein L, r and R³ are as defined in Table 1; and wherein the combination of substituents R¹, Q and R^{V}/R^{Y} corresponds to line B-40 in Table B.

Table 41: Compounds I wherein L, r and R³ are as defined in Table 1; and wherein the combination of substituents R¹, Q and R^{V}/R^{Y} corresponds to line B-41 in Table B.

Table 42: Compounds I wherein L, r and R³ are as defined in Table 1; and wherein the combination of substituents R¹, Q and R^{V}/R^{Y} corresponds to line B-42 in Table B.

Table 43: Compounds I wherein L, r and R³ are as defined in Table 1; and wherein the combination of substituents R¹, Q and R^{V}/R^{Y} corresponds to line B-43 in Table B.

Table 44: Compounds I wherein L, r and R³ are as defined in Table 1; and wherein the combination of substituents R¹, Q and R^{V}/R^{Y} corresponds to line B-44 in Table B.

Table 45: Compounds I wherein L, r and R³ are as defined in Table 1; and wherein the combination of substituents R¹, Q and R^{V}/R^{Y} corresponds to line B-45 in Table B.

Table 46: Compounds I wherein L, r and R³ are as defined in Table 1; and wherein the combination of substituents R¹, Q and R^{V}/R^{Y} corresponds to line B-46 in Table B.

Table 47: Compounds I wherein L, r and R³ are as defined in Table 1; and wherein the combination of substituents R¹, Q and R^{V}/R^{Y} corresponds to line B-47 in Table B.

Table 48: Compounds I wherein L, r and R³ are as defined in Table 1; and wherein the combination of substituents R¹, Q and R^{V}/R^{Y} corresponds to line B-48 in Table B.

Table 49: Compounds I wherein L, r and R³ are as defined in Table 1; and wherein the combination of substituents R¹, Q and R^{V}/R^{Y} corresponds to line B-49 in Table B.

Table 50: Compounds I wherein L, r and R³ are as defined in Table 1; and wherein the combination of substituents R¹, Q and R^{V}/R^{Y} corresponds to line B-50 in Table B.

Table 51: Compounds I wherein L, r and R³ are as defined in Table 1; and wherein the combination of substituents R¹, Q and R^{V}/R^{Y} corresponds to line B-51 in Table B.

Table 52: Compounds I wherein L, r and R³ are as defined in Table 1; and wherein the combination of substituents R¹, Q and R^{V}/R^{Y} corresponds to line B-52 in Table B.

Table 53: Compounds I wherein L, r and R³ are as defined in Table 1; and wherein the combination of substituents R¹, Q and R^{V}/R^{Y} corresponds to line B-53 in Table B.

Table 54: Compounds I wherein L, r and R³ are as defined in Table 1; and wherein the combination of substituents R¹, Q and R^{V}/R^{Y} corresponds to line B-54 in Table B.

Table 55: Compounds I wherein L, r and R³ are as defined in Table 1; and wherein the combination of substituents R¹, Q and R^{V}/R^{Y} corresponds to line B-55 in Table B.

Table 56: Compounds I wherein L, r and R³ are as defined in Table 1; and wherein the combination of substituents R¹, Q and R^{V}/R^{Y} corresponds to line B-56 in Table B.

Table 57: Compounds I wherein L, r and R³ are as defined in Table 1; and wherein the combination of substituents R¹, Q and R^{V}/R^{Y} corresponds to line B-57 in Table B.

Table 58: Compounds I wherein L, r and R³ are as defined in Table 1; and wherein the combination of substituents R¹, Q and R^{V}/R^{Y} corresponds to line B-58 in Table B.

Table 59: Compounds I wherein L, r and R³ are as defined in Table 1; and wherein the combination of substituents R¹, Q and R^{V}/R^{Y} corresponds to line B-59 in Table B.

Table 60: Compounds I wherein L, r and R³ are as defined in Table 1; and wherein the combination of substituents R¹, Q and R^{V}/R^{Y} corresponds to line B-60 in Table B.

Table 61: Compounds I wherein L is —CH₂—, r is 0 and wherein the meaning of R³ is selected in each case from any one of the groups R3-1 to R3-225 in Table A; and wherein the combination of substituents R¹, Q and R^{V}/R^{Y} corresponds to line B-1 in Table B.

Table 62: Compounds I wherein L, r and R³ are as defined in Table 61; and wherein the combination of substituents R¹, Q and R^{V}/R^{Y} corresponds to line B-2 in Table B.

Table 63: Compounds I wherein L, r and R³ are as defined in Table 61; and wherein the combination of substituents R¹, Q and R^{V}/R^{Y} corresponds to line B-3 in Table B.

Table 64: Compounds I wherein L, r and R³ are as defined in Table 61; and wherein the combination of substituents R¹, Q and R^{V}/R^{Y} corresponds to line B-4 in Table B.

Table 65: Compounds I wherein L, r and R³ are as defined in Table 61; and wherein the combination of substituents R¹, Q and R^{V}/R^{Y} corresponds to line B-5 in Table B.

Table 66: Compounds I wherein L, r and R³ are as defined in Table 61; and wherein the combination of substituents R¹, Q and R^{V}/R^{Y} corresponds to line B-6 in Table B.

Table 67: Compounds I wherein L, r and R³ are as defined in Table 61; and wherein the combination of substituents R¹, Q and R^{V}/R^{Y} corresponds to line B-7 in Table B.

Table 68: Compounds I wherein L, r and R³ are as defined in Table 61; and wherein the combination of substituents R¹, Q and R^{V}/R^{Y} corresponds to line B-8 in Table B.

Table 69: Compounds I wherein L, r and R³ are as defined in Table 61; and wherein the combination of substituents R¹, Q and R^{V}/R^{Y} corresponds to line B-9 in Table B.

Table 70: Compounds I wherein L, r and R³ are as defined in Table 61; and wherein the combination of substituents R¹, Q and R^{V}/R^{Y} corresponds to line B-10 in Table B.

Table 71: Compounds I wherein L, r and R³ are as defined in Table 61; and wherein the combination of substituents R¹, Q and R^{V}/R^{Y} corresponds to line B-11 in Table B.

Table 72: Compounds I wherein L, r and R³ are as defined in Table 61; and wherein the combination of substituents R¹, Q and R^{V}/R^{Y} corresponds to line B-12 in Table B.

Table 73: Compounds I wherein L, r and R³ are as defined in Table 61; and wherein the combination of substituents R¹, Q and R^{V}/R^{Y} corresponds to line B-13 in Table B.

Table 74: Compounds I wherein L, r and R³ are as defined in Table 61; and wherein the combination of substituents R¹, Q and R^{V}/R^{Y} corresponds to line B-14 in Table B.

Table 75: Compounds I wherein L, r and R³ are as defined in Table 61; and wherein the combination of substituents R¹, Q and R^{V}/R^{Y} corresponds to line B-15 in Table B.

Table 76: Compounds I wherein L, r and R³ are as defined in Table 61; and wherein the combination of substituents R¹, Q and R^{V}/R^{Y} corresponds to line B-16 in Table B.

Table 77: Compounds I wherein L, r and R³ are as defined in Table 61; and wherein the combination of substituents R¹, Q and R^{V}/R^{Y} corresponds to line B-17 in Table B.

Table 78: Compounds I wherein L, r and R³ are as defined in Table 61; and wherein the combination of substituents R¹, Q and R^{V}/R^{Y} corresponds to line B-18 in Table B.

Table 79: Compounds I wherein L, r and R³ are as defined in Table 61; and wherein the combination of substituents R¹, Q and R^{V}/R^{Y} corresponds to line B-19 in Table B.

Table 80: Compounds I wherein L, r and R³ are as defined in Table 61; and wherein the combination of substituents R¹, Q and R^{V}/R^{Y} corresponds to line B-20 in Table B.

Table 81: Compounds I wherein L, r and R³ are as defined in Table 61; and wherein the combination of substituents R¹, Q and R^{V}/R^{Y} corresponds to line B-21 in Table B.

Table 82: Compounds I wherein L, r and R³ are as defined in Table 61; and wherein the combination of substituents R¹, Q and R^{V}/R^{Y} corresponds to line B-22 in Table B.

Table 83: Compounds I wherein L, r and R³ are as defined in Table 61; and wherein the combination of substituents R¹, Q and R^{V}/R^{Y} corresponds to line B-23 in Table B.

Table 84: Compounds I wherein L, r and R³ are as defined in Table 61; and wherein the combination of substituents R¹, Q and R^{V}/R^{Y} corresponds to line B-24 in Table B.

Table 85: Compounds I wherein L, r and R³ are as defined in Table 61; and wherein the combination of substituents R¹, Q and R^{V}/R^{Y} corresponds to line B-25 in Table B.

Table 86: Compounds I wherein L, r and R³ are as defined in Table 61; and wherein the combination of substituents R¹, Q and R^{V}/R^{Y} corresponds to line B-26 in Table B.

Table 87: Compounds I wherein L, r and R³ are as defined in Table 61; and wherein the combination of substituents R¹, Q and R^{V}/R^{Y} corresponds to line B-27 in Table B.

Table 88: Compounds I wherein L, r and R³ are as defined in Table 61; and wherein the combination of substituents R¹, Q and R^{V}/R^{Y} corresponds to line B-28 in Table B.

Table 89: Compounds I wherein L, r and R³ are as defined in Table 61; and wherein the combination of substituents R¹, Q and R^{V}/R^{Y} corresponds to line B-29 in Table B.

Table 90: Compounds I wherein L, r and R³ are as defined in Table 61; and wherein the combination of substituents R¹, Q and R^{V}/R^{Y} corresponds to line B-30 in Table B.

Table 91: Compounds I wherein L, r and R³ are as defined in Table 61; and wherein the combination of substituents R¹, Q and R^{V}/R^{Y} corresponds to line B-31 in Table B.

Table 92: Compounds I wherein L, r and R³ are as defined in Table 61; and wherein the combination of substituents R¹, Q and R^{V}/R^{Y} corresponds to line B-32 in Table B.

Table 93: Compounds I wherein L, r and R³ are as defined in Table 61; and wherein the combination of substituents R¹, Q and R^{V}/R^{Y} corresponds to line B-33 in Table B.

Table 94: Compounds I wherein L, r and R³ are as defined in Table 61; and wherein the combination of substituents R¹, Q and R^{V}/R^{Y} corresponds to line B-34 in Table B.

Table 95: Compounds I wherein L, r and R³ are as defined in Table 61; and wherein the combination of substituents R¹, Q and R^{V}/R^{Y} corresponds to line B-35 in Table B.

Table 96: Compounds I wherein L, r and R³ are as defined in Table 61; and wherein the combination of substituents R¹, Q and R^{V}/R^{Y} corresponds to line B-36 in Table B.

Table 97: Compounds I wherein L, r and R³ are as defined in Table 61; and wherein the combination of substituents R¹, Q and R^{V}/R^{Y} corresponds to line B-37 in Table B.

Table 98: Compounds I wherein L, r and R³ are as defined in Table 61; and wherein the combination of substituents R¹, Q and R^{V}/R^{Y} corresponds to line B-38 in Table B.

Table 99: Compounds I wherein L, r and R³ are as defined in Table 61; and wherein the combination of substituents R¹, Q and R^{V}/R^{Y} corresponds to line B-39 in Table B.

Table 100: Compounds I wherein L, r and R³ are as defined in Table 61; and wherein the combination of substituents R¹, Q and R^{V}/R^{Y} corresponds to line B-40 in Table B.

Table 101: Compounds I wherein L, r and R³ are as defined in Table 61; and wherein the combination of substituents R¹, Q and R^{V}/R^{Y} corresponds to line B-41 in Table B.

Table 102: Compounds I wherein L, r and R³ are as defined in Table 61; and wherein the combination of substituents R¹, Q and R^{V}/R^{Y} corresponds to line B-42 in Table B.

Table 103: Compounds I wherein L, r and R³ are as defined in Table 61; and wherein the combination of substituents R¹, Q and R^{V}/R^{Y} corresponds to line B-43 in Table B.

Table 104: Compounds I wherein L, r and R³ are as defined in Table 61; and wherein the combination of substituents R¹, Q and R^{V}/R^{Y} corresponds to line B-44 in Table B.

Table 105: Compounds I wherein L, r and R³ are as defined in Table 61; and wherein the combination of substituents R¹, Q and R^{V}/R^{Y} corresponds to line B-45 in Table B.

Table 106: Compounds I wherein L, r and R³ are as defined in Table 61; and wherein the combination of substituents R¹, Q and R^{V}/R^{Y} corresponds to line B-46 in Table B.

Table 107: Compounds I wherein L, r and R³ are as defined in Table 61; and wherein the combination of substituents R¹, Q and R^{V}/R^{Y} corresponds to line B-47 in Table B.

Table 108: Compounds I wherein L, r and R³ are as defined in Table 61; and wherein the combination of substituents R¹, Q and R^{V}/R^{Y} corresponds to line B-48 in Table B.

Table 109: Compounds I wherein L, r and R³ are as defined in Table 61; and wherein the combination of substituents R¹, Q and R^{V}/R^{Y} corresponds to line B-49 in Table B.

Table 110: Compounds I wherein L, r and R³ are as defined in Table 61; and wherein the combination of substituents R¹, Q and R^{V}/R^{Y} corresponds to line B-50 in Table B.

Table 111: Compounds I wherein L, r and R³ are as defined in Table 61; and wherein the combination of substituents R¹, Q and R^{V}/R^{Y} corresponds to line B-51 in Table B.

Table 112: Compounds I wherein L, r and R³ are as defined in Table 61; and wherein the combination of substituents R¹, Q and R^{V}/R^{Y} corresponds to line B-52 in Table B.

Table 113: Compounds I wherein L, r and R³ are as defined in Table 61; and wherein the combination of substituents R¹, Q and R^{V}/R^{Y} corresponds to line B-53 in Table B.

Table 114: Compounds I wherein L, r and R³ are as defined in Table 61; and wherein the combination of substituents R¹, Q and R^{V}/R^{Y} corresponds to line B-54 in Table B.

Table 115: Compounds I wherein L, r and R³ are as defined in Table 61; and wherein the combination of substituents R¹, Q and R^{V}/R^{Y} corresponds to line B-55 in Table B.

Table 116: Compounds I wherein L, r and R³ are as defined in Table 61; and wherein the combination of substituents R¹, Q and R^{V}/R^{Y} corresponds to line B-56 in Table B.

Table 117: Compounds I wherein L, r and R³ are as defined in Table 61; and wherein the combination of substituents R¹, Q and R^{V}/R^{Y} corresponds to line B-57 in Table B.

Table 118: Compounds I wherein L, r and R³ are as defined in Table 61; and wherein the combination of substituents R¹, Q and R^{V}/R^{Y} corresponds to line B-58 in Table B.

Table 119: Compounds I wherein L, r and R³ are as defined in Table 61; and wherein the combination of substituents R¹, Q and R^{V}/R^{Y} corresponds to line B-59 in Table B.

Table 120: Compounds I wherein L, r and R³ are as defined in Table 61; and wherein the combination of substituents R¹, Q and R^{V}/R^{Y} corresponds to line B-60 in Table B.

Table 121: Compounds I wherein L is —CH₂—O—N═C(CH₃)—, r is 0 and wherein the meaning of R³ is selected in each case from any one of the groups R3-1 to R3-225 in Table A; and wherein the combination of substituents R¹, Q and R^{V}/R^{Y} corresponds to line B-1 in Table B.

Table 122: Compounds I wherein L, r and R³ are as defined in Table 121; and wherein the combination of substituents R¹, Q and R^{V}/R^{Y} corresponds to line B-2 in Table B.

Table 123: Compounds I wherein L, r and R³ are as defined in Table 121; and wherein the combination of substituents R¹, Q and R^{V}/R^{Y} corresponds to line B-3 in Table B.

Table 124: Compounds I wherein L, r and R³ are as defined in Table 121; and wherein the combination of substituents R¹, Q and R^{V}/R^{Y} corresponds to line B-4 in Table B.

Table 125: Compounds I wherein L, r and R³ are as defined in Table 121; and wherein the combination of substituents R¹, Q and R^{V}/R^{Y} corresponds to line B-5 in Table B.

Table 126: Compounds I wherein L, r and R³ are as defined in Table 121; and wherein the combination of substituents R¹, Q and R^{V}/R^{Y} corresponds to line B-6 in Table B.

Table 127: Compounds I wherein L, r and R³ are as defined in Table 121; and wherein the combination of substituents R¹, Q and R^{V}/R^{Y} corresponds to line B-7 in Table B.

Table 128: Compounds I wherein L, r and R³ are as defined in Table 121; and wherein the combination of substituents R¹, Q and R^{V}/R^{Y} corresponds to line B-8 in Table B.

Table 129: Compounds I wherein L, r and R³ are as defined in Table 121; and wherein the combination of substituents R¹, Q and R^{V}/R^{Y} corresponds to line B-9 in Table B.

Table 130: Compounds I wherein L, r and R³ are as defined in Table 121; and wherein the combination of substituents R¹, Q and R^{V}/R^{Y} corresponds to line B-10 in Table B.

Table 131: Compounds I wherein L, r and R³ are as defined in Table 121; and wherein the combination of substituents R¹, Q and R^{V}/R^{Y} corresponds to line B-11 in Table B.

Table 132: Compounds I wherein L, r and R³ are as defined in Table 121; and wherein the combination of substituents R¹, Q and R^{V}/R^{Y} corresponds to line B-12 in Table B.

Table 133: Compounds I wherein L, r and R³ are as defined in Table 121; and wherein the combination of substituents R¹, Q and R^{V}/R^{Y} corresponds to line B-13 in Table B.

Table 134: Compounds I wherein L, r and R³ are as defined in Table 121; and wherein the combination of substituents R¹, Q and R^{V}/R^{Y} corresponds to line B-14 in Table B.

Table 135: Compounds I wherein L, r and R³ are as defined in Table 121; and wherein the combination of substituents R¹, Q and R^{V}/R^{Y} corresponds to line B-15 in Table B.

Table 136: Compounds I wherein L, r and R³ are as defined in Table 121; and wherein the combination of substituents R¹, Q and R^{V}/R^{Y} corresponds to line B-16 in Table B.

Table 137: Compounds I wherein L, r and R³ are as defined in Table 121; and wherein the combination of substituents R¹, Q and R^{V}/R^{Y} corresponds to line B-17 in Table B.

Table 138: Compounds I wherein L, r and R³ are as defined in Table 121; and wherein the combination of substituents R¹, Q and R^{V}/R^{Y} corresponds to line B-18 in Table B.

Table 139: Compounds I wherein L, r and R³ are as defined in Table 121; and wherein the combination of substituents R¹, Q and R^{V}/R^{Y} corresponds to line B-19 in Table B.

Table 140: Compounds I wherein L, r and R³ are as defined in Table 121; and wherein the combination of substituents R¹, Q and R^{V}/R^{Y} corresponds to line B-20 in Table B.

Table 141: Compounds I wherein L, r and R³ are as defined in Table 121; and wherein the combination of substituents R¹, Q and R^{V}/R^{Y} corresponds to line B-21 in Table B.

Table 142: Compounds I wherein L, r and R³ are as defined in Table 121; and wherein the combination of substituents R¹, Q and R^{V}/R^{Y} corresponds to line B-22 in Table B.

Table 143: Compounds I wherein L, r and R³ are as defined in Table 121; and wherein the combination of substituents R¹, Q and R^{V}/R^{Y} corresponds to line B-23 in Table B.

Table 144: Compounds I wherein L, r and R³ are as defined in Table 121; and wherein the combination of substituents R¹, Q and R^{V}/R^{Y} corresponds to line B-24 in Table B.

Table 145: Compounds I wherein L, r and R³ are as defined in Table 121; and wherein the combination of substituents R¹, Q and R^{V}/R^{Y} corresponds to line B-25 in Table B.

Table 146: Compounds I wherein L, r and R³ are as defined in Table 121; and wherein the combination of substituents R¹, Q and R^{V}/R^{Y} corresponds to line B-26 in Table B.

Table 147: Compounds I wherein L, r and R³ are as defined in Table 121; and wherein the combination of substituents R¹, Q and R^{V}/R^{Y} corresponds to line B-27 in Table B.

Table 148: Compounds I wherein L, r and R³ are as defined in Table 121; and wherein the combination of substituents R¹, Q and R^{V}/R^{Y} corresponds to line B-28 in Table B.

Table 149: Compounds I wherein L, r and R³ are as defined in Table 121; and wherein the combination of substituents R¹, Q and R^{V}/R^{Y} corresponds to line B-29 in Table B.

Table 150: Compounds I wherein L, r and R³ are as defined in Table 121; and wherein the combination of substituents R¹, Q and R^{V}/R^{Y} corresponds to line B-30 in Table B.

Table 151: Compounds I wherein L, r and R³ are as defined in Table 121; and wherein the combination of substituents R¹, Q and R^{V}/R^{Y} corresponds to line B-31 in Table B.

Table 152: Compounds I wherein L, r and R³ are as defined in Table 121; and wherein the combination of substituents R¹, Q and R^{V}/R^{Y} corresponds to line B-32 in Table B.

Table 153: Compounds I wherein L, r and R³ are as defined in Table 121; and wherein the combination of substituents R¹, Q and R^{V}/R^{Y} corresponds to line B-33 in Table B.

Table 154: Compounds I wherein L, r and R³ are as defined in Table 121; and wherein the combination of substituents R¹, Q and R^{V}/R^{Y} corresponds to line B-34 in Table B.

Table 155: Compounds I wherein L, r and R³ are as defined in Table 121; and wherein the combination of substituents R¹, Q and R^{V}/R^{Y} corresponds to line B-35 in Table B.

Table 156: Compounds I wherein L, r and R³ are as defined in Table 121; and wherein the combination of substituents R¹, Q and R^{V}/R^{Y} corresponds to line B-36 in Table B.

Table 157: Compounds I wherein L, r and R³ are as defined in Table 121; and wherein the combination of substituents R¹, Q and R^{V}/R^{Y} corresponds to line B-37 in Table B.

Table 158: Compounds I wherein L, r and R³ are as defined in Table 121; and wherein the combination of substituents R¹, Q and R^{V}/R^{Y} corresponds to line B-38 in Table B.

Table 159: Compounds I wherein L, r and R³ are as defined in Table 121; and wherein the combination of substituents R¹, Q and R^{V}/R^{Y} corresponds to line B-39 in Table B.

Table 160: Compounds I wherein L, r and R³ are as defined in Table 121; and wherein the combination of substituents R¹, Q and R^{V}/R^{Y} corresponds to line B-40 in Table B.

Table 161: Compounds I wherein L, r and R³ are as defined in Table 121; and wherein the combination of substituents R¹, Q and R^{V}/R^{Y} corresponds to line B-41 in Table B.

Table 162: Compounds I wherein L, r and R³ are as defined in Table 121; and wherein the combination of substituents R¹, Q and R^{V}/R^{Y} corresponds to line B-42 in Table B.

Table 163: Compounds I wherein L, r and R³ are as defined in Table 121; and wherein the combination of substituents R¹, Q and R^{V}/R^{Y} corresponds to line B-43 in Table B.

Table 164: Compounds I wherein L, r and R³ are as defined in Table 121; and wherein the combination of substituents R¹, Q and R^{V}/R^{Y} corresponds to line B-44 in Table B.

Table 165: Compounds I wherein L, r and R³ are as defined in Table 121; and wherein the combination of substituents R¹, Q and R^{V}/R^{Y} corresponds to line B-45 in Table B.

Table 166: Compounds I wherein L, r and R³ are as defined in Table 121; and wherein the combination of substituents R¹, Q and R^{V}/R^{Y} corresponds to line B-46 in Table B.

Table 167: Compounds I wherein L, r and R³ are as defined in Table 121; and wherein the combination of substituents R¹, Q and R^{V}/R^{Y} corresponds to line B-47 in Table B.

Table 168: Compounds I wherein L, r and R³ are as defined in Table 121; and wherein the combination of substituents R¹, Q and R^{V}/R^{Y} corresponds to line B-48 in Table B.

Table 169: Compounds I wherein L, r and R³ are as defined in Table 121; and wherein the combination of substituents R¹, Q and R^{V}/R^{Y} corresponds to line B-49 in Table B.

Table 170: Compounds I wherein L, r and R³ are as defined in Table 121; and wherein the combination of substituents R¹, Q and R^{V}/R^{Y} corresponds to line B-50 in Table B.

Table 171: Compounds I wherein L, r and R³ are as defined in Table 121; and wherein the combination of substituents R¹, Q and R^{V}/R^{Y} corresponds to line B-51 in Table B.

Table 172: Compounds I wherein L, r and R³ are as defined in Table 121; and wherein the combination of substituents R¹, Q and R^{V}/R^{Y} corresponds to line B-52 in Table B.

Table 173: Compounds I wherein L, r and R³ are as defined in Table 121; and wherein the combination of substituents R¹, Q and R^{V}/R^{Y} corresponds to line B-53 in Table B.

Table 174: Compounds I wherein L, r and R³ are as defined in Table 121; and wherein the combination of substituents R¹, Q and R^{V}/R^{Y} corresponds to line B-54 in Table B.

Table 175: Compounds I wherein L, r and R³ are as defined in Table 121; and wherein the combination of substituents R¹, Q and R^{V}/R^{Y} corresponds to line B-55 in Table B.

Table 176: Compounds I wherein L, r and R³ are as defined in Table 121; and wherein the combination of substituents R¹, Q and R^{V}/R^{Y} corresponds to line B-56 in Table B.

Table 177: Compounds I wherein L, r and R³ are as defined in Table 121; and wherein the combination of substituents R¹, Q and R^{V}/R^{Y} corresponds to line B-57 in Table B.

Table 178: Compounds I wherein L, r and R³ are as defined in Table 121; and wherein the combination of substituents R¹, Q and R^{V}/R^{Y} corresponds to line B-58 in Table B.

Table 179: Compounds I wherein L, r and R³ are as defined in Table 121; and wherein the combination of substituents R¹, Q and R^{V}/R^{Y} corresponds to line B-59 in Table B.

Table 180: Compounds I wherein L, r and R³ are as defined in Table 121; and wherein the combination of substituents R¹, Q and R^{V}/R^{Y} corresponds to line B-60 in Table B.

Table 181: Compounds I wherein L is —O—N═C(CH₃)—, r is 0 and wherein the meaning of R³ is selected in each case from any one of the groups R3-1 to R3-225 in Table A; and wherein the combination of substituents R¹, Q and R^{V}/R^{Y} corresponds to line B-1 in Table B.

Table 182: Compounds I wherein L, r and R³ are as defined in Table 181; and wherein the combination of substituents R¹, Q and R^{V}/R^{Y} corresponds to line B-2 in Table B.

Table 183: Compounds I wherein L, r and R³ are as defined in Table 181; and wherein the combination of substituents R¹, Q and R^{V}/R^{Y} corresponds to line B-3 in Table B.

Table 184: Compounds I wherein L, r and R³ are as defined in Table 181; and wherein the combination of substituents R¹, Q and R^{V}/R^{Y} corresponds to line B-4 in Table B.

Table 185: Compounds I wherein L, r and R³ are as defined in Table 181; and wherein the combination of substituents R¹, Q and R^{V}/R^{Y} corresponds to line B-5 in Table B.

Table 186: Compounds I wherein L, r and R³ are as defined in Table 181; and wherein the combination of substituents R¹, Q and R^{V}/R^{Y} corresponds to line B-6 in Table B.

Table 187: Compounds I wherein L, r and R³ are as defined in Table 181; and wherein the combination of substituents R¹, Q and R^{V}/R^{Y} corresponds to line B-7 in Table B.

Table 188: Compounds I wherein L, r and R³ are as defined in Table 181; and wherein the combination of substituents R¹, Q and R^{V}/R^{Y} corresponds to line B-8 in Table B.

Table 189: Compounds I wherein L, r and R³ are as defined in Table 181; and wherein the combination of substituents R¹, Q and R^{V}/R^{Y} corresponds to line B-9 in Table B.

Table 190: Compounds I wherein L, r and R³ are as defined in Table 181; and wherein the combination of substituents R¹, Q and R^{V}/R^{Y} corresponds to line B-10 in Table B.

Table 191: Compounds I wherein L, r and R³ are as defined in Table 181; and wherein the combination of substituents R¹, Q and R^{V}/R^{Y} corresponds to line B-11 in Table B.

Table 192: Compounds I wherein L, r and R³ are as defined in Table 181; and wherein the combination of substituents R¹, Q and R^{V}/R^{Y} corresponds to line B-12 in Table B.

Table 193: Compounds I wherein L, r and R³ are as defined in Table 181; and wherein the combination of substituents R¹, Q and R^{V}/R^{Y} corresponds to line B-13 in Table B.

Table 194: Compounds I wherein L, r and R³ are as defined in Table 181; and wherein the combination of substituents R¹, Q and R^{V}/R^{Y} corresponds to line B-14 in Table B.

Table 195: Compounds I wherein L, r and R³ are as defined in Table 181; and wherein the combination of substituents R¹, Q and R^{V}/R^{Y} corresponds to line B-15 in Table B.

Table 196: Compounds I wherein L, r and R³ are as defined in Table 181; and wherein the combination of substituents R¹, Q and R^{V}/R^{Y} corresponds to line B-16 in Table B.

Table 197: Compounds I wherein L, r and R³ are as defined in Table 181; and wherein the combination of substituents R¹, Q and R^{V}/R^{Y} corresponds to line B-17 in Table B.

Table 198: Compounds I wherein L, r and R³ are as defined in Table 181; and wherein the combination of substituents R¹, Q and R^{V}/R^{Y} corresponds to line B-18 in Table B.

Table 199: Compounds I wherein L, r and R³ are as defined in Table 181; and wherein the combination of substituents R¹, Q and R^{V}/R^{Y} corresponds to line B-19 in Table B.

Table 200: Compounds I wherein L, r and R³ are as defined in Table 181; and wherein the combination of substituents R¹, Q and R^{V}/R^{Y} corresponds to line B-20 in Table B.

Table 201: Compounds I wherein L, r and R³ are as defined in Table 181; and wherein the combination of substituents R¹, Q and R^{V}/R^{Y} corresponds to line B-21 in Table B.

Table 202: Compounds I wherein L, r and R³ are as defined in Table 181; and wherein the combination of substituents R¹, Q and R^{V}/R^{Y} corresponds to line B-22 in Table B.

Table 203: Compounds I wherein L, r and R³ are as defined in Table 181; and wherein the combination of substituents R¹, Q and R^{V}/R^{Y} corresponds to line B-23 in Table B.

Table 204: Compounds I wherein L, r and R³ are as defined in Table 181; and wherein the combination of substituents R¹, Q and R^{V}/R^{Y} corresponds to line B-24 in Table B.

Table 205: Compounds I wherein L, r and R³ are as defined in Table 181; and wherein the combination of substituents R¹, Q and R^{V}/R^{Y} corresponds to line B-25 in Table B.

Table 206: Compounds I wherein L, r and R³ are as defined in Table 181; and wherein the combination of substituents R¹, Q and R^{V}/R^{Y} corresponds to line B-26 in Table B.

Table 207: Compounds I wherein L, r and R³ are as defined in Table 181; and wherein the combination of substituents R¹, Q and R^{V}/R^{Y} corresponds to line B-27 in Table B.

Table 208: Compounds I wherein L, r and R³ are as defined in Table 181; and wherein the combination of substituents R¹, Q and R^{V}/R^{Y} corresponds to line B-28 in Table B.

Table 209: Compounds I wherein L, r and R³ are as defined in Table 181; and wherein the combination of substituents R¹, Q and R^{V}/R^{Y} corresponds to line B-29 in Table B.

Table 210: Compounds I wherein L, r and R³ are as defined in Table 181; and wherein the combination of substituents R¹, Q and R^{V}/R^{Y} corresponds to line B-30 in Table B.

Table 211: Compounds I wherein L, r and R³ are as defined in Table 181; and wherein the combination of substituents R¹, Q and R^{V}/R^{Y} corresponds to line B-31 in Table B.

Table 212: Compounds I wherein L, r and R³ are as defined in Table 181; and wherein the combination of substituents R¹, Q and R^{V}/R^{Y} corresponds to line B-32 in Table B.

Table 213: Compounds I wherein L, r and R³ are as defined in Table 181; and wherein the combination of substituents R¹, Q and R^{V}/R^{Y} corresponds to line B-33 in Table B.

Table 214: Compounds I wherein L, r and R³ are as defined in Table 181; and wherein the combination of substituents R¹, Q and R^{V}/R^{Y} corresponds to line B-34 in Table B.

Table 215: Compounds I wherein L, r and R³ are as defined in Table 181; and wherein the combination of substituents R¹, Q and R^{V}/R^{Y} corresponds to line B-35 in Table B.

Table 216: Compounds I wherein L, r and R³ are as defined in Table 181; and wherein the combination of substituents R¹, Q and R^{V}/R^{Y} corresponds to line B-36 in Table B.

Table 217: Compounds I wherein L, r and R³ are as defined in Table 181; and wherein the combination of substituents R¹, Q and R^{V}/R^{Y} corresponds to line B-37 in Table B.

Table 218: Compounds I wherein L, r and R³ are as defined in Table 181; and wherein the combination of substituents R¹, Q and R^{V}/R^{Y} corresponds to line B-38 in Table B.

Table 219: Compounds I wherein L, r and R³ are as defined in Table 181; and wherein the combination of substituents R¹, Q and R^{V}/R^{Y} corresponds to line B-39 in Table B.

Table 220: Compounds I wherein L, r and R³ are as defined in Table 181; and wherein the combination of substituents R¹, Q and R^{V}/R^{Y} corresponds to line B-40 in Table B.

Table 221: Compounds I wherein L, r and R³ are as defined in Table 181; and wherein the combination of substituents R¹, Q and R^{V}/R^{Y} corresponds to line B-41 in Table B.

Table 222: Compounds I wherein L, r and R³ are as defined in Table 181; and wherein the combination of substituents R¹, Q and R^{V}/R^{Y} corresponds to line B-42 in Table B.

Table 223: Compounds I wherein L, r and R³ are as defined in Table 181; and wherein the combination of substituents R¹, Q and R^{V}/R^{Y} corresponds to line B-43 in Table B.

Table 224: Compounds I wherein L, r and R³ are as defined in Table 181; and wherein the combination of substituents R¹, Q and R^{V}/R^{Y} corresponds to line B-44 in Table B.

Table 225: Compounds I wherein L, r and R³ are as defined in Table 181; and wherein the combination of substituents R¹, Q and R^{V}/R^{Y} corresponds to line B-45 in Table B.

Table 226: Compounds I wherein L, r and R³ are as defined in Table 181; and wherein the combination of substituents R¹, Q and R^{V}/R^{Y} corresponds to line B-46 in Table B.

Table 227: Compounds I wherein L, r and R³ are as defined in Table 181; and wherein the combination of substituents R¹, Q and R^{V}/R^{Y} corresponds to line B-47 in Table B.

Table 228: Compounds I wherein L, r and R³ are as defined in Table 181; and wherein the combination of substituents R¹, Q and R^{V}/R^{Y} corresponds to line B-48 in Table B.

Table 229: Compounds I wherein L, r and R³ are as defined in Table 181; and wherein the combination of substituents R¹, Q and R^{V}/R^{Y} corresponds to line B-49 in Table B.

Table 230: Compounds I wherein L, r and R³ are as defined in Table 181; and wherein the combination of substituents R¹, Q and R^{V}/R^{Y} corresponds to line B-50 in Table B.

Table 231: Compounds I wherein L, r and R³ are as defined in Table 181; and wherein the combination of substituents R¹, Q and R^{V}/R^{Y} corresponds to line B-51 in Table B.

Table 232: Compounds I wherein L, r and R³ are as defined in Table 181; and wherein the combination of substituents R¹, Q and R^{V}/R^{Y} corresponds to line B-52 in Table B.

Table 233: Compounds I wherein L, r and R³ are as defined in Table 181; and wherein the combination of substituents R¹, Q and R^{V}/R^{Y} corresponds to line B-53 in Table B.

Table 234: Compounds I wherein L, r and R³ are as defined in Table 181; and wherein the combination of substituents R¹, Q and R^{V}/R^{Y} corresponds to line B-54 in Table B.

Table 235: Compounds I wherein L, r and R³ are as defined in Table 181; and wherein the combination of substituents R¹, Q and R^{V}/R^{Y} corresponds to line B-55 in Table B.

Table 236: Compounds I wherein L, r and R³ are as defined in Table 181; and wherein the combination of substituents R¹, Q and R^{V}/R^{Y} corresponds to line B-56 in Table B.

Table 237: Compounds I wherein L, r and R³ are as defined in Table 181; and wherein the combination of substituents R¹, Q and R^{V}/R^{Y} corresponds to line B-57 in Table B.

Table 238: Compounds I wherein L, r and R³ are as defined in Table 181; and wherein the combination of substituents R¹, Q and R^{V}/R^{Y} corresponds to line B-58 in Table B.

Table 239: Compounds I wherein L, r and R³ are as defined in Table 181; and wherein the combination of substituents R¹, Q and R^{V}/R^{Y} corresponds to line B-59 in Table B.

Table 240: Compounds I wherein L, r and R³ are as defined in Table 181; and wherein the combination of substituents R¹, Q and R^{V}/R^{Y} corresponds to line B-60 in Table B.

The present invention furthermore relates to processes for preparing compounds I. Compounds I can be prepared starting from commercially available halogenated benzene derivatives as described as follows:

Compounds I, wherein Q is —NH— can be prepared starting from anilines 1, which are commercial available or easily accessible through procedures that are known to the skilled person, which can then be converted to hydrazine 2 according to Scheme 1. Treatment with halogenated chloro formiates or carbamates 3a, wherein p in dicates the number of substituents R^{X} and p is 1, 2 or 3 and wherein indicates the number of carbon units and n is 0, 1, 2, 3 or 4, lead to the formation of N-amino carbamates (Y═NH) or analogous carbonates (Y═O), i.e. compounds 3, as described in DE3033161 A1. Appropriate choice of starting materials enables the preparation of compounds wherein Q is —(NQ^{a})- and/or Y is —(NY^{a})—.

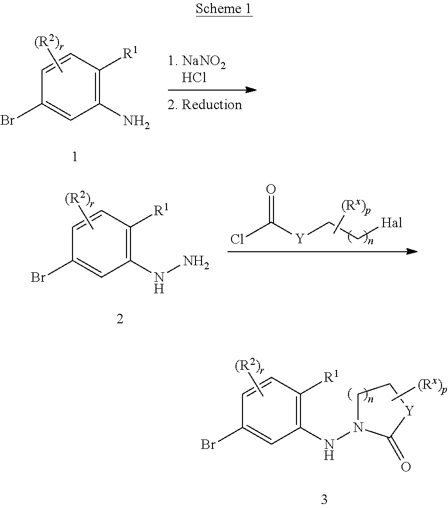

Alternatively, compounds 3 can be prepared through a palladium-catalyzed cross coupling of the corresponding aryl bromide with N-amino carbamates as described in Synlett 2011, 17, 2555-2558.

Compounds I, wherein Q is —(CQ^{b}Q^{c})- can be synthesized from benzylic bromide 4 as shown, for example, in Scheme 2, which is either commercially available or easily accessible from commercially available toluene derivatives through radical bromination, as described, for example, in WO 09/100170 A1. Treatment of compounds 4 with cyclic carbamates or ureas in the presence of a base, for example NaH, yields benzylic carbamates or ureas of the formula 5 (see for example Bioorg. Med. Chem. 2010, 18, 4570-4590 or Synthesis 2009, 11, 1897-1903). The synthesis of compounds 5 with further heteroatoms incorporated into the cyclic carbamate or urea can be accomplished in an analogous fashion by employment of the corresponding saturated heterocycles as described, for example, in J. Org. Chem. 1995, 60, 6946-6952 or Synlett 1997, 3, 263-264 or J. Org. Chem. 2002, 67, 8871-8876.

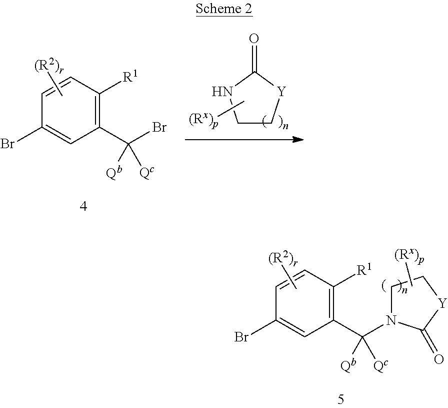

The installation of the groups R³-L- in compounds of the formula 3 or 5, wherein L is —CH₂—, can be achieved through metal-catalyzed cross couplings of aryl bromides of the formula II, wherein X is a leaving group, such as Cl, Br, iodine, alkylsulfonate, haloalkylsulfonate or phenylsulfonate, wherein the phenyl ring in the last mentioned group is unsubstituted or substituted by 1, 2 or 3 identical or different substituents selected from halogen, cyano, nitro, C₁-C₆-alkyl or C₁-C₆-haloalkyl; preferably X is Cl or Br, for example compounds 3 or 5, with organometall compounds to produce target compounds 6 as shown in Scheme 3. A wide range of different organometallic compounds and catalysts can be employed, such as nickel- as well as palladium-catalysts in combination with organo-zinc, -magnesium or -tin compounds. Representative examples for such conversions can be found in J. Org. Chem. 1977, 42, 1821-1823; J. Org. Chem. 2008, 73, 8422-8436; Catalysis Letters 2012, 142, 557-565 and Eur. J. Inorg. Chem. 2012, 8, 1269-1277.

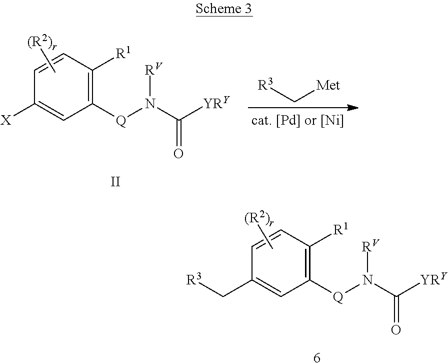

In a similar way compounds 7 in Scheme 4, wherein L is —CH₂CH₂—, can be obtained through palladium catalysis with the corresponding alkyl-zinc and -indium compounds or with alkyl boronic acids as described for example in Tetrahedron 2002, 58, 1465-1470; J. Org. Chem. 2003, 68, 5534-5539; Org. Lett. 2007, 9, 4571-4574 and Angew. Chem. Int. Ed. 2003, 68, 5534-5539.

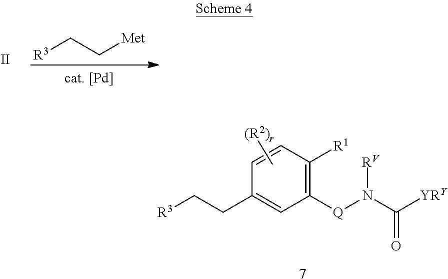

Benzylic alcohol 8 as shown in Scheme 5 is a precursor for the preparation of compounds I, wherein L is —OCH₂—. Compounds 8 can be prepared using organotin compounds II.a in a Stille-coupling as described in Chemistry Letters 1985, 7, 997-998 or WO 05/110992 A1 and depicted in Scheme 5.

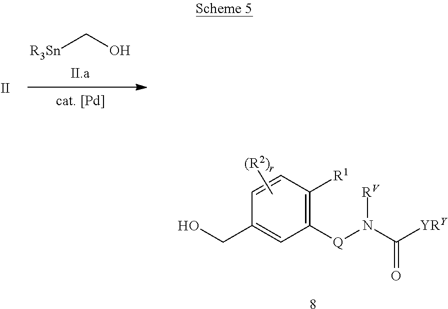

Alternatively, a halogen-metal exchange and subsequent trapping of the arylanion with formaldehyde or with N,N-dimethyl formamide, followed by reduction of thus obtained aldehyde also leads to the formation of compound 8 (see for example: Tetrahedron 2008, 64, 11449-11461; EP 2161320 A2 or J. Chem. Soc., Perkin 1, 1987, 1573-1578).

Target compounds I, wherein L is —OCH₂— (compound 9) and wherein W is O, can be prepared from compounds of the formula III by reaction with compounds III.a in analogy to known methods as described, for example, in WO 12/133607 A1 and as shown in Scheme 6. The group T in compounds III is a leaving group, such as OH, Cl, Br, iodine, alkylsulfonate, haloalkylsulfonate or phenylsulfonate, wherein the phenyl ring in the last mentioned group is unsubstituted or substituted by 1, 2 or 3 identical or different substituents selected from halogen, cyano, nitro, C₁-C₆-alkyl or C₁-C₆-haloalkyl; preferably T is Cl or Br.

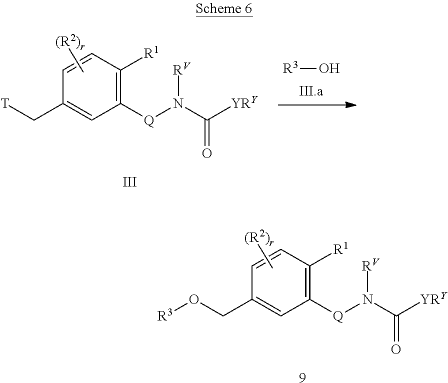

Compounds of the formula 11, wherein L is selected from one of the groups —C(Z)═N—O—CH₂—, —CHZ—C(Z)═N—O—CH₂—, —O—N═C(Z)—C(Z)═N—O—CH₂—, —C(═O)—C(Z)═N—O—CH₂— and —C(═N—O—Z)—C(Z)═N—O—CH₂—, can be prepared starting from compounds of the formula III as outlined in Scheme 7. Hydroxylamine 10 can be synthesized by a substitution reaction of compound III for example with N-hydroxyphthalimide and subsequent cleavage of the phthalimide residue as described in J. Org. Chem. 2005, 70, 6991-6994 or Bioorg. Med. Chem. Lett. 2003, 13, 3155-3159. Subsequent condensation of compound 10 with suitable aldehydes or ketones R³—C(═O)—Z, wherein Z has the meaning as defined for compounds I, leads to the corresponding oxime ethers 11.

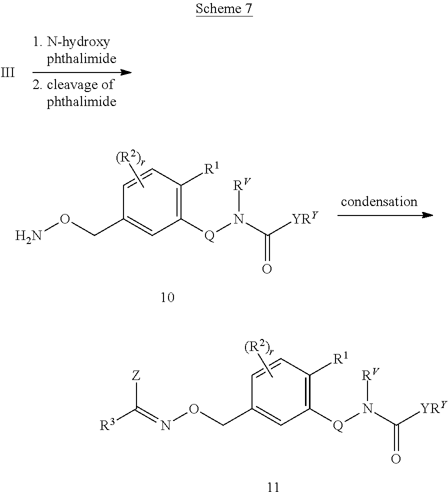

Alternatively, compounds 11 are also accessible through reaction of compounds III with oximes III.b as illustrated in Scheme 8 and as described in Synthesis 2010, 10, 1724-1730.

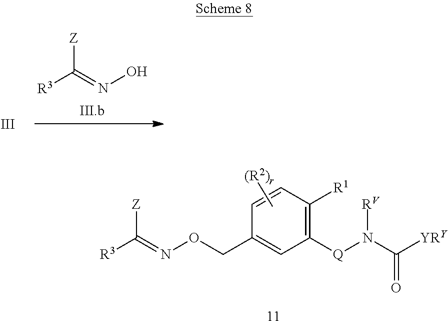

Compounds III.a and III.b and their synthesis is either known in the art or can be accomplished following standard procedures as described in the art.

To access compounds I, wherein L is —CH₂—O—N═C(Z)— or —O—N═C(Z)—, a palladium catalyzed Stille coupling of compounds II with alkoxyvinyltin reagents can be employed as described in Synthesis 2001, 10, 1551-1555 or Bioorg. Med. Chem. Lett. 2002, 12, 2043-2046 and as depicted in Scheme 9. Subsequent condensation of the resulting intermediate carbonyl compound with suitable hydroxylamines yields oxime ethers 12 as shown in Scheme 9. As an alternative a palladium catalyzed reaction of compounds II with acetic anhydride can also be employed (for example: Org. Lett. 2003, 5, 289-291; WO 08/124092 A2 or WO 11/059619 A1).

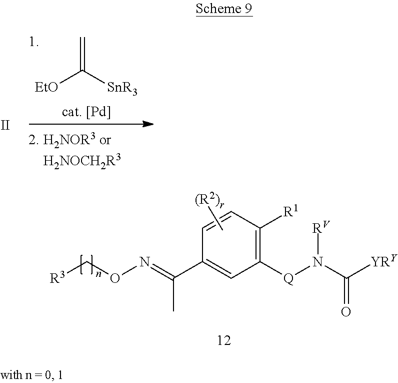

Compounds 13 in Scheme 10, wherein L is —O—, can be obtained through a copper or palladium catalyzed coupling of compounds II and alcohol III.a (Scheme 10) as described in Org. Lett. 2007, 9, 643-646; Org. Lett. 2012, 14, 170-173; J. Med. Chem. 2010, 53, 8679-8687 or US 2011/0237636 A1.

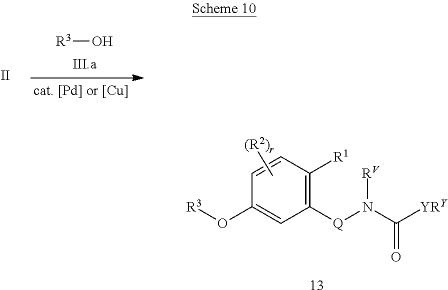

For the synthesis of bialy's 14 in Scheme 11, wherein L is a direct bond, a variety of different methods such as those described in WO 08/124092 A2 or WO 11/059619 A1 can be employed depending on the nature of the aromatic ring of R³.

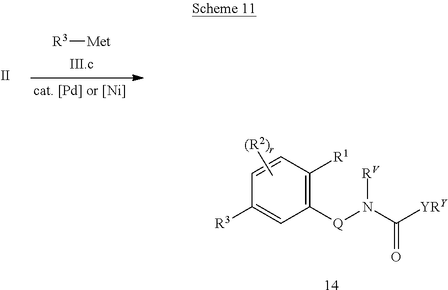

Depending on the nature of the starting materials it may be advantageous to prepare compounds I in a reversed order of transformations as compared to the syntheses described in Schemes 3 to 11, in the sense that benzylic bromide 4 may be subjected to a metal-catalyzed cross coupling in a first step to install the side chain followed by the installation of the carbamate according to the procedures described herein.

Compounds I, wherein W is S, can be prepared from the corresponding oxo analogues, i.e. wherein W is O, for example in analogy to methods described in US 20100022538 A1, J. Med. Chem. (2011), 54(9), 3241-3250, J. Org. Chem. (2011), 76(6), 1546-1553, Org. Lett. (2010) or 12(23), 5570-5572.

Compounds I, wherein Q is —C(═N—O-Qa)—, can be prepared from the corresponding oxo analogues, i.e. wherein Q is —C(═O)—, in analogy to WO 2007/075598 or from compounds I wherein Q is —C(═S)— according to WO 2008/039520 and O'zbekiston Kimyo Jurnali (2004) 4, 3-6.

Preference is also given to the uses, methods, mixtures and compositions, wherein the definitions (such as phytopathogenic fungi, treatments, crops, compounds II, further active ingredients, solvents, solid carriers) have independently of each other or more preferably in combination the following meanings and even more preferably in combination (any possible combination of 2 or more definitions as provided herein) with the preferred meanings of compounds I herein:

According to one embodiment of the invention, the invention also relates to a method for combating phytopathogenic fungi containing a mutation in the mitochondrial cytochrome b gene conferring resistance to Qo inhibitors, comprising: treating the phytopathogenic fungi containing a mutation in the mitochondrial cytochrome b gene conferring resistance to Qo inhibitors or the materials, plants, the soil or seeds that are at risk of being diseased from phytopathogenic fungi containing a mutation in the mitochondrial cytochrome b gene conferring resistance to Qo inhibitors with an effective amount of at least one compound I, or a composition comprising it thereof.

The term “phytopathogenic fungi containing a mutation in the mitochondrial cytochrome b gene conferring resistance to Qo inhibitors” ist be understood that at least 10% of the fungal isolates to be controlled contain a mutation in the mitochondrial cytochrome b gene conferring resistance to Qo inhibitors, more preferably at least 30%, even more preferably at least 50%, and most preferably at least 75% of the fungi, in particular between 90 and 100%.

It has been observed under field conditions that populations of phytopathogenic fungi apparently consisting of non-resistant strains can readily develop resistance. The compounds can be applied under such conditions, too, in order to prevent the formation of resistance and the spread of resistant strains altogether. In this regard it is useful that they have strong activity against non-resistant phytopathogenic fungi also.

According to another embodiment, the method for combating phytopathogenic fungi, comprises: a) identifying the phytopathogenic fungi containing a mutation in the mitochondrial cytochrome b gene conferring resistance to Qo inhibitors, or the materials, plants, the soil or seeds that are at risk of being diseased from phytopathogenic fungi as defined herein, and b) treating said fungi or the materials, plants, the soil or seeds with an effective amount of at least one compound I, or a composition comprising it thereof.

According to another embodiment of the invention, the invention also relates to a method for combating phytopathogenic fungi containing a mutation in the mitochondrial cytochrome b gene conferring resistance to Qo inhibitors, comprising: treating the phytopathogenic fungi whereof at least 10% contain a mutation in the mitochondrial cytochrome b gene conferring resistance to Qo inhibitors or the materials, plants, the soil or seeds that are at risk of being diseased from phytopathogenic fungi containing a mutation in the mitochondrial cytochrome b gene conferring resistance to Qo inhibitors with an effective amount of at least one compound I, or a composition comprising it thereof; more preferably at least 30%, even more preferably at least 50%, and most preferably at least 75% of the fungi contain a mutation in the mitochondrial cytochrome b gene conferring resistance to Qo inhibitors.

According to one embodiment of the use and the method for combating phytopathogenic fungi, wherein the mutation in the mitochondrial cytochrome b gene of the phytopathogenic fungi is G143A.

According to another embodiment, the phytopathogenic fungi are selected from the group consisting of basidomycetes, ascomycetes, and oomycetes.

According to a further embodiment, the phytopathogenic fungi are selected from the group consisting of Alternaria alternata, Blumeria graminis, Pyriculania oryzae (also known as Magnaporthe grisea), Septoria tritici (also known as Mycosphaerella graminicola), Mycosphaerella fijiensis, Venturia inaequalis, Pyrenophora teres, Pyrenophona tritici-repentis and Plasmopara viticola, in particular Septoria tritici.

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants. Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties. Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme phosphinothricin-N-acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the Mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici(anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola:Anthracnose stalk rot), soft fruits, potatoes (e. g. C. coccodes black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemilieia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or, rotbrenner', anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni(Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsil or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria]nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes. The compounds I and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubennann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof. Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof. Suitable surfactants are surface-active compounds, such as anionic, cationic, nonionic and amphoteric surfactants, block polymers, polyelectrolytes, and mixtures thereof. Such surfactants can be used as emulisifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I and 5-15 wt % wetting agent (e. g. alcohol alkoxylates) are dissolved in water and/or in a water-soluble solvent (e. g. alcohols) ad 100 wt %. The active substance dissolves upon dilution with water.

### ii) Dispersible Concentrates (DC)

5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

### iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

### vi) Water-Dispersible Granules and Water-Soluble Granules (WG, SG)

50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

### ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

### x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

### xi) Dustable Powders (DP, DS)

1-10 wt % of a compound I are ground finely and mixed intimately with solid carrier (e. g. finely divided kaolin) ad 100 wt %.

xii) Granules (GR, FG)

0.5-30 wt % of a compound I is ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

Biopesticides have been defined as a form of pesticides based on micro-organisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:
(1) Microbial pesticides consist of bacteria, fungi or viruses (and often include the metabolites that bacteria and fungi produce). Entomopathogenic nematodes are also classed as microbial pesticides, even though they are multi-cellular.
(2) Biochemical pesticides are naturally occurring substances that control pests or provide other crop protection uses as defined below, but are relatively non-toxic to mammals.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

When living microorganisms, such as microbial pesticides from groups L1), L3) and L5), form part of such kit, it must be taken care that choice and amounts of the components (e. g. chemical pesticides) and of the further auxiliaries should not influence the viability of the microbial pesticides in the composition mixed by the user. Especially for bactericides and solvents, compatibility with the respective microbial pesticide has to be taken into account.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e. g. pesticidally-active substances and biopesticides), in conjunction with which the compounds I can be used, is intended to illustrate the possible combinations but does not limit them:

### A) Respiration Inhibitors

Inhibitors of complex III at Qₒ site (e. g. strobilurins): azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneanninooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), 1-[3-chloro-2-[[[1-(4-chlorophenyl)-1H-pyrazol-3-yl]oxy]methyl]-phenyl]-1,4-dihydro-4-methyl-5H-tetrazol-5-one (A.1.23), (2E,3Z)-5-[[1-(2,4-dichlorophenyl)-1H-pyrazol-3-yl]oxy]-2-(methoxyimino)-N,3-dimethyl-pent-3-enamide (A.1.24), (2E,3Z)-5-[[1-(4-chlorophenyl)-1H-pyrazol-3-yl]oxyl]-2-(methoxyimino)-N,3-dimethyl-pent-3-enamide (A.1.25); (Z,2E)-5-[1-(4-chloro-2-fluoro-phenyl)pyrazol-3-yl]oxy-2-methoxyimino-N,3-dimethyl-pent-3-enamide (A.1.26)
inhibitors of complex III at Q site: cyazofamid (A.2.1), amisulbrom (A.2.2), [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate (A.2.3), [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate (A.2.4), [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate (A.2.5), [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate (A.2.7);
inhibitors of complex II (e. g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoromethylthiobiphenyl-2-yl)-3-difluoromethyl-1-methyl-1H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoromethyl)-1-methyl-N-(1,1,3-trimethylindan-4-yl)pyrazole-4-carboxamide (A.3.22), 1,3-dimethyl-N-(1,1,3-trimethylindan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);
other respiration inhibitors (e. g. complex I, uncouplers): diflumetorim (A.4.1), (5,8-difluoro-quinazolin-4-yl)-{2-[2-fluoro-4-(4-trifluoromethylpyridin-2-yloxy)-phenyl]-ethyl}amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

### B) Sterol Biosynthesis Inhibitors (SBI Fungicides)

C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S;3R)-3-(2-chlorophenyl)-2-(2,4-difluorophenyl)-oxiranylmethyl]-5-thiocyanato-1H-[1,2,4]triazolo (B.1.31), 2-[rel-(2S,3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chlorophenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.51); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol-4-yl]-(3-pyridyl)methanol (B.1.50);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);
others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

### D) Inhibitors of Cell Division and Cytoskeleton

tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);
other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

### E) Inhibitors of Amino Acid and Protein Synthesis

methionine synthesis inhibitors (anilino-pyrimidines): cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

### F) Signal Transduction Inhibitors

MAP/histidine kinase inhibitors: fluoroimid (F.1.1), iprodione (F.1.2), procymidone (F.1.3), vinclozolin (F.1.4), fenpiclonil (F.1.5), fludioxonil (F.1.6);
G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenyl)ethanesulfonyl)-but-2-yl) carbamic acid-(4-fluorophenyl) ester (G.3.8);
compounds affecting cell membrane permeability and fatty acides: propamocarb (G.4.1);
fatty acid amide hydrolase inhibitors: oxathiapiprolin (G.5.1), 2-{3-[2-(1-{[3,5-bis(di-fluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3);
   H) Inhibitors with Multi Site Action
inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds (e. g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11), N-(4-chloro-2-nitro-phenyl)-N-ethyl-4-methyl-benzenesulfonamide (H.3.12);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

inhibitors of glucan synthesis: validamycin (I.1.1), polyoxin B (I.1.2);
melanin synthesis inhibitors: pyroquilon (I.2.1), tricyclazole (I.2.2), carpropamid (I.2.3), dicyclomet (I.2.4), fenoxanil (I.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclomezine (K.1.7), difenzoquat (K.1.8), difenzoquat-methylsulfate (K.1.9), diphenylamin (K.1.10), fenpyrazamine (K.1.11), flumetover (K.1.12), flusulfamide (K.1.13), flutianil (K.1.14), methasulfocarb (K.1.15), nitrapyrin (K.1.16), nitrothal-isopropyl (K.1.18), oxathiapiprolin (K.1.19), tolprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K.1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K.1.27), 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone (K.1.28), 2-[3,5-bis(difluoromethyl)-1H-pyrazol-1-yl]-1-[4-(4-{5-[2-chloro-6-(prop-2-yn-1-yl-oxy)phenyl]-4,5-dihydro-1,2-oxazol-3-yl}-1,3-thiazol-2-yl)piperidin-1-yl]ethanone (K.1.29), N-(cyclopropylmethoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-trimethyl-silanyl-propoxy)-phenyl)-N-ethyl-N-methyl formamidine (K.1.33), N′-(5-difluoromethyl-2-methyl-4-(3-trimethylsilanyl-propoxy)-phenyl)-N-ethyl-N-methyl formamidine (K.1.34), methoxy-acetic acid 6-tert-butyl-8-fluoro-2,3-dimethyl-quinolin-4-yl ester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dimethoxy-pyrimidin-2-yl)-2-methyl-1H-benzoimidazole (K.1.39), 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluoro-phenyl]propan-2-ol (K.1.43), 2-[2-fluoro-6-[(8-fluoro-2-methyl-3-quinolyl)oxy]phen-yl]propan-2-ol (K.1.44), 3-(5-fluoro-3,3,4,4-tetramethyl-3,4-dihydroisoquinolin-1-yl)quinoline (K.1.45), 3-(4,4-difluoro-3,3-dimethyl-3,4-dihydroisoquinolin-1-yl)quinoline (K.1.46), 3-(4,4,5-trifluoro-3,3-dimethyl-3,4-dihydroisoquinolin-1-yl)quinoline (K.1.47), 9-fluoro-2,2-dimethyl-5-(3-quinolyl)-3H-1,4-benzoxazepine (K.1.48);

### L) Biopesticides

L1) Microbial pesticides with fungicidal, bactericidal, viricidal and/or plant defense activator activity: Ampelomyces quisqualis, Aspergillus flavus, Aureobasidium pullulans, Bacillus altitudinis, B. amyloliquefaciens, B. megaterium, B. mojavensis, B. mycoides, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus alvei, Paenibacillus polymyxa, Pantoea vagans, Penicillium bilaiae, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperellum, T. atroviride, T. fertile, T. gamsii, T. harmatum, T. harzianum, T. polysporum, T. stromaticum, T. virens, T. viride, Typhula phacorrhiza, Ulocladium oudemansii, Vertialium dahlia, zucchini yellow mosaic virus (avirulent strain);
L2) Biochemical pesticides with fungicidal, bactericidal, viricidal and/or plant defense activator activity: chitosan (hydrolysate), harpin protein, laminarin, Menhaden fish oil, natamycin, Plum pox virus coat protein, potassium or sodium bicarbonate, Reynoutria sachalinensis extract, salicylic acid, tea tree oil;
L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia spp., Chromobacterium subtsugae, Cydia pomonella granulovirus (CpGV), Cryptophlebia leucotreta granulovirus (CrleGV), Flavobacterium spp., Helicoverpa armigera nucleopolyhedrovirus (HearNPV), Heterorhabditis bacteriophora, Isaria fumosorosea, Lecanicillium longisporum, L. muscarium, Metarhizium anisopliae, Metarhizium anisopliae var. anisopliae, M. anisopliae var. acridum, Nomuraea rileyi, Paecilomyces lilacinus, Paenibacillus popilliae, Pasteuria spp., P. nishizawae, P. penetrans, P. ramosa, P. thornea, P. usgae, Pseudomonas fluorescens, Spodoptera littoralis nucleopolyhedrovirus (SpliNPV), Steinernema carpocapsae, S. feltiae, S. kraussei, Streptomyces galbus, S. microflavus,
L4) Biochemical pesticides with insecticidal, acaricidal, molluscidal, pheromone and/or nematicidal activity: L-carvone, citral, (E,Z)-7,9-dodecadien-1-yl acetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-ylacetate, Z-7-tetradecen-2-one, Z-9-tetradecen-1-yl acetate, Z-11-tetradecenal, Z-11-tetradecen-1-ol, Acacia negra extract, extract of grapefruit seeds and pulp, extract of Chenopodium ambrosiodes, Catnip oil, Neem oil, Quillay extract, Tagetes oil;
L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium spp., B. elkanii, B. japonicum, B. liaoningense, B. lupini, Delfiia acidovorans, Glomus intraradices, Mesorhizobium spp., Rhizobium leguminosarum bv. phaseoli, R. l. bv. trifolii, R. l. bv. viciae, R. tropici, Sinorhizobium meliloti,
L6) Biochemical pesticides with plant stress reducing, plant growth regulator and/or plant yield enhancing activity: abscisic acid, aluminium silicate (kaolin), 3-decen-2-one, formononetin, genistein, hesperetin, homobrassinolide, humates, jasmonic acid and its salts or derivatives thereof, lysophosphatidyl ethanolamine, naringenin, polymeric polyhydroxy acid, Ascophyllum nodosum (Norwegian kelp, Brown kelp) extract and Ecklonia maxima (kelp) extract;

### M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl and uniconazole;

### N) Herbicides

acetamides: acetochlor (N.1.1), alachlor, butachlor, dimethachlor, dimethenamid (N.1.2), flufenacet (N.1.3), mefenacet (N.1.4), metolachlor (N.1.5), metazachlor (N.1.6), napropamide, naproanilide, pethoxamid, pretilachlor, propachlor, thenylchlor;
amino acid derivatives: bilanafos, glyphosate (N.2.1), glufosinate (N.2.2), sulfosate (N.2.3);
aryloxyphenoxypropionates: clodinafop (N.3.1), cyhalofop-butyl, fenoxaprop (N.3.2), fluazifop (N.3.3), haloxyfop (N.3.4), metamifop, propaquizafop, quizalofop, quizalofop-P-tefuryl;
Bipyridyls: diquat, paraquat (N.4.1);
(thio)carbamates: asulam, butylate, carbetamide, desmedipham, dimepiperate, eptam (EPTC), esprocarb, molinate, orbencarb, phenmedipham (N.5.1), prosulfocarb, pyributicarb, thiobencarb, triallate;
cyclohexanediones: butroxydim, clethodim (N.6.1), cycloxydim (N.6.2), profoxydim (N.6.3), sethoxydim (N.6.4), tepraloxydim (N.6.5), tralkoxydim;
dinitroanilines: benfluralin, ethalfluralin, oryzalin, pendimethalin (N.7.1), prodiamine (N.7.2), trifluralin (N.7.3);
diphenyl ethers: acifluorfen (N.8.1), aclonifen, bifenox, diclofop, ethoxyfen, fomesafen, lactofen, oxyfluorfen;
hydroxybenzonitriles: bomoxynil (N.9.1), dichlobenil, ioxynil;
imidazolinones: imazamethabenz, imazamox (N.10.1), imazapic (N.10.2), imazapyr (N.10.3), imazaquin (N.10.4), imazethapyr (N.10.5);
phenoxy acetic acids: clomeprop, 2,4-dichlorophenoxyacetic acid (2,4-D) (N.11.1), 2,4-DB, dichlorprop, MCPA, MCPA-thioethyl, MCPB, Mecoprop;
pyrazines: chloridazon (N.11.1), flufenpyr-ethyl, fluthiacet, norflurazon, pyridate;
pyridines: aminopyralid, clopyralid (N.12.1), diflufenican, dithiopyr, fluridone, fluroxypyr (N.12.2), picloram (N.12.3), picolinafen (N.12.4), thiazopyr;
sulfonyl ureas: amidosulfuron, azimsulfuron, bensulfuron (N.13.1), chlorinnuron-ethyl (N.13.2), chlorsulfuron, cinosulfuron, cyclosulfamuron (N.13.3), ethoxysulfuron, flazasulfuron, flucetosulfuron, flupyrsulfuron, foramsulfuron, halosulfuron, imazosulfuron, iodosulfuron (N.13.4), mesosulfuron (N.13.5), metazosulfuron, metsulfuron-methyl (N.13.6), nicosulfuron (N.13.7), oxasulfuron, primisulfuron, prosulfuron, pyrazosulfuron, rimsulfuron (N.13.8), sulfometuron, sulfosulfuron, thifensulfuron, triasulfuron, tribenuron, trifloxysulfuron, triflusulfuron (N.13.9), tritosulfuron, 1-((2-chloro-6-propyl-imidazo[1,2-b]pyridazin-3-yl)sulfonyl)-3-(4,6-dimethoxy-pyrimidin-2-yl)urea;
triazines: ametryn, atrazine (N.14.1), cyanazine, dimethametryn, ethiozin, hexazinone (N.14.2), metamitron, metribuzin, prometryn, simazine, terbuthylazine, terbutryn, triaziflam, trifludimoxazin (N14.3);
ureas: chlorotoluron, daimuron, diuron (N.15.1), fluometuron, isoproturon, linuron, methabenzthiazuron, tebuthiuron;
other acetolactate synthase inhibitors: bispyribac-sodium, cloransulam-methyl, diclosulam, florasulam (N.16.1), flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone (N.16.2), pyroxsulam;
others: amicarbazone, aminotriazole, anilofos, beflubutamid, benazolin, bencarbazone, benfluresate, benzofenap, bentazone (N.17.1), benzobicyclon, bicyclopyrone, bromacil, bromobutide, butafenacil, butamifos, cafenstrole, carfentrazone, cinidon-ethyl (N.17.2), chlorthal, cinmethylin (N.17.3), clomazone (N.17.4), cumyluron, cyprosulfamide, dicamba (N.17.5), difenzoquat, diflufenzopyr (N.17.6), Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac (N.17.7), quinmerac (N.17.8), mesotrione (N.17.9), methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil (N.17.10), sulcotrione (N.17.11), sulfentrazone, terbacil, tefuryltrione, tembotrione, thiencarbazone, topramezone (N.17.12), (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester;

### O) Insecticides

organo(thio)phosphates: acephate (O.1.1), azamethiphos (O.1.2), azinphos-methyl (O.1.3), chlorpyrifos (O.1.4), chlorpyrifos-methyl (O.1.5), chlorfenvinphos (O.1.6), diazinon (O.1.7), dichlorvos (O.1.8), dicrotophos (O.1.9), dimethoate (O.1.10), disulfoton (O.1.11), ethion (O.1.12), fenitrothion (O.1.13), fenthion (O.1.14), isoxathion (O.1.15), malathion (O.1.16), methamidophos (O.1.17), methidathion (O.1.18), methyl-parathion (O.1.19), mevinphos (O.1.20), monocrotophos (O.1.21), oxydemeton-methyl (O.1.22), paraoxon (O.1.23), parathion (O.1.24), phenthoate (O.1.25), phosalone (O.1.26), phosmet (O.1.27), phosphamidon (O.1.28), phorate (O.1.29), phoxim (O.1.30), pirimiphos-methyl (O.1.31), profenofos (O.1.32), prothiofos (O.1.33), sulprophos (O.1.34), tetrachlorvinphos (O.1.35), terbufos (O.1.36), triazophos (O.1.37), trichlorfon (O.1.38);
carbamates: alanycarb (O.2.1), aldicarb (O.2.2), bendiocarb (O.2.3), benfuracarb (O.2.4), carbaryl (O.2.5), carbofuran (O.2.6), carbosulfan (O.2.7), fenoxycarb (O.2.8), furathiocarb (O.2.9), methiocarb (O.2.10), methomyl (O.2.11), oxamyl (O.2.12), pirimicarb (O.2.13), propoxur (O.2.14), thiodicarb (O.2.15), triazamate (O.2.16);
pyrethroids: allethrin (O.3.1), bifenthrin (O.3.2), cyfluthrin (O.3.3), cyhalothrin (O.3.4), cyphenothrin (O.3.5), cypermethrin (O.3.6), alpha-cypermethrin (O.3.7), beta-cypermethrin (O.3.8), zeta-cypermethrin (O.3.9), deltamethrin (O.3.10), esfenvalerate (O.3.11), etofenprox (O.3.11), fenpropathrin (O.3.12), fenvalerate (O.3.13), imiprothrin (O.3.14), lambda-cyhalothrin (O.3.15), permethrin (O.3.16), prallethrin (O.3.17), pyrethrin I and II (O.3.18), resmethrin (O.3.19), silafluofen (O.3.20), tau-fluvalinate (O.3.21), tefluthrin (O.3.22), tetramethrin (O.3.23), tralomethrin (O.3.24), transfluthrin (O.3.25), profluthrin (O.3.26), dimefluthrin (O.3.27);
insect growth regulators: a) chitin synthesis inhibitors: benzoylureas: chlorfluazuron (O.4.1), cyramazin (O.4.2), diflubenzuron (O.4.3), flucycloxuron (O.4.4), flufenoxuron (O.4.5), hexaflumuron (O.4.6), lufenuron (O.4.7), novaluron (O.4.8), teflubenzuron (O.4.9), triflumuron (O.4.10); buprofezin (O.4.11), diofenolan (O.4.12), hexythiazox (O.4.13), etoxazole (O.4.14), clofentazine (O.4.15); b) ecdysone antagonists: halofenozide (O.4.16), methoxyfenozide (O.4.17), tebufenozide (O.4.18), azadirachtin (O.4.19); c) juvenoids: pyriproxyfen (O.4.20), methoprene (O.4.21), fenoxycarb (O.4.22); d) lipid biosynthesis inhibitors: spirodiclofen (O.4.23), spiromesifen (O.4.24), spirotetramat (O.4.24);
nicotinic receptor agonists/antagonists compounds: clothianidin (O.5.1), dinotefuran (O.5.2), flupyradifurone (O.5.3), imidacloprid (O.5.4), thiamethoxam (O.5.5), nitenpyram (O.5.6), acetamiprid (O.5.7), thiacloprid (O.5.8), 1-2-chloro-thiazol-5-ylmethyl)-2-nitrimino-3,5-dimethyl-[1,3,5]triazinane (O.5.9);
GABA antagonist compounds: endosulfan (O.6.19, ethiprole (O.6.2), fipronil (O.6.3), vaniliprole (O.6.4), pyrafluprole (O.6.5), pyriprole (O.6.6), 5-amino-1-(2,6-dichloro-4-methyl-phenyl)-4-sulfinamoyl-1H-pyrazole-3-carbothioic acid amide (O.6.7);
macrocyclic lactone insecticides: abamectin (O.7.1), emamectin (O.7.2), milbemectin (O.7.3), lepimectin (O.7.4), spinosad (O.7.5), spinetoram (O.7.6);
mitochondrial electron transport inhibitor (METI) I acaricides: fenazaquin (O.8.1), pyridaben (O.8.2), tebufenpyrad (O.8.3), tolfenpyrad (O.8.4), flufenerim (O.8.5);
METI II and III compounds: acequinocyl (O.9.1), fluacyprim (O.9.2), hydramethylnon (O.9.3);
Uncouplers: chlorfenapyr (O.10.1);
oxidative phosphorylation inhibitors: cyhexatin (O.11.1), diafenthiuron (O.11.2), fenbutatin oxide (O.11.3), propargite (O.11.4);
moulting disruptor compounds: cryomazine (O.12.1);
mixed function oxidase inhibitors: piperonyl butoxide (O.13.1);
sodium channel blockers: indoxacarb (O.14.1), metaflumizone (O.14.2);
ryanodine receptor inhibitors: chlorantraniliprole (O.15.1), cyantraniliprole (O.15.2), flubendiamide (O.15.3), N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.15.4); N-[4-chloro-2-[(di-ethyl-lambda-4-sulfanylidene)carbamoyl]-6-methyl-phenyl]-2-(3-chloro-2-pyridyl)-5-(trifluoromethyl)pyrazole-3-carboxamide (O.15.5); N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.15.6); N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.15.7); N-[4,6-dichloro-2-[(diethyl-lambda-4-sulfanylidene)carbamoyl]-phenyl]-2-(3-chloro-2-pyridyl)-5-(di-fluoromethyl)pyrazole-3-carboxamide (O.15.8); N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide (O.15.9); N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide (O.15.10); N-[4,6-dibromo-2-[(diethyl-lambda-4-sulfanylidene)carbamoyl]-phenyl]-2-(3-chloro-2-pyridyl)-5-(tri-fluoromethyl)pyrazole-3-carboxamide (O.15.11);
others: benclothiaz (O.16.1), bifenazate (O.16.2), artap (O.16.3), flonicamid (O.16.4), pyridalyl (O.16.5), pymetrozine (O.16.6), sulfur (O.16.7), thiocyclam (O.16.8), cyenopyrafen (O.16.9), flupyrazofos (O.16.10), cyflumetofen (O.16.11), amidoflumet (O.16.12), imicyafos (O.16.13), bistrifluron (O.16.14), pyrifluquinazon (O.16.15) and 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]cyclopropaneacetic acid ester (O.16.16).

The present invention furthermore relates to agrochemical compositions comprising a mixture of at least one compound I (component 1) and at least one further active substance useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e. g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day. In case of a mixture comprising a pesticide II selected from group L), it is preferred that the pesticide II is applied as last treatment.

According to the invention, the solid material (dry matter) of the biopesticides (with the exception of oils such as Neem oil, Tagetes oil, etc.) are considered as active components (e. g. to be obtained after drying or evaporation of the extraction medium or the suspension medium in case of liquid formulations of the microbial pesticides).

In accordance with the present invention, the weight ratios and percentages used herein for a biological extract such as Quillay extract are based on the total weight of the dry content (solid material) of the respective extract(s).

The total weight ratios of compositions comprising at least one microbial pesticide in the form of viable microbial cells including dormant forms, can be determined using the amount of CFU of the respective microorganism to calculate the total weight of the respective active component with the following equation that 1×10¹⁰ CFU equals one gram of total weight of the respective active component. Colony forming unit is measure of viable microbial cells, in particular fungal and bacterial cells. In addition, here “CFU” may also be understood as the number of (juvenile) individual nematodes in case of (entomopathogenic) nematode biopesticides, such as Steinernema feltiae.

In the binary mixtures and compositions according to the invention the weight ratio of the component 1) and the component 2) generally depends from the properties of the active components used, usually it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to further embodiments of the binary mixtures and compositions, the weight ratio of the component 1) and the component 2) usually is in the range of from 1000:1 to 1:1, often in the range of from 100: 1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

According to further embodiments of the mixtures and compositions, the weight ratio of the component 1) and the component 2) generally depends from the properties of the active components used, usually it is in the range of from 1:10,000 to 10,000:1, regularly in the range of from 1:100 to 10,000:1, preferably in the range of from 1:100 to 5,000:1, more preferably in the range of from 1:1 to 1,000:1, even more preferably in the range of from 1:1 to 500:1 and in particular in the range of from 10:1 to 300:1.

According to further embodiments of the mixtures and compositions, the weight ratio of the component 1) and the component 2) usually is in the range of from 20,000:1 to 1:10, often in the range of from 10,000:1 to 1:1, regularly in the range of from 5,000:1 to 5:1, preferably in the range of from 5,000:1 to 10:1, more preferably in the range of from 2,000:1 to 30:1, even more preferably in the range of from 2,000:1 to 100:1 and in particular in the range of from 1,000:1 to 100:1.

According to further embodiments of the mixtures and compositions, the weight ratio of the component 1) and the component 2) usually is in the range of from 1:20,000 to 10:1, often in the range of from 1:10,000 to 1:1, regularly in the range of from 1:5,000 to 1:5, preferably in the range of from 1:5,000 to 1:10, more preferably in the range of from 1:2,000 to 1:30, even more preferably in the range of from 1:2,000 to 1:100 and in particular in the range of from 1:1,000 to 1:100.

In the ternary mixtures, i.e. compositions according to the invention comprising the component 1 and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

When mixtures comprising microbial pesticides are employed in crop protection, the application rates preferably range from about 1×10⁶ to 5×10¹⁵ (or more) CFU/ha, preferably from about 1×10⁸ to about 1×10¹³ CFU/ha, and even more preferably from about 1×10⁹ to about 1×10¹² CFU/ha. In the case of (entomopathogenic) nematodes as microbial pesticides (e. g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e. g. in the form of eggs, juvenile or any other live stages, preferably in an infetive juvenile stage) per ha.

When mixtures comprising microbial pesticides are employed in seed treatment, the application rates with respect to plant propagation material preferably range from about 1×10⁶ to 1×10¹² (or more) CFU/seed. Preferably, the concentration is about 1×10⁶ to about 1×10⁹ CFU/seed. In the case of the microbial pesticides II, the application rates with respect to plant propagation material also preferably range from about 1×10⁷ to 1×10¹⁴ (or more) CFU per 100 kg of seed, preferably from 1×10⁹ to about 1×10¹² CFU per 100 kg of seed.

Preference is also given to mixtures comprising as component 2) at least one active substance selected from group A), which is particularly selected from (A.1.1), (A.1.4), (A.1.8), (A.1.9), (A.1.12), (A.1.13), (A.1.14), (A.1.17), (A.1.19), (A.1.21), (A.2.1), (A.2.2), (A.3.2), (A.3.3), (A.3.4), (A.3.7), (A.3.8), (A.3.9), (A.3.12), (A.3.14), (A.3.15), (A.3.16), (A.3.19), (A.3.20), (A.3.21), (A.3.22), (A.3.23), (A.3.24), (A.3.25), (A.3.26), (A.3.27); (A.4.5), (A.4.6), (A.4.8), (A.4.9), (A.4.11), (A.1.23), (A.1.24) (A.1.25) and (A.1.26). In certain embodiments component 2 is selected from azoxystrobin, dimoxystrobin, fluoxastrobin, kresoxim-methyl, orysastrobin, picoxystrobin, pyraclostrobin, trifloxystrobin; famoxadone, fenamidone; benzovindiflupyr, bixafen, boscalid, fluopyram, fluxapyroxad, isopyrazam, penflufen, penthiopyrad, sedaxane; ametoctradin, cyazofamid, fluazinam, fentin salts, such as fentin acetate.

Preference is given to mixtures as component 2) at least one active substance selected from group B), which is particularly selected from (B.1.4), (B.1.5), diniconazole (B.1.6), (B.1.8), (B.1.10), (B.1.11), (B.1.12), (B.1.17), (B.1.18), (B.1.21), (B.1.22), (B.1.23), (B.1.25), (B.1.26), (B.1.27), (B.1.28), (B.1.29), uni (B.1.31), (B.1.32), (B.1.33), (B.1.34), (B.1.35), (B.1.36), (B.1.37), (B.1.38), (B.1.39), (B.1.40), (B.1.41), (B.1.42), (B.1.44), (B.1.46), (B.1.49) and (B.1.50; (B.2.2), (B.2.4), (B.2.5), (B.2.6), piperalin (B.2.7), (B.2.8); and (B.3.1). In certain embodiments component 2 is selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, fenarimol, triforine; dodemorph, fenpropimorph, tridemorph, fenpropidin, spiroxamine; fenhexamid.

Preference is given to mixtures comprising as component 2) at least one active substance selected from group C), which is particularly selected from (C.1.4), C.1.5), (C.1.6), and (C.2.4).

In certain embodiments,

component 2 is selected from metalaxyl, (metalaxyl-M) mefenoxam, ofurace.

Preference is given to mixtures comprising as component 2) at least one active substance selected from group D), which is particularly selected from (D1.1), (D1.2), (D1.4), (D1.5); (D2.2), (D2.4), (D2.5), (D2.6) and (D2.7). In certain embodiments component 2 is selected from benomyl, carbendazim, thiophanate-methyl, ethaboxam, fluopicolide, zoxamide, metrafenone, pyriofenone.

Preference is also given to mixtures comprising as component 2) at least one active substance selected from group E), which is particularly selected from (E.1.1), (E.1.2), and (E.1.3).

In certain embodiments component 2 is selected from cyprodinil, mepanipyrim, pyrimethanil.

Preference is also given to mixtures comprising as component 2) at least one active substance selected from group F), which is particularly selected from (F.1.2), (F.1.4), (F.1.5), (F.1.6) and (F.2.1). In certain embodiments component 2) is selected from iprodione, fludioxonil, vinclozolin, quinoxyfen.

Preference is also given to mixtures as component 2) at least one active substance selected from group G), which is particularly selected from (G.3.1), (G.3.2), (G.3.3), (G.3.4), (G.3.5), (G.3.6), (G.4.1) and (G.5.1).

In certain embodiments component 2 is selected from dimethomorph, flumorph, iprovalicarb, benthiavalicarb, mandipropamid, propamocarb.

Preference is also given to mixtures comprising as component 2) at least one active substance selected from group H), which is and particularly selected from (H.1.2), (H.1.3), copper oxychloride (H.1.4), (H.1.5), (H.1.6); (H.2.2), (H.2.5), (H.2.7), (H.3.2), (H.3.3), (H.3.4), (H.3.5), (H.3.6), (H.3.12); (H.4.2), (H.4.6), dithianon (H.4.9) and (H.4.10). In certain embodiments, component 2 is selected from copper acetate, copper hydroxide, copper oxychloride, copper sulfate, sulfur, mancozeb, metiram, propineb, thiram, captafol, folpet, chlorothalonil, dichlofluanid, dithianon.

Preference is also given to mixtures comprising as component 2) at least one active substance selected from group I), which is particularly selected from (I.2.3) and (I.2.5).

In certain embodiments, component 2 is selected from carpropamid and fenoxanil.

Preference is also given to mixtures comprising as component 2) at least one active substance selected from group J), which is particularly selected from (J.1.1), (J.1.2), (J.1.3), (J.1.4), (J.1.6), (J.1.7), (J.1.8) and (J.1.9). In certain embodiments, component 2 is selected from acibenzolar-S-methyl, probenazole, tiadinil, fosetyl, fosetyl-aluminium, H₃PO₃ and salts thereof.

Preference is also given to mixtures comprising as component 2) at least one active substance selected from group K), which is particularly selected from (K.1.4), (K.1.5), (K.1.8), (K.1.12), (K.1.14), (K.1.15), (K.1.19) and (K.1.22). In certain embodiments component 2 is selected from cymoxanil, proquinazid and N-methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide.

The biopesticides from group L1) and/or L2) may also have insecticidal, acaricidal, molluscidal, pheromone, nematicidal, plant stress reducing, plant growth regulator, plant growth promoting and/or yield enhancing activity. The biopesticides from group L3) and/or L4) may also have fungicidal, bactericidal, viricidal, plant defense activator, plant stress reducing, plant growth regulator, plant growth promoting and/or yield enhancing activity. The biopesticides from group L5) and/or L6) may also have fungicidal, bactericidal, viricidal, plant defense activator, insecticidal, acaricidal, molluscidal, pheromone and/or nematicidal activity.

Many of these biopesticides have been deposited under deposition numbers mentioned herein (the prefices refer to the acronym of the respective culture collection), are referred to in literature, registered and/or are commercially available: aluminium silicate (Screen™ Duo from Certis LLC, USA), Agrobacterium radiobacter K1026 (e. g. NoGall® from BASF Agricultural Specialties Pty Ltd, Australia), A. radiobacter K84 (Nature 280, 697-699, 1979; e. g. GallTroll® from AG Biochem, Inc., C, USA), Ampelomyces quisqualis M-10 (e. g. AQ 10® from Intrachem Bio GmbH & Co. KG, Germany), Ascophyllum nodosum (Norwegian kelp, Brown kelp) extract or filtrate (e. g. ORKA GOLD from BASF Agricultural Specialties (Pty) Ltd., South Africa; or Goemar® from Laboratories Goemar, France), Aspergillus flavus NRRL 21882 isolated from a peanut in Georgia in 1991 by USDA, National Peanut Research Laboratory (e. g. in Afla-Guard® from Syngenta, CH), mixtures of Aureobasidium pullulans DSM 14940 and DSM 14941 (e. g. blastospores in Blossom Protect® from bio-ferm GmbH, Germany), Azospirillum amazonense SpY2 (DN: BR 11140; Proc. 9^{th} Int. and 1^{st} Latin American PGPR meeting, Quimara, Medellín, Colombia 2012, p. 60, ISBN 978-958-46-0908-3), A. brasilense AZ39 (also called Az 39; INTA Az-39; Eur. J. Soil Biol 45(1), 28-35, 2009), A. brasilense XOH (e. g. AZOS from Xtreme Gardening, USA or RTI Reforestation Technologies International; USA), A. brasilense BR 11002 (Proc. 9th Int. and 1^{st} Latin American PGPR meeting, Quimara, Medellín, Colombia 2012, p. 60, ISBN 978-958-46-0908-3), A. brasilense Sp245 (BR 11005; e. g. in GELFIX Gramíneas from BASF Agricultural Specialties Ltd., Brazil), A. brasilense strains Ab-V5 and Ab-V6 (e. g. in AzoMax from Novozymes BioAg Produtos papra Agricultura Ltda., Quattro Barras, Brazil or SimbioseMaíz® from Simbiose-Agro, Cruz Alta, RS, Brazil; Plant Soil 331, 413-425, 2010), A. lipoferum BR 11646 (Sp31) (Proc. 9th Int. and 1^{st} Latin American PGPR meeting, Quimara, Medellín, Colombia 2012, p. 60), Bacillus altitudinis 41KF2b (DSM 21631; Int. J. Syst. Evol. Microbiol. 56(7), 1465-1473, 2006), Bacillus amyloliquefaciens strains AP-136 (NRRL B-50614 and B-50330), AP-188 (NRRL B-50615 and B-50331), AP-218 (NRRL B-50618), AP-219 (NRRL B-50619 and B-50332), and AP-295 (NRRL B-50620 and B-50333) all known from U.S. Pat. No. 8,445,255; B. amyloliquefaciens IT-45 (CNCM I-3800) (e. g. Rhizocell C from ITHEC, France), B. amyloliquefaciens IN937a (J. Microbiol. Biotechnol. 17(2), 280-286, 2007; e. g. BioYield® from Gustafson LLC, TX, USA), B. amyloliquefaciens spp. plantarum D747 (US 20130236522 A1; FERM BP-8234; e. g. Double Nickel™ 55 WDG or Double Nickel™ LC from Certis LLC, USA), B. amyloliquefaciens spp. plantarum FZB24 isolated from plant pathogen-infested soil of a sugar beet field in Brandenburg, Germany (also called SB3615; DSM ID 96-2; J. Plant Dis. Prot. 105, 181-197, 1998; e. g. Taegro® from Novozyme Biologicals, Inc., USA),), B. amylo-liquefaciens spp. plantarum SB3615vPPI being a phage-resistant variant of FZB24 (MRRL B-50349; US 2011/023045 A1; from Novozyme Biologicals, Inc., USA), B. amyloliquefaciens ssp. plantarum FZB42 isolated from plant pathogen-infested soil of a sugar beet field in Brandenburg, Germany (J. Plant Dis. Prot. 105, 181-197, 1998; DSM 23117; e. g. RhizoVital® 42 from AbiTEP GmbH, Berlin, Germany), B. amyloliquefaciens ssp. plantarum GB03 (also called GBO3; ATCC SD-1397; Phytopathol. 86(11), S36, 1996; e. g. Kodiak® or BioYield® from Gustafson, Inc., USA; or Companion® from Growth Products, Ltd., White Plains, N.Y. 10603, USA), B. amyloliquefaciens ssp. plantarum MBI600 also referred to as 1430 (NRRL B-50595; Int. J. Microbiol. Res. 3(2) (2011), 120-130; US 2012/0149571 A1; e. g. Integral®, Subtilex® NG from BASF Corp., USA), B. amyloliquefaciens spp. plantarum TJ1000 (also called 1BE; CA 2471555 A1; ATCC BAA-390; e. g. QuickRoots™ from TJ Technologies, Watertown, S. Dak., USA), B. cereus CNCM I-1562 (U.S. Pat. No. 6,406,690), B. chitinosporus AQ746 isolated from roots in Saskatchewan, Canada (NRRL B-21618; U.S. Pat. No. 5,733,544; AgraQuest now Bayer CropScience LP, USA), B. firmus CNCM I-1582 (WO 2009/126473, WO 2009/124707, U.S. Pat. No. 6,406,690; e. g. Votivo® from Bayer CropScience LP, USA), B. megaterium strains H491 (NRRL B-50769), M018 (NRRL B-50770) and J142 (NRRL B-50771) all known from US 2014/0051571 A1 from Marrone BioInnovations, Inc., USA; B. mojavensis AP-209 (NRRL B-50616; U.S. Pat. No. 8,445,255), B. mycoides AQ726 (NRRL B-21664; U.S. Pat. No. 5,906,818; from Bayer Crop Science, Germany), B. mycoides strain J (e.g. BmJ WG from Certis, USA against potato virus Y), B. pumilus GB34 (ATCC 700814; e. g. YieldShield® from Gustafson LLC, TX, USA), B. pumilus GHA 180 isolated from apple tree rhizosphere in Mexico (IDAC 260707-01; e. g. in PRO-MIX® BX from Premier Horticulture, 1, avenue Premier, Rivie're-du-Loup, Quebec, Canada G5R6C1), B. pumilus KFP9F (NRRL B-50754; WO 2014/029697; e. g. BAC-UP or FUSION-P from BASF Agricultural Specialties (Pty) Ltd., South Africa), B. pumilus INR-7 otherwise referred to as BU-F22 and BU-F33 (NRRL B-50185, NRRL B-50153; U.S. Pat. No. 8,445,255), B. pumilus QST 2808 (NRRL B-30087; e. g. Sonata® or Ballad® Plus from AgraQuest Inc., USA), B. solisalsi AP-217 (NRRL B-50617; U.S. Pat. No. 8,445,255), B. subtilis CX-9060 (Federal Register 77(7), 1633-1637; by Certis U.S.A., L.L.C.), B. subtilis FB17 also called UD 1022 or UD10-22 isolated from red beet roots in North America (ATCC PTA-11857; System. Appl. Microbiol. 27, 372-379, 2004; US 2010/0260735; WO 2011/109395); B. subtilis GB07 (Phytopathol. 86(11), S36, 1996; Epic® from Gustafson, Inc., USA), B. subtilis QST-713 isolated from a California peach orchard in 1995 (NRRL B-21661; e. g. Rhapsody®, Serenade® MAX or Serenade® ASO from AgraQuest Inc., USA), B. thuringiensis ssp. aizawai ABTS-1857 (also called ABG-6346; ATCC SD-1372; e. g. XenTari® from BioFa AG, Münsingen, Germany), B. t. ssp. aizawai SAN 401 I, ABG-6305 (WO 2013/087709); Bacillus t. ssp. israelensis AM65-52 of Serotype H-14 (ATCC SD-1276; e. g. VectoBac® from Valent BioSciences, IL, USA), Bacillus thuringiensis ssp. kurstaki SB4 (NRRL B-50753; e. g. Beta Pro® from BASF Agricultural Specialties (Pty) Ltd., South Africa), B. t. ssp. kurstaki ABTS-351 identical to HD-1 (ATCC SD-1275; e. g. Dipel® DF from Valent BioSciences, IL, USA), B. t. ssp. kurstaki EG 2348 (NRRL B-18208; e. g. Lepinox® or Rapax® from CBC (Europe) S.r.I., Italy), B. t. ssp. tenebrionis DSM 2803 of Serotype H 8a, 8b (identical to NRRL B-15939; EP 0 585 215 B1; Mycogen Corp.), B. t. ssp. tenebrionis NB-125 (also referred to as SAN 418 I or ABG-6479; EP 0 585 215 B1; DSM 5526; former production strain of Novo-Nordisk), B. t. ssp. tenebrionis NB-176 (or NB-176-1; a gamma-irradiated, induced high-yielding mutant of strain NB-125; EP 585 215 B1; DSM 5480; e. g. Novodor® from Valent BioSciences, Switzerland), Beauveria bassiana JW-1 (ATCC 74040; e. g. Naturalis® from CBC (Europe) S.r.I., Italy), B. bassiana DSM 12256 (US 200020031495; e. g. BioExpert® SC from Live Sitemaps Technology S.A., Colombia), B. bassiana GHA (ATCC 74250; e. g. BotaniGard® 22WGP from Laverlam Int. Corp., USA), B. bassiana PPRI 5339 (ARSEF 5339; NRRL 50757; e. g. BroadBand® from BASF Agricultural Specialties (Pty) Ltd., South Africa), B. brongniartii for control of cockchafer (J. Appl. Microbiol. 100(5), 1063-72, 2006; e. g. Melocont® from Agrifutur, Agrianello, Italy), Bradyrhizobium sp. (e. g. Vault® from BASF Corp., USA), B. sp. (Arachis) CB1015 presumably originally collected in India (IITA 1006, USDA 3446; from Australian Inoculants Research Group; http://www.qaseeds.com.au/inoculant_applic.php). B. sp. (Arachis) strains deposited at SEMIA and known from FEMS Microbiol. Letters 303(2), 123-131, 2010; Revista Brasileira de Ciencia do Solo 35(3), 739-742, 2011, ISSN 0100-0683: SEMIA 6144, SEMIA 6462 (BR 3267) and SEMIA 6464 (BR 3262); B. sp. (Vigna) PNLO1 (Bisson and Mason, Apr. 29, 2010, Project report, Worcester Polytechnic Institute, Worcester, Mass., USA: http://www.wpi.edu/Pubs/E-project/Available/E-project-042810-163614/; e. g. Vault® Peanut Liquid from BASF Corp., USA), B. elkanii SEMIA 587 (Appl. Environ. Microbiol. 73(8), 2635, 2007; e. g. GELFIX 5 from BASF Agricultural Specialties Ltd., Brazil), B. elkanii SEMIA 5019 (=29W; Appl. Environ. Microbiol. 73(8), 2635, 2007; e. g. GELFIX 5 from BASF Agricultural Specialties Ltd., Brazil), B. elkanii USDA 76, B. elkanii USDA 94B. elkanii USDA 3254, B. elkanii U-1301 and U-1302 (e. g. Nitragin® Optimize from Novozymes Bio As S.A., Brazil, or Nlitrasec for soybean from LAGE y Cia, Brazil), B. japonicum (e. g. VAULT® from BASF Corp., USA), B. japonicum 532c isolated from Wisconsin field (Nitragin 61A152; Can. J. Plant. Sci. 70, 661-666, 1990; e. g. in Rhizoflo®, Histick®, Hicoat® Super from BASF Agricultural Specialties Ltd., Canada), B. japonicum E-109 variant of strain USDA 138 (INTA E109, SEMIA 5085; Eur. J. Soil Biol. 45, 28-35, 2009; Biol. Fertil. Soils 47, 81-89, 2011), B. japonicum G49 (MSDJ G49; C. R. Acad. Agric. Fr. 73, 163-171, 1987); B. japonicum strains deposited at SEMIA known from Appl. Environ. Microbiol. 73(8), 2635, 2007: SEMIA 566 isolated from North American inoculant in 1966 and used in Brazilian commercial inoculants from 1966 to 1978, SEMIA 586 originally isolated in Maryland, USA, in 1961 but received from Australia in 1966 and used in Brazilian inoculants in 1977 (CB 1809, USDA 136, Nitragin 61A136, RCR 3407), SEMIA 5079 a natural variant of SEMIA 566 used in commercial inoculants since 1992 (CPAC 15; e. g. GELFIX 5 or ADHERE 60 from BASF Agricultural Specialties Ltd., Brazil), B. japonicum SEMIA 5080 a natural variant of SEMIA 586 used in commercial inoculants since 1992 (CPAC 7; e. g. GELFIX 5 or ADHERE 60 from BASF Agricultural Specialties Ltd., Brazil); B. japonicum TA-11 (TA11 NOD⁺) (NRRL B-18466; U.S. Pat. No. 5,021,076; Appl. Environ. Microbiol. 56, 2399-2403, 1990; e. g. VAULT® NP, from BASF Corp., USA), B. japonicum strains deposited at USDA known from U.S. Pat. No. 7,262,151 and Appl. Environ. Microbiol. 60, 940-94, 1994: USDA 3 isolated from Glycine max in Virginia (USA) in 1914, USDA 31 (=Nitragin 61A164) od Serogroup 31 isolated from Glycine max in Wisconsin (USA) in 1941, USDA 76 isolated from plant passage of strain USDA 74 (Serogroup 76) which has been isolated from G. max in California (USA) in 1956, USDA 110 (=IITA 2121, SEMIA 5032, RCR 3427, ARS I-110 and Nitragin 61A89; Serogroup 110) isolated from G. max in Florida in 1959, USDA 121 isolated from G. max in Ohio (USA) in 1965 (Crop Science 26(5), 911-916, 1986); B. japonicum WB74 (e. g. Eco-Rhiz Soya from Plant Health Products (Pty) Ltd, South Africa; or Soybean inoculant from Stimuplant CC, South Africa), B. lupini LL13 isolated from Lupinus iuteus nodules from French soils (deposited at INRA, France; http://agriculture.gouv.fr/IMG/pdf/ch20060216.pdf), B. lupini strains from Australia and known from Palta J. A., Berger J. B. (eds), Proceed. 12th International Lupin Conference, 14-18 Sep. 2008, Fremantle, Western Australia, International Lupin Association, Canterbury, New Zealand, 47-50, http://www.lupins.org/pdf/conference/2008/Agronomy%20and %20Production/John%20Howieson%20and %20G % 20OHara.pdf; Appl. Environ. Microbiol. 71, 7041-7052, 2005; Australian J. Exp. Agricult. 36(1), 63-70, 1996: strains WU425 isolated in Esperance, Western Australia from a non-Australian legume Ornithopus compressus, WSM471 isolated from Ornithopus pinnatus in Oyster Harbour, Western Australia, and WSM4024 isolated from lupins in Australia by CRS during a 2005 survey; Burkholderia sp. A396 (NRRL B-50319; WO 2013/032693; Marrone Bio Innovations, Inc., USA), Candida oleophila I-182 (NRRL Y-18846; Phytoparasitica 23(3), 231-234, 1995; e. g. Aspire® from Ecogen Inc., USA;), C. oleophila strain O (NRRL Y-2317; Biological Control 51, 403-408, 2009), Candida saitoana (e. g. Biocure® [in mixture with lysozyme] and BioCoat® from Micro Flo Company, USA (BASF SE) and Arysta), chitosan (e. g. Armour-Zen® from BotriZen Ltd., NZ), Clonostachys rosea f. catenulate (also named Gliocladium catenulatum) J1446 isolated from Finnish field soil (NJF seminar No 389: Pest, disease and weed management in strawberry; Finland 8-9. November 2006 in NJF Report 2(10), 15-15, 2006; DSM 9212; e. g. Primastop® or Prestop® from Verdera Oy, Finland), Chromobacterium subtsugae PRAA4-1 isolated from soil under an eastern hemlock (Tsuga canadensis) in the Catoctin Mountain region of central Maryland (NRRL B-30655; e. g. Grandevo® from Marrone Bio Innovations, USA), Coniothyrium minitans CON/M/91-08 (WO 1996/021358; DSM 9660; e. g. Contans® WG, Intercept® WG from Prophyta Biologischer Pflanzenschutz GmbH, Germany), Cryphonectria parasitica (hypovirulent strains; Microbiol. Reviews 56(4), 561-576, 1992; e. g. product Endothia parasitica from CNICM, France), Cryptococcus albidus (e. g. YIELD PLUS® from Anchor Bio-Technologies, South Africa), Cryptophlebia leucotreta granulovirus (CrleGV) (e. g. CRYPTEX from Adermatt Biocontrol, Switzerland), Cydia pomonella granulovirus (CpGV) V03 (DSM GV-0006; e. g. Madex® Max from Andermatt Biocontrol, Switzerland), CpGV V22 (DSM GV-0014; e. g. Madex® Twin from Adermatt Biocontrol, Switzerland), Delftia acidovorans RAY209 (ATCC PTA-4249; WO 2003/57861; e. g. BioBoost® from Brett Young, Winnipeg, Canada), Dilophosphora alopecuri (FarmNote 396, February 2010, Department of Agriculture and Food, Government of Western Australia; e.g. Twist Fungus from BASF Agricultural Specialties Pty Ltd, Australia), Ecklonia maxima (kelp) extract (J. Ecological Engineering 14(1), 48-52, 2013; e. g. KELPAK SL from Kelp Products Ltd, South Africa), Flavobacterium sp. H492 (ATCC B-505584; WO 2013/138398; e. g. MBI-302 from Marrone Bio Innovations, USA for soyean cyst nematode control), formononetin (U.S. Pat. No. 5,002,603; e. g. Myconate® from Plant Health Care plc, U.K.), Fusarium oxysporum Fo47 (non-pathogenic strain isolated from a suppressive soil located at Châteaurenard, France; Appl. Environ. Microbiol 68(8), 4044-4060, 2002; Fusaclean® from Natural Plant Protection, N.P.P. (sociétéanonyme) Route d'Artix F-64150 Nogueres, France), F. oxysporum 251/2RB (Prevention Today Vol. 2, n. 1-2, 47-62, 2006; e. g. Biofox® C from S.I.A.P.A., Italy); Glomus intraradices (e. g. Myc® 4000 from ITHEC, France), Glomus intraradices RTI-801 (e. g. MYKOS from Xtreme Gardening, USA or RTI Reforestation Technologies International; USA), grapefruit seeds and pulp extract (e. g. BC-1000 from Chemie S. A., Chile), harpin (alpha-beta) protein (Science 257, 85-88, 1992; e. g. Messenger™ or HARP-N-Tek from Plant Health Care plc, U.K.), Helicoverpa armigera nucleopolyhedrovirus (HearNPV) (J. Invertebrate Pathol. 107, 112-126, 2011; e. g. Helicovex® from Adermatt Biocontrol, Switzerland), Heterorhabditis bacteriophora (e. g. Nemasys® G from BASF Agricultural Specialities Limited, UK), Isaria fumosorosea Apopka-97 (ATCC 20874; Biocontrol Science Technol. 22(7), 747-761, 2012; e. g. PFR-97™ or PreFeRal® from Certis LLC, USA), I. fumosorosea FE 9901 (ARSEF 4490; Biocontrol Science Technol. 22(7), 747-761, 2012; e. g. blastospores in NoFly™ WP from Natural Industries, Inc., Houston, Tex., USA or from Novozymes, U.S.A.), cis-jasmone (U.S. Pat. No. 6,890,525; U.S. Pat. No. 8,221,736; Plant Bioscience Limited, Norwich, U.K.), laminarin (e. g. in Vacciplant® from Laboratoires Goemar, St. Malo, France or Stähler SA, Switzerland), Lecanicillium longisporum KV42 and KV71 (e. g. Vertalec® from Koppert BV, Netherlands), L. muscarium Ve6 (also called KV01; —IMI 19-79, CABI 268317, CBS 102071, ARSEF 5128; e. g. Mycotal® from Koppert BV, Netherlands), Lysobacter antibioticus 13-1 (Biological Control 45, 288-296, 2008), L. antibioticus HS124 (Curr. Microbiol. 59(6), 608-615, 2009), L. enzymogenes 3.1T8 (Microbiol. Res. 158, 107-115, 2003; Biological Control 31(2), 145-154, 2004); Mesorhizobium spp. strains known from Soil Biol. Biochem. 36(8), 1309-1317, 2004; Plant and Soil 348(1-2), 231-243, 2011: M. sp. WSM1271 collected in Sardinia, Italy, from plant host Biserrula pelecinus, M. sp. WSM 1497 collected in Mykonos, Greece, from Biserrula pelecinus, Mesorhizobium ciceri CC1192 collected in Israel from Cicer arietinum nodules (UPM 848, CECT 5549; Can. J. Microbiol. 48, 279-284, 2002; from Horticultural Research Station, Gosford, Australia), M. huakuii HN3015 isolated from Astralagus sinicus in a rice-growing field of Southern China (World J. Microbiol. Biotechn. 23(6), 845-851, 2007, ISSN 0959-3993), M. loti CC829 isolated from L. ulginosus nodules in USA (NZP 2012; commercial inoculant for Lotus pedunculatus and L. ulginosus in Australia), and M. loti SU343 isolated from host nodules in USA (commercial inoculant for Lotus corniculatus in Australia); Metarhizium anisopliae FI-1045 (AGAL V10/0104285; WO 2012/018266; e. g. Biocane® from BASF Agricultural Specialties Pty Ltd, Australia), M. anisopliae var. anisopliae F52 also called 275 or V275 (DSM 3884, ATCC 90448; e. g. Met52® Novozymes Biologicals BioAg Group, Canada), M. anisopliae ICIPE 69 isolated from a soil sample obtained from the Democratic Republic of Congo (DRC) and using the Galleria bait method in 1990 (e. g. Metathripol from ICIPE, Nairobe, Kenya), M. anisopliae var. acridum IMI 330189 isolated from Ornithacris cavroisi in Niger (NRRL 50758; e. g. Green Muscle® from BASF Agricultural Specialities (Pty) Ltd., South Africa), M. a. var. acridum FI-985 isolated from a spur-throated locust, Austracris guttulosa (Walker), near Rockhampton, Queensland, Australia, in 1979 (ARSEF 324; Memoirs of the Entomological Society of Canada 171, 287-300, 1997; e. g. Green Guard® SC from BASF Agricultural Specialties Pty Ltd, Australia), Metschnikowia fructicola 277 isolated from the surface of grape berries (cv. Superior) grown in the central part of Israel (U.S. Pat. No. 6,994,849; NRRL Y-30752; e. g. Shemer® from Agrogreen, Israel, now distributed by Bayer CropSciences, Germany), Microdochium dimerum L13 (CNCM I-3141; e. g. Antibot® from Agrauxine, France), Microsphaeropsis ochracea P130A isolated from apple leaves from an abandoned orchard, St-Joseph-du-Lac, Quebec, Canada in 1993 (ATCC 74412; Mycologia 94(2), 297-301, 2002), Muscodor albus QST 20799 also called 620 originally isolated from the bark of a cinnamon tree in Honduras (NRRL 30547; e. g. Muscudor™ or QRD300 from AgraQuest, USA), Muscodor albus SA-13 (NRRL B-50774; US 2014/0086879 A1; e. g. MBI-601-EP from Marrone BioInnovations, Inc., USA), Neem oil (e. g. Trilogy®, Triact® 70 EC from Certis LLC, USA), Nomuraea rileyi strains SA86101, GU87401, SR86151, CG128 and VA9101 (Braz. Arch. Biol. Technol. 46(1), 13-19, 2003; WO 2013/110594), Paecilomyces lilacinus 251 isolated from infected nematode eggs in the Philippines (AGAL 89/030550; WO1991/02051; Crop Protection 27, 352-361, 2008; e. g. BioAct®/MeloCon® from Prophyta, Germany), P. lilacinus DSM 15169 (e. g. Nemata® SC from Live Systems Technology S.A., Colombia), P. lilacinus BCP2 (NRRL 50756; Acta agriculturae Slovenica, 101-2, 263-275, 2013; e. g. PL Gold from BASF Agricultural Specialities (Pty) Ltd., South Africa), Paenibacillus alvei NAS6G6 (WO 2014/029697; NRRL B-50755; e.g. BAC-UP from BASF Agricultural Specialities (Pty) Ltd., South Africa in mixture with Bacillus pumilus KFP9F), P. polymyxa PKB1 (ATCC 202127; Can. J. Microbiol. 48(2), 159-169, 2002), Pantoea agglomerans E325 (NRRL B-21856; Phytopathol. 101(10), 1234-41, 2011; Trees 26, 227-238, 2012; Bloomtime Biological™ from Northwest Agricultural Products, Inc., USA), Pantoea vagans (formerly agglomerans) C9-1 originally isolated in 1994 from apple stem tissue for control of fire blight in apple (J. Bacteriol. 192(24), 6486-6487, 2010; e. g. BlightBan C9-10 from NuFrams America Inc., USA), Pasteuria sp. ATCC PTA-9643 (WO 2010/085795), Pasteuria sp. Ph3 isolated from turfgrass soil samples collected at the DeBary Golf Course in central Florida (ATCC SD-5832; WO 2012/064527; for control of Hoplolaimus galeatus nematode from Pasteuria Bioscience, Inc. now Syngenta Crop Protection, LLC, USA), Pasteuri asp. Pr3 isolated from soil samples collected in the south-eastern United States (ATCC SD-5834; for control of Rotylenchulus reniformis nematode potentially of species P. ramosa; Naviva® ST from Syngenta Crop Protection, LLC, USA), P. nishizawae (WO 2010/80619), P. nishizawae Pn1 (Federal Register 76(22), 5808, Feb. 2, 2011; ATCC SD-5833; e.g. Clariva™ PN from Syngenta Crop Protection, LLC, USA), P. penetrans (U.S. Pat. No. 5,248,500; Del Monte Corp.), P. ramosa (WO 2010/080619), P. thornea (WO 2010/080619), P. usgae BL1 (ATCC SD-5835; J. Nematol. 42(2): 87-90, 2010;/bid. 43(2), 101-109, 2011; e. g. Econem™ for control of Belonolaimus longicaudatus from Pasteuria BioScience now Syngenta sold by Harell's LLC, Florida, USA for use on turf for management of Belonolaimus longicaudatus), Penicillium bilaiae (also called P. bilaii) strains ATCC 18309 (=ATCC 74319), ATCC 20851 and/or ATCC 22348 (=ATCC 74318) originally isolated from soil in southern Alberta (Fertilizer Res. 39, 97-103, 1994; Can. J. Plant Sci. 78(1), 91-102, 1998; U.S. Pat. No. 5,026,417, WO 1995/017806; e. g. Jump Start®, Provide® from Novozymes Biologicals BioAg Group, Canada), P. bilaiae NRRL 50162 and NRRL 50169 (WO 2010/037228), Phlebiopsis gigantea (e. g. RotStop® from Verdera Oy, Finland), Pichia anomala WRL-076 (NRRL Y-30842; U.S. Pat. No. 8,206,972), potassium bicarbonate (e. g. Amicarb® from Stähler SA, Switzerland), potassium silicate (e. g. Sil-MATRIX™ from Certis LLC, USA), Pseudozyma flocculosa PF-A22 UL (e. g. Sporodex® L from Plant Products Co. Ltd., Canada), Pseudomonas sp. Proradix (DSM 13134; WO 2001/40441, e. g. PRORADIX from Sourcon Padena GmbH & Co. KG, Hechinger Str. 262, 72072 Tübingen, Germany), P. chloraphis MA 342 (Microbiology Monographs 18, 21-43, 2011; e. g. Cerall® or Cedemon® from BioAgri AB, Uppsala, Sweden or Intrachem Bio Deutschland GmbH & Co. KG, Bad Camberg, Germany), P. fluorescens (e.g. in Bio Cure-B from T. Stanes & Company Limited, India; or in Blight-End from Agri Naturals, Mumbai, India), P. fluorescens A506 (Phytopathol 97(2), 244-249, 2007; ATCC 31948; e. g. BlightBan® from NuFarm Americas, Inc., Morrisville, N.C., USA), P. fluorescens ATCC 13525 of biovar I=biotype A; originally isolated from pre-filter tanks in England (DSM 50090; registered for use in Canada), P. fluorescens CHA0 (Mol. Plant Microbe Interact. 5(1), 4-13, 1992), P. fluorescens CL 145A (J. Invertebr. Pathol. 113(1), 104-14, 2013; e. g. Zequanox® from Marrone BioInnovations, Davis, Calif., USA), P. fluorescens NCIB 12089 (EP 0210734 A!; Victus® from Mauri Laboratories, 9 Moorebank Ave., Moorebank, NSW 2170, Australia), P. fluorescens Pf-5 isolated from root surface of cotton (ATCC BAA-477), P. putida ATCC 202153 (EMBRAPA 63/88 4 B; WO 2004/0245865), Pythium oligandrum DV 74 (US 2013/0035230; ATCC 38472; e. g. Poyversum® from Remeslo SSRO, Biopreparaty, Czech Rep. and from Gowan, USA), Reynoutria sachalinensis extract (EP 0307510 B1; e. g. Regalia® SC from Marrone BioInnovations, Davis, Calif., USA or Milsana® from BioFa AG, Germany), Rhizobium leguminosarum bv. phaseoli (e. g. RHIZO-STICK from BASF Corp., USA), R. leguminosarum bv. phaseoli RG-B10 (USDA 9041; from Int. J. Syst. Bacteriol. 46(1), 240-244, 1996; Int. J. Syst. Evol. Microbiol. 50, 159-170, 2000; e. g. Nodulator® Dry Bean in Africa, HiStick NT Dry bean in US, and Nodulator® Dry Bean in Canada from BASF Corp., USA, or BASF Agricultural Specialties Ltd., Canada), R. l. bv. trifolii CB782 (Nodulaid® peat for Kenya white clover from BASF Agricultural Specialties Pty Ltd, Australia), R. l. bv. trifolii CC275e (Nodulaid® peat for NZ white clover from BASF Agricultural Specialties Pty Ltd, Australia), R. l. bv. trifolii CC283b (ICMP 4073b; Proc. New Zealand Grassland Assoc. 56, 101-105, 1994; Microbiol. 153, 3184-3195, 2007; Nodulaid® peat for Caucasian clover from BASF Agricultural Specialties Pty Ltd, Australia), R. l. bv. trifolii CC1099 (Inoculating Legumes: A Practical Guide, ed. Grain Research and Development Corporation, 2012, ISBN 978-1-921779-45-9; e. g. Nodulaid® peat for sainfoin from BASF Agricultural Specialties Pty Ltd, Australia), R. l. bv. trifolii RP113-7 (Appl. Environ. Microbiol. 44(5), 1096-1101, 1982; e. g. Dormal® from BASF Corp., USA), R. l. bv. trifolii TA1 (Appl. Environ. Microbiol. 49(1), 127-131, 1985; e. g. Nodulaid® peat for white clover from BASF Agricultural Specialties Pty Ltd, Australia), R. l. bv. trifolii strain WSM1325 isolated in 1993 from the Greek Island of Serifos (Stand. Genomic Sci. 2(3), 347-356, 2010; Inoculating Legumes: A Practical Guide, ed. Grain Research and Development Corporation, 2012, ISBN 978-1-921779-45-9; Nodulaid® peat for sub clover and Nodulator® granules for sub clover both from BASF Agricultural Specialties Pty Ltd, Australia, for a broad range of annual clovers of Mediterranean origin), R. l. bv. trifolii strain WSM2304 isolated from Trifolium polymorphum in Uruguay in 1998 (Stand. Genomic Sci. 2(1), 66-76, 2010), R. l. bv. viciae P1NP3Cst being a Streptomycin-resistant mutant of P1 NP3C isolated from pea root nodules in Breteniére, France (also referred to as 1435; New Phytol. 176, 680-690, 2007; ibid. 179(1), 224-235, 2008; e. g. Nodulator® PL Peat Granule from BASF Corp., USA; or Nodulator® XL PL from BASF Agricultural Specialties Ltd., Canada), R. l. bv. viciae RG-P2 also called P2 isolated from pea root nodules in Sakatchewan, Canada (e. g RhizUP peat for peas and lentils in Canada from BASF Agricultural Specialties Ltd., Canada), R. l. bv. viciae SU303 (e. g. Nodulaid® Group E from BASF Agricultural Specialties Pty Ltd, Australia), R. l. bv. viciae WSM1455 (e. g. Nodulaid® Group F from BASF Agricultural Specialties Pty Ltd, Australia), R. tropici CC511 (Agronomy, N.Z. 36, 4-35, 2006; e. g. Nodulaid® peat for common bean from BASF Agricultural Specialties Pty Ltd, Australia) R. tropici CIAT 899 isolated in Colombia (SEMIA 4077; Rev. Ci\textcircled{e}nc. Agron. 44(4) Fortaleza October/December 2013; e. g. Nitrafix® FEIJÃO peat for beans from BASF Agricultural Specialties Ltd., Brazil in mixture with strain SEMIA 4080), R. tropici H12 isolated in Planaltina, DF, Cerrados, Brazil (SEMIA 4088; Appl. Microbiol. Biotechnol. 93(5), 2035-49, 2012; e. g. Nitrafix® FEIJÃO from BASF Agricultural Specialties Ltd., Brazil), R. tropici PRF 81 isolated in Paraná, Brazil (SEMIA 4080; Soil Biology & Biochemistry 39, 867-876, 2007; BMC Microbiol. 12, 84, 2012; Nitrafix® FEIJÃO peat for beans from BASF Agricultural Specialties Ltd., Brazil in mixture with strain SEMIA 4077), Sinorhizobium meliloti RCR2011 also called 2011 or SU47 (MSDJ0848; Mol. Gen. Genomics 272, 1-17, 2004; e. g. Dormal® Alfalfa & Luzerne from BASF Corp., USA; Nitragin® Gold from Novozymes Biologicals BioAg Group, Canada), Sphaerodes mycoparasitica SMCD2220 also called SMCD2220-01 (IDAC 301008-01; WO 2011/022809), Spodoptera littoralis nucleopolyhedrovirus (SpliNPV) (e.g. in LITTOVIR from Adermatt Biocontrol, Switzerland), Steinernema carpocapsae (e. g. Millenium® from BASF Agricultural Specialities Limited, UK), S. feltiae (Nemashield® from BioWorks, Inc., USA; Nemasys® from BASF Agricultural Specialities Limited, UK), S. kraussei L137 (Nemasys® L from BASF Agricultural Specialities Limited, UK), Streptomyces galbus AQ6047 (NRRL 30232; WO 2012/135763; AgraQuest now Bayer CropScience LP, USA); S. galbus M1064 (NRRL 50334; WO 2012/135763; AgraQuest now Bayer CropScience LP, USA); S. griseoviridis K61 (Crop Protection 25, 468-475, 2006; e. g. Mycostop® from Verdera Oy, Espoo, Finland), S. lydicus WYEC 108 (U.S. Pat. No. 5,403,584; e. g. Actinovate® from Natural Industries, Inc., USA), S. violaceusniger YCED-9 (U.S. Pat. No. 5,968,503; e. g. DT-9® from Natural Industries, Inc., USA), Talaromyces flavus V117b isolated from soil (e. g. Protus® WG from Prophyta, Germany), Trichoderma asperellum SKT-1 isolated from the rhizosphere of Japanese lawngrass (FERM P-16510; J. Gen. Plant Pathol. 71(5), 351-356, 2005; e. g. Eco-Hope® from Kumiai Chemical Industry Co., Ltd., Japan), T. asperellum ICC 012 isolated from a soil in central Italy that was found to suppress plant disease (IMI 392716; e. g. Tenet WP, Remdier WP or Bioten WP from Isagro N.C., USA, Bio-Tam™ from AgraQuest, USA), T. asperellum TV1 formerly T. viride (MUCL 43093; e. g. T. viride TV1 from Agribiotec srl, Italy or Xedavir from Xeda Italia, Italy), T. atroviride LC52 (e. g. Sentinel® from Agrimm Technologies Ltd, NZ), T. atroviride CNCM I-1237 (e. g. Esquive® WG from Agrauxine S.A., France, e. g. against pruning wound diseases on vine and plant root pathogens), T. fertile JM41R (NRRL 50759; e. g. Trichoplus™ from BASF Agricultural Specialities (Pty) Ltd., South Africa), T. gamsii ICC 080 (IMI 392151; e. g. Tenet WP, Remdier WP, Bioten WP from Isagro N.C., USA, Bio-Tam™ from AgraQuest, USA), T. harzianum T-22 also called KRL-AG2 (ATCC 20847; BioControl 57, 687-696, 2012; e. g. Plantshield® from BioWorks Inc., USA or SabrEx™ from Advanced Biological Marketing Inc., Van Wert, Ohio, USA), T. harzianum T-35 and T-315 (ATCC 20691; EP 0133878 B1; e. g. Root Pro® from Mycontrol Ltd., Israel), T. harzianum T-39 (CNCM I-952; EP 0466133 B2; e. g. Trichodex® or Trichoderma 2000® from Mycontrol Ltd., Israel and Makhteshim Ltd., Israel), mixture of T. harzianum and T. viride (e. g. Trichopel® from Agrimm Technologies Ltd, NZ), mixture of T. harzianum ICC012 and T. viride ICC080 (e. g. Remdier® WP from Isagro Ricerca, Italy), T. polysporum IMI 206039 (ATCC 20476; e. g. Binab® from BINAB Bio-Innovation AB, Sweden in mixture with T. atroviride IMI 206040), T. stromaticum (e. g. Tricovab® from C.E.P.L.A.C., Brazil), T. virens GI-3 also called G1-3 or GL-3 (CA 2471555 A1; ATCC 58678; e.g. QuickRoots™ from TJ Technologies, Watertown, S. Dak., USA in mixture with B. amyloliquefaciens TJ1000), T. virens GL-21 also called G1-21 isolated from a sclerotium of Sclerotinia minor (U.S. Pat. No. 7,429,477; e. g. Soilguard® 12G from Certis LLC, USA; EPA Registration Number: 70051-3 and EPA Establishment Number: 067250-IL-001), T. virens G-41 also called 041, #41X or ABM 127 isolated from soil samples taken from Aphanomyces-suppressive bean fields in Livingston County, New York (ATCC 20906; U.S. Pat. No. 4,996,157; e. g. Rootshield® PLUS from BioWorks, Inc., USA), T. viride (J. Biological Control 23(1), 31-36, 2009; e. g. Trieco® from Ecosense Labs. (India) Pvt. Ltd., India; or Bio-Cure® F from T. Stanes & Co. Ltd., India), and Ulocladium oudemansii HRU3 (Agronomy 3, 632-647, 2013; e. g. Botry-Zen® from Botry-Zen Ltd, NZ).

Strains can be obtained from culture collections and deposition centers (listed by their acronym=strain prefix here: http://www.wfcc.info/ccinfo/collection/by_acronym/) such as strains with prefices AGAL or NMI from: National Measurement Institute, 1/153 Bertie Street, Port Melbourne, Victoria, Australia 3207; ATCC: American Type Culture Collection, 10801 University Blvd., Manassas, Va. 20110-2209, USA; BR: Embrapa Agrobiology Diazothrophic Microbial Culture Collection, P.O. Box 74.505, Seropedica, Rio de Janeiro, 23.851-970, Brazil; CABI or IMI: CABI Europe—International Mycological Institute, Bakeham Lane, Egham, Surrey, TW20 9TYNRRL, UK; CB: The CB Rhizobium Collection, School of Environment and Agriculture, University of Western Sydney, Hawkesbury, Locked Bag 1797, South Penrith Distribution Centre, NSW 1797, Australia; CBS: Centraalbureau voor Schimmelcultures, Fungal Biodiversity Centre, Uppsalaan 8, PO Box 85167, 3508 AD Utrecht, Netherlands; CC: Division of Plant Industry, CSIRO, Canberra, Australia; CNCM: Collection Nationale de Cultures de Microorganismes, Institute Pasteur, 25 rue du Docteur Roux, F-75724 PARIS Cedex 15; CPAC: Embrapa-Cerrados, CX. Postal 08223, Planaltina, DF, 73301-970, Brazil; DSM: Leibniz-Institut DSMZ-Deutsche Sammlung von Mikroorganismen and Zellkulturen GmbH, Inhoffenstraβe 7 B, 38124 Braunschweig, Germany; IDAC: International Depositary Authority of Canada Collection, Canada; ICMP: International Collection of Micro-organisms from Plants, Landcare Research, Private Bag 92170, Auckland Mail Centre, Auckland 1142, New Zealand; IITA: IITA, PMB 5320, Ibadan, Nigeria; INTA: Agriculture Collection Laboratory of the Instituto de Microbiologia y Zoologia Agricola (IMYZA), Instituto Nacional de Tecnologl'a Agropecuaria (INTA), Castelar, Argentina; MSDJ: Laboratoire de Microbiologie des Sols, INRA, Dijon, France; MUCL: Mycothèque de l'Université catholique de Louvain, Croix du Sud 2, box L7.05.06, 1348 Louvain-la-Neuve, Belgium; NCIMB or NICB: The National Collections of Industrial and Marine Bacteria Ltd., Torry Research Station, P.O. Box 31, 135 Abbey Road, Aberdeen, AB9 8DG, Scotland; Nitragin: Nitragin strain collection, The Nitragin Company, Milwaukee, Wis., USA, NRRL or ARSEF (collection of entomopathogenic fungi): ARS Culture Collection of the National Center for Agricultural Utilization Research, Agricultural Research Service, U.S. Department of Agriculture, 1815 North University Street, Peoria, Ill. 61604, USA; NZP: Department of Scientific and Industrial Research Culture Collection, Applied Biochemistry Division, Palmerston North, New Zealand; PPRI: ARC-Plant Protection Research Institute, Private Bag X134, Queenswood Pretoria, Gauteng, 0121, South Africa; SEMIA: FEPAGRO-Fundação Estadual de Pesquisa Agropecuária, Rua Gonçalves Dias, 570, Bairro Menino Deus, Porto Alegre/RS, Brazil; SRDI: SARDI, Adelaide, South Australia; USDA: U.S. Department of Agriculture, Agricultural Research Service, Soybean and Alfalfa Research Laboratory, BARC-West, 10300 Baltimore Boulevard, Building 011, Beltsville, Md. 20705, USA (Beltsville Rhiz. Cult. Catalog: http://pdf.usaid.gov/pdf_docs/PNAAW891.pdf); and WSM: Murdoch University, Perth, Western Australia. Further strains may be found at: http://gcm.wfcc.info/; http://www.landcareresearch.co.nz/resources/collections/icmp.

Jasmonic acid, its salts (jasmonates) or derivatives include without limitation potassium, sodium, lithium, ammonium, dimethylammonium, isopropylammonium, diolammonium and diethtriethanolammonium jasmonate; and also jasmonic acid methyl ester, jasmonic acid amide, jasmonic acid methylamide, jasmonic acid-L-amino acid (amide-linked) conjugates (e. g. conjugates with L-isoleucine, L-valine, L-leucine, or L-phenylalanine), 12-oxo-phytodienoic acid, coronatine, coronalon, coronafacoyl-L-serine, coronafacoyl-L-threonine, methyl esters of 1-oxo-indanoyl-isoleucine, methyl esters of 1-oxo-indanoyl-leucine, cis-jasmone, linoleic acid or derivatives thereof, and combinations of any of the above.

Humates are humic and fulvic acids extracted from a form of lignite coal and clay, known as leonardite. Humic acids are organic acids that occur in humus and other organically derived materials such as peat and certain soft coal. They have been shown to increase fertilizer efficiency in phosphate and micro-nutrient uptake by plants as well as aiding in the development of plant root systems.

According to one embodiment of the inventive mixtures, the at least one pesticide II is selected from the groups L1) to L6):
L1) Microbial pesticides with fungicidal, bactericidal, viricidal and/or plant defense activator activity: Ampelomyces quisqualis M-10 (L.1.1), Aspergillus flavus NRRL 21882 (L1.2), Aureobasidium pullulans DSM 14940 (L1.3), A. pullulans DSM 14941 (L.1.4), Bacillus altitudinis 41 KF2b (L.1.5), Bacillus amyloliquefaciens AP-136 (L.1.6), B. amyloliquefaciens AP-188 (L.1.7), B. amyloliquefaciens AP-218 (L.1.8), B. amyloliquefaciens AP-219 (L.1.9), B. amyloliquefaciens AP-295 (L.1.10), B. amyloliquefaciens IN937a (L.1.11), B. amyloliquefaciens IT-45 (L.1.12), B. amyloliquefaciens ssp. plantarum D747 (L.1.13), B. amyloliquefaciens ssp. plantarum FZB24 (L.1.14), B. amyloliquefaciens ssp. plantarum FZB42 (L.1.15), B. amyloliquefaciens ssp. plantarum GB03 (L.1.16), B. amyloliquefaciens ssp. plantarum MBI600 (NRRL B-50595) (L.1.17), B. amyloliquefaciens ssp. plantarum QST-713 (L.1.18), B. amyloliquefaciens ssp. plantarum TJ1000 (L.1.19), B. mojavensis AP-209 (L.1.20), B. mycoides AQ726 (L.1.21), B. mycoides strain J (L.1.22), B. pumilus INR-7 (L.1.23), B. pumilus KFP9F (L.1.24), B. pumilus QST 2808 (L.1.25), B. pumilus GHA 180 (L.1.26), B. simplex ABU 288 (L.1.27), B. solisalsi AP-217 (L.1.28), B. subtilis CX-9060 (L.1.29), B. subtilis FB17 (L.1.30), B. subtilis GB07 (L.1.31), Candida oleophila I-82 (L.1.32), C. oleophila O (L.1.33), C. saitoana (L.1.34), Clavibacter michiganensis S (bacteriophages) (L.1.35), Coniothyrium minitans CON/M/91-08 (L.1.36), Cryphonectria parasitica (L.1.37), Cryptococcus albidus (L.1.38), Dilophosphora alopecuri (L.1.39), Fusarium oxysporum (L.1.40), Clonostachys rosea f. catenulata J1446 (L.1.41), Gliocladium roseum 321U (L.1.42), Metschnikowia fructicola NRRL Y-30752 (L.1.43), Microdochium dimerum (L.1.44), Microsphaeropsis ochracea P130A (L.1.45), Muscodor albus QST 20799 (L.1.46), Muscodor albus SA-13 (L.1.47), Paenibacillus alvei NAS6G6 (L.1.48), Paenibacillus polymyxa PKB1 (L.1.49), Pantoea agglomerans E325 (L.1.90), Pantoea vagans C9-1 (L.1.50), Penicillium bilaiae ATCC 22348 (L.1.51), P. bilaiae ATCC 20851 (L.1.52), Penicillium bilaiae ATCC 18309 (L.1.53), Phlebiopsis gigantea (L.1.54), Pichia anomala WRL-76 (L.1.55), Pseudomonas sp. Proradix (L.1.56), Pseudomonas chloraphis MA 342 (L.1.57), P. fluorescens A506 (L.1.58), P. fluorescens CL 145A (L.1.91), P. fluorescens NCIB 12089 (L.1.92), P. fluorescens Pf-5 (L.1.93), P. fluorescens WCS 374 (L.1.94), P. fluorescens ATCC 13525 (L.1.95), P. fluorescens CHA0 (L.1.96), P. putida ATCC 202153 (L.1.97), Pseudozyma flocculosa PF-A22 UL (L.1.59), Pythium oligandrum DV 74 (L.1.60), Sphaerodes mycoparasitica SMCD2220 (L.1.61), Streptomyces griseoviridis K61 (L.1.62), S. lydicus WYEC 108 (L.1.63), S. violaceusniger XL-2 (L.1.64), S. violaceusniger YCED-9 (L.1.65), Talaromyces flavus V117b (L.1.66), Trichoderma asperellum T34 (L.1.67), T. asperellum SKT-1 (L.1.68), T. asperellum ICC 012 (L.1.69), T. atroviride LC52 (L.1.70), T. atroviride CNCM I-1237 (L.1.71), T. fertile JM41R (L.1.72), T. gamsii ICC 080 (L.1.73), T. harmatum TH 382 (L.1.74), T. harzianum T-35 (L.1.75), T. harzianum T-22 (L.1.76), T. harzianum T-39 (L.1.77); mixture of T. harzianum ICC012 and T. viride ICC080 (L.1.78); T. polysporum (L.1.79); T. stromaticum (L.1.80), T. virens GI-3 (L.1.81), T. virens G-41 (L.1.82), T. virens GL-21 (L.1.83), T. virens G-41 (L.1.84), T. viride TV1 (L.1.85), Typhula phacorrhiza 94671 (L.1.86), Ulocladium oudemansii HRU3 (L.1.87), Verticillium dahlia (L.1.88), zucchini yellow mosaic virus (avirulent strain) (L.1.89);
L2) Biochemical pesticides with fungicidal, bactericidal, viricidal and/or plant defense activator activity: chitosan (hydrolysate) (L.2.1), harpin protein (L.2.2), laminarin (L.2.3), Menhaden fish oil (L.2.4), natamycin (L.2.5), Plum pox virus coat protein (L.2.6), potassium bicarbonate (L.2.7), Reynoutria sachalinensis extract (L.2.8), salicylic acid (L.2.9), potassium or sodium bicarbonate (L.2.10), tea tree oil (L.2.11);
L3) Microbial pesticides with insecticidal, acaricidal, molluscidal and/or nematicidal activity: Agrobacterium radiobacter K1026 (L.3.1), A. radiobacter K84 (L.3.2), Bacillus firmus I-1582 (L.3.3); B. thuringiensis ssp. aizawai strains: ABTS-1857 (L.3.4), SAN 401 I (L.3.5), ABG-6305 (L.3.6) and ABG-6346 (L.3.7); B. t. ssp. israelensis AM65-52 (L.3.8), B. t. ssp. israelensis SUM-6218 (L.3.9), B. t. ssp. galleriae SDS-502 (L.3.10), B. t. ssp. kurstaki EG 2348 (L.3.11), B. t. ssp. kurstaki SB4 (L.3.12), B. t. ssp. kurstaki ABTS-351 (HD-1) (L.3.13), Beauveria bassiana ATCC 74040 (L.3.14), B. bassiana GHA (L.3.15), B. bassiana H123 (L.3.16), B. bassiana DSM 12256 (L.3.17), B. bassiana PPRI 5339 (L.3.18), B. brongniartii (L.3.19), Burkholderia sp. A396 (L.3.20), Chromobacterium subtsugae PRAA4-1 (L.3.21), Cydia pomonella granulosis virus V22 (L.3.22), Cydia pomonella granulosis virus V1 (L.3.23), Cryptophlebia leucotreta granulovirus (CrleGV) (L.3.57), Flavobacterium sp. H492 (L.3.60), Helicoverpa armigera nucleopolyhedrovirus (HearNPV) (L.3.58), Isaria fumosorosea Apopka-97 (L.3.24), Lecanicillium longisporum KV42 (L.3.25), L. longisporum KV71 (L.3.26), L. muscarium KV01 (L.3.27), Metarhizium anisopliae FI-985 (L.3.28), M. anisopliae FI-1045 (L.3.29), M. anisopliae F52 (L.3.30), M. anisopliae ICIPE 69 (L.3.31), M. anisopliae var. acridum IMI330189 (L.3.32); Nomuraea rileyi strains: SA86101 (L.3.33), GU87401 (L.3.34), SR86151 (L.3.35), CG128 (L.3.36) and VA9101 (L.3.37); Paecilomyces fumosoroseus FE 9901 (L.3.38), P. lilacinus 251 (L.3.39), P. lilacinus DSM 15169 (L.3.40), P. lilacinus BCP2 (L.3.41), Paenibacillus popilliae Dutky-1940 (NRRL B-2309=ATCC 14706) (L.3.42), P. popilliae Dutky 1 (L.3.43), P. popilliae KLN 3 (L.3.56), Pasteuria sp. Ph3 (L.3.44), Pasteuria sp. ATCC PTA-9643 (L.3.45), Pasteuria sp. ATCC SD-5832 (L.3.46), P. nishizawae Pn1 (L.3.46), P. penetrans (L.3.47), P. ramosa (L.3.48), P. sp. Pr-3 (L.3.49), P. thornea (L.3.50), P. usgae (L.3.51), Pseudomonas fluorescens CL 145A (L.3.52), Spodoptera littoralis nucleopolyhedrovirus (SpliNPV) (L.3.59), Steinernema carpocapsae (L.3.53), S. feltiae (L.3.54), S. kraussei L137 (L.3.55);
L4) Biochemical pesticides with insecticidal, acaricidal, molluscidal, pheromone and/or nematicidal activity: L-carvone (L.4.1), citral (L.4.2), (E,Z)-7,9-dodecadien-1-yl acetate (L.4.3), ethyl formate (L.4.4), (E,Z)-2,4-ethyl decadienoate (pear ester) (L.4.5), (Z,Z,E)-7,11,13-hexadecatrienal (L.4.6), heptyl butyrate (L.4.7), isopropyl myristate (L.4.8), cis-jasmone (L.4.9), lavanulyl senecioate (L.4.10), 2-methyl 1-butanol (L.4.11), methyl eugenol (L.4.12), methyl jasmonate (L.4.13), (E,Z)-2,13-octadecadien-1-ol (L.4.14), (E,Z)-2,13-octadecadien-1-ol acetate (L.4.15), (E,Z)-3,13-octadecadien-1-ol (L.4.16), R-1-octen-3-ol (L.4.17), pentatermanone (L.4.18), potassium silicate (L.4.19), sorbitol actanoate (L.4.20), (E,Z,Z)-3,8,11-tetradecatrienyl acetate (L.4.21), (Z,E)-9,12-tetradecadien-1-yl acetate (L.4.22), Z-7-tetradecen-2-one (L.4.23), Z-9-tetradecen-1-yl acetate (L.4.24), Z-11-tetradecenal (L.4.25), Z-11-tetradecen-1-ol (L.4.26), Acacia negra extract (L.4.27), extract of grapefruit seeds and pulp (L.4.28), extract of Chenopodium ambrosiodes (L.4.29), Catnip oil (L.4.30), Neem oil (L.4.31), Quillay extract (L.4.32), Tagetes oil (L.4.33);
L5) Microbial pesticides with plant stress reducing, plant growth regulator, plant growth promoting and/or yield enhancing activity: Azosprillum amazonense BR 11140 (SpY2) (L.5.1), A. brasilense Ab-V5 (L.5.74), A. brasilense Ab-V6 (L.5.75), A. brasilense AZ39 (L.5.2), A. brasilense XOH (L.5.3), A. brasilense Sp245 (BR 11005) (L.5.4), A. brasilense BR 11002 (L.5.5), A. lipoferum BR 11646 (Sp31) (L.5.6), A. irakense (L.5.7), A. halopraeferens (L.5.8), Bradyrhizobium sp. PNL01 (L.5.9), B. sp. (Arachis) CB1015 (L.5.10), B. sp. (Arachis) USDA 3446 (L.5.11), B. sp. (Arachis) SEMIA 6144 (L.5.12), B. sp. (Arachis) SEMIA 6462 (L.5.13), B. sp. (Arachis) SEMIA 6464 (L.5.14), B. sp. (Vigna) (L.5.15), B. elkanii SEMIA 587 (L.5.16), B. elkanii SEMIA 5019 (L.5.17), B. elkanii U-1301 (L.5.18), B. elkanii U-1302 (L.5.19), B. elkanii USDA 74 (L.5.20), B. elkanii USDA 76 (L.5.21), B. elkanii USDA 94 (L.5.22), B. elkanii USDA 3254 (L.5.23), B. japonicum 532c (L.5.24), B. japonicum CPAC 15 (L.5.25), B. japonicum E-109 (L.5.26), B. japonicum G49 (L.5.27), B. japonicum TA-11 (L.5.28), B. japonicum USDA 3 (L.5.29), B. japonicum USDA 31 (L.5.30), B. japonicum USDA 76 (L.5.31), B. japonicum USDA 110 (L.5.32), B. japonicum USDA 121 (L.5.33), B. japonicum USDA 123 (L.5.34), B. japonicum USDA 136 (L.5.35), B. japonicum SEMIA 566 (L.5.36), B. japonicum SEMIA 5079 (L.5.37), B. japonicum SEMIA 5080 (L.5.38), B. japonicum WB74 (L.5.39), B. liaoningense (L.5.40), B. lupini LL13 (L.5.41), B. lupini WU425 (L.5.42), B. lupini WSM471 (L.5.43), B. lupini WSM4024 (L.5.44), Glomus intraradices RTI-801 (L.5.45), Mesorhizobium sp. WSM1271 (L.5.46), M. sp. WSM1497 (L.5.47), M. ciceri CC1192 (L.5.48), M. huakii (L.5.49), M. loti CC829 (L.5.50), M. loti SU343 (L.5.51), Rhizobium leguminosarum bv. phaseoli RG-B10 (L.5.52), R. l. bv. trifolii RP113-7 (L.5.53), R. l. bv. trifolii 095 (L.5.57), R. l. bv. trifolii TA1 (L.5.58), R. l. bv. trifolii CC283b (L.5.59), R. l. bv. trifolii CC275e (L.5.60), R. l. bv. trifolii CB782 (L.5.61), R. l. bv. trifolii CC1099 (L.5.62), R. l. bv. trifolii WSM1325 (L.5.63), R. l. bv. viciae SU303 (L.5.64), R. l. bv. viciae WSM1455 (L.5.65), R. l. bv. viciae P1NP3Cst (L.5.66), R. l. bv. viciae RG-P2 (L.5.67), R. tropici PRF 81 (L.5.68), R. tropici SEMIA 4077 (L.5.69), R. tropici CC511 (L.5.70), Sinorhizobium meliloti RCR2011 (L.5.71), S. meliloti NRG185 (L.5.72), S. meliloti RRI128 (L.5.73);
L6) Biochemical pesticides with plant stress reducing, plant growth regulator and/or plant yield enhancing activity: abscisic acid (L.6.1), aluminium silicate (kaolin) (L.6.2), 3-decen-2-one (L.6.3), formononectin (L.6.4), genistein (L.6.5), hesperetin (L.6.6), homobrassinolide (L.6.7), humates (L.6.8), methyl jasmonate (L.6.9), cis-jasmone (L.6.10), lysophosphatidyl ethanlamine (L.6.11), naringenin (L.6.12), polymeric polyhydroxy acid (L.6.13), salicylic acid (L.6.14), Ascophyllum nodosum (Norwegian kelp, Brown kelp) extract (L.6.15) and Ecklonia maxima (kelp) extract (L.6.16).

The present invention furthermore relates to agrochemical compositions comprising a mixture of compound I (component 1) and at least one biopesticide selected from the group L) (component 2), in particular at least one further fungicidal biopesticide selected from the groups L1) and L2), as described above, and if desired at least one suitable auxiliary.

Preference is also given to mixtures comprising as pesticide II (component 2) a biopesticide from group L1), preferably selected from Bacillus amyloliquefaciens herein even more preferably from strains AP-136, AP-188, AP-218, AP-219, AP-295, IN937a, IT-45; B. amyloliquefaciens ssp. plantarum (formerly called B. subtilis or B. subtilis spp. amyloliquefaciens) herein even more preferably from strains MBI600, D747, FZB254, FZB42, GB03, QST-713 and TJ1000; B. mojavensis AP-209; B. pumilus herein even more preferably from strains GHA 180, INR-7, KFP9F and QST 2808; B. simplex herein more preferably strain ABU 288; B. solisalsi herein more preferably strain AP-217; B. subtilis herein even more preferably selected from strains CX-9060, FB17 and GB07; Muscodor albus herein more preferably strains QST 20799 and SA-13; Paenibacillus alvei herein more preferably strain NAS6G6, Paenibacillus polymyxa herein more preferably strain PKB1, Penicillium bilaiae herein more preferably strains ATCC 22348, ATCC 20581 and ATCC 18309; Pseudomonas fluorescens herein more preferably strain A506; Sphaerodes mycoparasitica herein more preferably strain SMCD2220; Trichoderma fertile herein more preferably strain JM41R; Trichoderma harzianum herein more preferably strain T-22; Trichoderma virens herein more preferably strains GI-3 and G-41.

Preference is also given to mixtures comprising as pesticide II (component 2) a biopesticide from group L1), even more preferably selected from even more preferably from B. amyloliquefaciens AP-188, B. amyloliquefaciens ssp. plantarum MBI600, B. amyloliquefaciens ssp. plantarum QST-713, B. pumilus INR-7, B. pumilus QST 2808, B. simplex ABU 288, B. subtilis FB17, Paenibacillus alvei NAS6G6 and Trichoderma fertile JM41R.

According to one embodiment of the inventive mixtures, the at least one pesticide II is Bacillus amyloliquefaciens ssp. plantarum MBI600. These mixtures are particularly suitable in soybean. According to another embodiment of the inventive mixtures, the at least one pesticide II is B. pumilus INR-7. These mixtures are particularly suitable in soybean and corn.

According to a further embodiment, the at least one pesticide II is Bacillus simplex, preferably B. simplex ABU 288. These mixtures are particularly suitable in soybean and corn.

According to a further embodiment, the at least one pesticide II is Bacillus subtilis, preferably B. subtilis strain FB17.

According to one embodiment of the inventive mixtures, the at least one pesticide II is selected from Bacillus amyloliquefaciens AP-136, B. amyloliquefaciens AP-188, B. amyloliquefaciens AP-218, B. amyloliquefaciens AP-219, B. amyloliquefaciens AP-295, B. amyloliquefaciens spp. plantarum FZB24, B. amyloliquefaciens ssp. plantarum FZB42, B. amyloliquefaciens ssp. plantarum TJ1000, B. amyloliquefaciens ssp. plantarum D747, B. amyloliquefaciens ssp. plantarum MBI600, B. amyloliquefaciens spp. plantarum GB03, B. amyloliquefaciens spp. plantarum QST-713, B. mojavensis AP-209, B. pumilus GB34, B. pumilus INR-7, B. pumilus KFP9F, B. pumilus QST 2808, B. pumilus GHA 180, B. simplex ABU 288, B. solisalsi AP-217, B. subtilis CX-9060, B. subtilis FB17 and B. subtilis GB07. These mixtures are particularly suitable in soybean and corn, in particular for seed treatment.

According to a further embodiment, the at least one pesticide II is selected from Streptomyces spp., preferably from S. griseoviridis, S. lydicus and S. violaceusniger, in particular from strains S. griseoviridis K61, S. lydicus WYEC 108, S. violaceusniger XL-2 and S. violaceusniger YCED-9.

According to one embodiment of the inventive mixtures, the at least one pesticide II is selected from the following fungi Coniothyrium minitans CON/M/91-08, Trichoderma fertile JM41R, T. harzianum T-22, T. virens GI-3, T. virens GL-21, T. virens G-41. These mixtures are particularly suitable for seed and/or soil treatment.

The present invention also relates to mixtures wherein the at least one pesticide II is selected from the following yeasts and fungi: Ampelomyces quisqualls, in particular strain M-10; Aureobasidium pullulans, in particular blastospores of strain DSM14940 or blastospores of strain DSM 14941 or mixtures thereof; Candida oleophila, in particular strains I-182 and O; Coniothyrium minitans, in particular strain CON/M/91-8; Dilophosphora alopecuri which reduces annual ryegrass toxicity (ARGT), a disease of livestock resulting from the ingestion of annual ryegrass seed-heads that have been infected by the toxin producing bacterium Rathayibacter toxicus, Clonostachys rosea f. catenulata, in particular strain J1446; Metschnikovia fructicola, in particular strain 277, Microsphaeropsis ochracea, in particular strain P130A for control of apple scab; Muscodor albus, in particular strain QST 20799, Pichia anomala, in particular strain WRL-076, Pseudozyma flocculosa, in particular strain PF-A22 UL; Pythium oligandrum, in particular strain DV74.

According to a further embodiment, the at least one pesticide II is selected from Pseudomonas spp., preferably selected from P. chloraphis herein more preferably strain MA 342 and Pseudomonas sp. DSM 13134; P. fluorescens herein more preferably selected from strains A506, WCS 374 and Pf-5; and P. putida herein more preferably strain ATCC 202153.

The present invention also relates to mixtures wherein the at least one pesticide II is selected from the fungal genus Trichoderma, preferably from the strains T. asperellum T34, T. asperellum SKT-1, T. asperellum ICC 012, T. asperellum TV1, T. atroviride LC52, T. atroviride CNCM I-1237, T. fertile JM41R, T. gamsii ICC 080, T. harmatum TH 382, T. harzianum T-22, T. harzianum T-35, T. harzianum T-39, T. harzianum T-315; mixture of T. harzianum ICC012 and T. gamsii ICC080; mixture of T. polysporum and T. harzianum; T. stromaticum, T. virens GI-3, T. virens GL-21, T. virens G-41 and; in particular T. fertile JM41R.

The present invention also relates to mixtures wherein the at least one pesticide II is selected from the fungal species Muscodor albus preferably from the strains SA-13 and QST 20799, which are particularly suitable for soil and seed treatment against soil-borne pathogens and/or nematodes.

Preference is also given to mixtures comprising as pesticide II (component 2) a biopesticide from group L2), preferably selected from chitosan (hydrolysate), methyl-jasmonate, cis-jasmone, laminarin, Reynoutria sachalinensis extract and tea tree oil; even more preferable from methyl jasmonate, cis-jasmone and laminarin.

Preference is also given to mixtures comprising as pesticide II (component 2) a biopesticide from group L3), preferably selected from Agrobacterium radiobacter herein preferably strain K1026, Bacillus firmus herein preferably strain I-1582, Bacillus thuringiensis ssp. kurstaki herein preferably strain SB4, Beauveria bassiana herein preferably selected from strains GHA, H123, DSM 12256 and PPRI 5339; Burkholderia sp. and herein preferably strain A396, Metarhizium anisopliae var. acridum herein preferably strain IMI 330189, M. anisopliae herein preferably selected from strains FI-985, FI-1045, F52 and ICIPE 69; Paecilomyces lilacinus herein preferably selected from strains 251, DSM 15169 and BCP2, Paenibacillus popilliae herein preferably selected from strains Dutky-1940, KLN 3 and Dutky 1; Pasteuria nishazawa and herein preferably strain Pn1.

Preference is also given to mixtures comprising as pesticide II (component 2) a biopesticide from group L3), even more preferably from Bacillus thuringiensis ssp. kurstaki SB4, B. bassiana DSM 12256, B. bassiana PPRI 5339, Metarhizium anisopliae var. acridum IMI 330189, M. anisopliae FI-985, M. anisopliae FI-1045, Paecilomyces lilacinus DSM 15169, P. lilacinus BCP2, P. lilacinus 251, Paenibacillus popilliae Dutky-1940, P. popilliae KLN 3 and P. popilliae Dutky 1.

According to a further embodiment, the at least one pesticide II is Beauveria brongniartii.

According to a further embodiment, the at least one pesticide II is Metarhizium anisopliae or M. anisopliae var. acridum, preferably selected from M. anisopiiae FI-1045, M. anispliae F52, M. anispliae var. acridum strains FI-985 and IMI 330189; in particular strain IMI 330189. These mixtures are particularly suitable for control of arthropod pests in soybean and corn.

According to a further embodiment, the at least one pesticide II is Lecanicillium sp., preferably selected from Lecanicillium longisporum KV42, L. longisporum KV71 and L. muscarium KV01.

According to a further embodiment, the at least one pesticide II is Paecilomyces fumoso-roseus, preferably strain FE 9901 especially for white fly control.

According to a further embodiment, the at least one pesticide II is selected from Nomuraea rileyi, preferably strains SA86101, GU87401, SR86151, CG128 and VA9101; and P. lilacinus, preferably strains 251, DSM 15169 or BCP2, in particular BCP2, which strains especially control the growth of plant-pathogenic nematodes.

According to a further embodiment, the at least one pesticide II is Bacillus firmus, preferably spores of strain CNCM I-1582, preferably useful for seed treatment of soybean and corn against nematodes and insects.

According to a further embodiment, the at least one pesticide II is Bacillus cereus, preferably spores of CNCM I-1562, preferably useful for seed treatment of soybean and corn against nematodes and insects.

According to a further embodiment, the at least one pesticide II is a mixture of spores of B. firmus and B. cereus, preferably mixtures spores of above mentioned strains CNCM I-1582 and CNCM I-1562, preferably useful for seed treatment of soybean and corn against nematodes and insects.

According to a further embodiment, the at least one pesticide II is selected from Bacillus t. ssp. kurstaki preferably from strains EG 2348, SB4 and ABTS-351 (HD-1), in particular B. t. ssp. kurstaki SB4. These strains are used for control of lepidopteran larvae, but without noctuidae.

According to one embodiment of the inventive mixtures, the at least one pesticide II is selected from Bacillus firmus CNCM I-1582, Paecilomyces lilcinus 251, Pasteuria nishizawa Pn1 and Burkholderia sp. A396 having nematicidal, acaricidal and/or insecticidal activity. These mixtures are particularly suitable in soybean and corn, in particular for seed treatment.

Preference is also given to mixtures comprising as pesticide II (component 2) a biopesticide from group L4), preferably selected from methyl jasmonate, Acacia negra extract, extract of grapefruit seeds and pulp, Catnip oil, Neem oil, Quillay extract and Tagetes oil, in particular methyl jasmonate or water-based Quillay extract.

Preference is also given to mixtures comprising as pesticide II (component 2) a biopesticide from group L5), preferably selected from Azospirillum amazonense, A. brasilense, A. lipoferum, A. irakense, A. halopraeferens, Bradyrhizobium sp. (Arachis), Bradyrhizobium sp. (Vigna), B. elkanii, B. japonicum; Paenibacillus alvei, Penicillium bilaiae, Rhizobium leguminosarum bv. phaseoli, R. l. bv. trifolii, R. l. bv. viciae, and Sinorhizobium meliloti.

Preference is also given to mixtures comprising as pesticide II (component 2) a biopesticide from group L5) selected from Azospirillum amazonense SpY2, A. brasilense XOH, A. brasilense Sp245, A. brasilense Cd, A. brasilense Ab-V5, A. brasilense Ab-V6, A. lipoferum Sp31, Bradyrhizobium sp. (Vigna) PNL1, B. elkanii SEMIA 587, B. elkanii SEMIA 5019, B. japonicum SEMIA 5079, B. japonicum SEMIA 5080, B. japonicum TA-11, B. japonicum 532c, Paenibacillus alvei NAS6G6, Peniciillium bilaiae strains ATCC 18309, ATCC 20851 and ATCC 22348; Rhizobium leguminosarum bv. phaseoli RG-B10, R. l. bv. viciae P1NP3Cst, R. l. bv. viciae R^{G}-P2, R. l. bv. trifolii RP113-7, R. l. bv. viciae SU303, R. l. bv. viciae WSM1455, R. tropici SEMIA 4077, R. tropici PRF 81 and Sinorhizobium meliloti, even more preferably selected from Azospirillum brasilense Sp245, Bradyrhizobium sp. (Vigna) PNL1, B B. elkanii SEMIA 587, B. elkanii SEMIA 5019, B. japonicum SEMIA 5079, B. japonicum SEMIA 5080, B. japonicum TA-11 and B. japonicum 532c.

The present invention also relates to mixtures, wherein the at least one pesticide II is selected from Azospirillum amazonense, A. brasilense, A. lipoferum, A. irakense and A. halopraeferens, more preferably from A. brasilense, in particular selected from A. brasilense strains Sp245 and AZ39 which are both commercially used in Brazil and are obtainable from EMBRAPA-Agribiologia, Brazil, and strains Ab-V5 and Ab-V6; in particular mixtures of these strains Ab-V5 and Ab-V6. These mixtures are particularly suitable in soybean, especially as seed treatment.

The present invention also relates to mixtures wherein the at least one pesticide II is selected from A. amazonense, A. brasiliense, A. lipoferum, A. irakense and A. halopraeferens, more preferably A. brasilense, and further comprises a pesticide III, wherein pesticide III is selected from jasmonic acid, its salts and derivatives thereof, preferably methyl-jasmonate or cis-jasmone.

According to another embodiment of the inventive mixtures, Bradyrhizobium spp. (meaning any Bradyrhizobium species and/or strain) as pesticide II is B. japonicum. These mixtures are particularly suitable in soybean. Certain B. japonicum strains have been re-classified as a novel species B. elkanii, e. g. strain USDA 76 (Can. J. Microbiol. 38, 501-505, 1992). Bradyrhizobium spp. are cultivated using media and fermentation techniques known in the art, e. g. in yeast extract-mannitol broth (YEM) at 27° C. for about 5 days.

The present invention also relates to mixtures, wherein the at least one pesticide II is selected from Bradyrhizobium spp., even more preferably from B. sp. (Arachis), B. elkanii, B. japonicum, B. liaoningense and B. lupini, and further comprises a pesticide III (component 3), wherein pesticide III is selected from jasmonic acid, its salts and derivatives thereof, preferably methyl-jasmonate or cis-jasmone.

Preferably, B. japonicum is selected from strains E-109, SEMIA 5079, SEMIA 5080, TA-11 and 532c. According to a further embodiment, mixtures of B. japonicum strains TA-11 and 532c or B. japonicum strains SEMIA 5079 and 5080 are used. The strains having a prefix SEMIA are especially suitable for soybean grown in Australia or South America, in particular in Brazil. More preferably, mixtures of B. japonicum SEMIA 5079 and SEMIA 5080 are used. B. japonicum WB74 is especially suitable for soybean grown in South America and Africa, in particular in South Africa. Strain E-109 is especially suitable for soybean grown in South America, in particular in Argentina.

The present invention also relates to mixtures, wherein the at least one pesticide II is selected from B. japonicum and further comprises a pesticide III, wherein pesticide III is selected from jasmonic acid, its salts and derivatives thereof, preferably methyl-jasmonate or cis-jasmone.

The present invention also relates to mixtures, wherein the at least one pesticide II is selected from Bradyrhizobium elkanii and Bradyrhizobium liaoningense, more preferably from B. elkanii even more preferably B. elkanii strains SEMIA 587 and SEMIA 5019; in particular mixtures of both. These mixtures are particularly suitable in soybean in Australia or South America, in particular in Brazil.

The present invention also relates to mixtures, wherein pesticide II is selected from Brady-rhizobium sp. (Arachis) and B. sp. (Vigna) which shall describe the cowpea miscellany cross-inoculation group which includes inter alia indigenous cowpea bradyrhizobia on cowpea (Vigna unguiculata), siratro (Macroptilium atropurpureum), lima bean (Phaseolus lunatus), and peanut (Arachis hypogaea), in particular in particular B. sp. (Vigna) strain PNL1. This mixture comprising as pesticide II B. sp. (Arachis) or B. sp. (Vigna) is especially suitable for use in peanut, cowpea, Mung bean, Moth bean, Dune bean, Rice bean, Snake bean and Creeping vigna, in particular peanut.

The present invention also relates to mixtures, wherein the at least one pesticide II is selected from Bradyrhizobium lupini (also called B. sp. (Lupine), B. lupines or Rhizobium lupini). These mixtures are especially suitable for use in dry beans and lupins. Preferably, B. lupini is strain LL13. This strain is especially suitable for lupins grown in Australia, North America or Europe, in particular in Europe.

The present invention also relates to mixtures wherein the at least one pesticide II is selected from Rhizobium leguminosarum bv. phaseoli especially for the legume common bean (Phaseolus vulgaris), but also for other for various legumes such as alfalfa, clover, peas, beans, lentils, soybeans, peanuts and other crops such as corn and lettuce, even more preferably strain RG-B10 thereof; R. l. bv. trifolii, especially strain RP113-7 thereof, R. l. bv. viciae, in particular strains RG-P2, SU303, WSM1455 and P1 NP3Cst thereof, in particular P1 NP3Cst; R. tropici, especially strains CC511, CIAT 899 and PRF 81 thereof; and Sinorhizobium meliloti, especially strain RCR2011 thereof. Further R. l. bv. phaseoli or R. etli strains are e. g. known from the above mentioned references and Appl. Environ. Microbiol. 45(3), 737-742, 1983; ibida 54(5), 1280-1283, 1988.

According to a further embodiment, in the inventive mixtures pesticide II is selected from one compound II selected from Sinorhizobium meliloti more preferably from RCR2011, S. meliloti NRG185, S. meliloti RRI128, S. meliloti SU277,

R. tropici s useful for a range of legume crops especially all kind of clovers e. g. in tropical regions such as Brazil. Preferably, mixtures comprise as R. tropic/at least one strain selected from CC511, CIAT899, H12 and PRF 81.

The present invention also relates to mixtures wherein the at least one pesticide II is selected from R. leguminosarum bv. phaseoli, R. l. bv. trifolii, R. l. bv. viciae, R. tropici and Sinorhizobium meliloti, and further comprises a pesticide III, wherein pesticide III is selected from jasmonic acid, its salts and derivatives thereof, preferably methyl-jasmonate or cis-jasmone.

According to a further embodiment, the at least one pesticide II is selected from Delfiia acidovorans, in particular strain RAY209, especially in soybean and canola.

Accordingly, the present invention furthermore relates to compositions comprising one compound I (component 1) and one pesticide II (component 2), which pesticide II is selected from the column “Co. 2” of the lines C-1 to C-870 of Table C.

A further embodiment relates to the compositions C-1 to C-870 listed in Table C, where a row of Table C corresponds in each case to a fungicidal composition comprising as active components one of the in the present specification individualized compounds of formula I (component 1) and the respective pesticide II from groups A) to O) (component 2) stated in the row in question. Preferably, the compositions described comprise the active components in synergistically effective amounts.

TABLE C
Compositions comprising as active components one indiviualized
compound I (I) (in Column Co. 1) and as component 2) (in Column Co. 2)
one pesticide from groups A) to O) [which is coded e.g. as (A.1.1)
for azoxystrobin as defined above].

| Mixt. | Co. 1 | Co. 2 |
|---|---|---|
| C-1 | (I) | (A.1.1) |
| C-2 | (I) | (A.1.2) |
| C-3 | (I) | (A.1.3) |
| C-4 | (I) | (A.1.4) |
| C-5 | (I) | (A.1.5) |
| C-6 | (I) | (A.1.6) |
| C-7 | (I) | (A.1.7) |
| C-8 | (I) | (A.1.8) |
| C-9 | (I) | (A.1.9) |
| C-10 | (I) | (A.1.10) |
| C-11 | (I) | (A.1.11) |
| C-12 | (I) | (A.1.12) |
| C-13 | (I) | (A.1.13) |
| C-14 | (I) | (A.1.14) |
| C-15 | (I) | (A.1.15) |
| C-16 | (I) | (A.1.16) |
| C-17 | (I) | (A.1.17) |
| C-18 | (I) | (A.1.18) |
| C-19 | (I) | (A.1.19) |
| C-20 | (I) | (A.1.20) |
| C-21 | (I) | (A.1.21) |
| C-22 | (I) | (A.1.22) |
| C-23 | (I) | (A.1.23) |
| C-24 | (I) | (A.1.24) |
| C-25 | (I) | (A.1.25) |
| C-26 | (I) | (A.1.26) |
| C-27 | (I) | (A.2.1) |
| C-28 | (I) | (A.2.2) |
| C-29 | (I) | (A.2.3) |
| C-30 | (I) | (A.2.4) |
| C-31 | (I) | (A.2.5) |
| C-32 | (I) | (A.2.6) |
| C-33 | (I) | (A.2.7) |
| C-34 | (I) | (A.3.1) |
| C-35 | (I) | (A.3.2) |
| C-36 | (I) | (A.3.3) |
| C-37 | (I) | (A.3.4) |
| C-38 | (I) | (A.3.5) |
| C-39 | (I) | (A.3.6) |
| C-40 | (I) | (A.3.7) |
| C-41 | (I) | (A.3.8) |
| C-42 | (I) | (A.3.9) |
| C-43 | (I) | (A.3.10) |
| C-44 | (I) | (A.3.11) |
| C-45 | (I) | (A.3.12) |
| C-46 | (I) | (A.3.13) |
| C-47 | (I) | (A.3.14) |
| C-48 | (I) | (A.3.15) |
| C-49 | (I) | (A.3.16) |
| C-50 | (I) | (A.3.17) |
| C-51 | (I) | (A.3.18) |
| C-52 | (I) | (A.3.19) |
| C-53 | (I) | (A.3.20) |
| C-54 | (I) | (A.3.21) |
| C-55 | (I) | (A.3.22) |
| C-56 | (I) | (A.3.23) |
| C-57 | (I) | (A.3.24) |
| C-58 | (I) | (A.3.25) |
| C-59 | (I) | (A.3.26) |
| C-60 | (I) | (A.3.27) |
| C-61 | (I) | (A.4.1) |
| C-62 | (I) | (A.4.2) |
| C-63 | (I) | (A.4.3) |
| C-64 | (I) | (A.4.4) |
| C-65 | (I) | (A.4.5) |
| C-66 | (I) | (A.4.6) |
| C-67 | (I) | (A.4.7) |
| C-68 | (I) | (A.4.8) |
| C-69 | (I) | (A.4.9) |
| C-70 | (I) | (A.4.10) |
| C-71 | (I) | (A.4.11) |
| C-72 | (I) | (A.4.12) |
| C-73 | (I) | (B.1.1) |
| C-74 | (I) | (B.1.2) |
| C-75 | (I) | (B.1.3) |
| C-76 | (I) | (B.1.4) |
| C-77 | (I) | (B.1.5) |
| C-78 | (I) | (B.1.6) |
| C-79 | (I) | (B.1.7) |
| C-80 | (I) | (B.1.8) |
| C-81 | (I) | (B.1.9) |
| C-82 | (I) | (B.1.10) |
| C-83 | (I) | (B.1.11) |
| C-84 | (I) | (B.1.12) |
| C-85 | (I) | (B.1.13) |
| C-86 | (I) | (B.1.14) |
| C-87 | (I) | (B.1.15) |
| C-88 | (I) | (B.1.16) |
| C-89 | (I) | (B.1.17) |
| C-90 | (I) | (B.1.18) |
| C-91 | (I) | (B.1.19) |
| C-92 | (I) | (B.1.20) |
| C-93 | (I) | (B.1.21) |
| C-94 | (I) | (B.1.22) |
| C-95 | (I) | (B.1.23) |
| C-96 | (I) | (B.1.24) |
| C-97 | (I) | (B.1.25) |
| C-98 | (I) | (B.1.26) |
| C-99 | (I) | (B.1.27) |
| C-100 | (I) | (B.1.28) |
| C-101 | (I) | (B.1.29) |
| C-102 | (I) | (B.1.30) |
| C-103 | (I) | (B.1.31) |
| C-104 | (I) | (B.1.32) |
| C-105 | (I) | (B.1.33) |
| C-106 | (I) | (B.1.34) |
| C-107 | (I) | (B.1.35) |
| C-108 | (I) | (B.1.36) |
| C-109 | (I) | (B.1.37) |
| C-110 | (I) | (B.1.38) |
| C-111 | (I) | (B.1.39) |
| C-112 | (I) | (B.1.40) |
| C-113 | (I) | (B.1.41) |
| C-114 | (I) | (B.1.42) |
| C-115 | (I) | (B.1.43) |
| C-116 | (I) | (B.1.44) |
| C-117 | (I) | (B.1.45) |
| C-118 | (I) | (B.1.46) |
| C-119 | (I) | (B.1.47) |
| C-120 | (I) | (B.1.48) |
| C-121 | (I) | (B.1.49) |
| C-122 | (I) | (B.1.50) |
| C-123 | (I) | (B.1.51) |
| C-124 | (I) | (B.2.1) |
| C-125 | (I) | (B.2.2) |
| C-126 | (I) | (B.2.3) |
| C-127 | (I) | (B.2.4) |
| C-128 | (I) | (B.2.5) |
| C-129 | (I) | (B.2.6) |
| C-130 | (I) | (B.2.7) |
| C-131 | (I) | (B.2.8) |
| C-132 | (I) | (B.3.1) |
| C-133 | (I) | (C.1.1) |
| C-134 | (I) | (C.1.2) |
| C-135 | (I) | (C.1.3) |
| C-136 | (I) | (C.1.4) |
| C-137 | (I) | (C.1.5) |
| C-138 | (I) | (C.1.6) |
| C-139 | (I) | (C.1.7) |
| C-140 | (I) | (C.2.1) |
| C-141 | (I) | (C.2.2) |
| C-142 | (I) | (C.2.3) |
| C-143 | (I) | (C.2.4) |
| C-144 | (I) | (C.2.5) |
| C-145 | (I) | (C.2.6) |
| C-146 | (I) | (C.2.7) |
| C-147 | (I) | (D.1.1) |
| C-148 | (I) | (D.1.2) |
| C-149 | (I) | (D.1.3) |
| C-150 | (I) | (D.1.4) |
| C-151 | (I) | (D.1.5) |
| C-152 | (I) | (D.1.6) |
| C-153 | (I) | (D.2.1) |
| C-154 | (I) | (D.2.2) |
| C-155 | (I) | (D.2.3) |
| C-156 | (I) | (D.2.4) |
| C-157 | (I) | (D.2.5) |
| C-158 | (I) | (D.2.6) |
| C-159 | (I) | (D.2.7) |
| C-160 | (I) | (E.1.1) |
| C-161 | (I) | (E.1.2) |
| C-162 | (I) | (E.1.3) |
| C-163 | (I) | (E.2.1) |
| C-164 | (I) | (E.2.2) |
| C-165 | (I) | (E.2.3) |
| C-166 | (I) | (E.2.4) |
| C-167 | (I) | (E.2.5) |
| C-168 | (I) | (E.2.6) |
| C-169 | (I) | (E.2.7) |
| C-170 | (I) | (E.2.8) |
| C-171 | (I) | (F.1.1) |
| C-172 | (I) | (F.1.2) |
| C-173 | (I) | (F.1.3) |
| C-174 | (I) | (F.1.4) |
| C-175 | (I) | (F.1.5) |
| C-176 | (I) | (F.1.6) |
| C-177 | (I) | (F.2.1) |
| C-178 | (I) | (G.1.1) |
| C-179 | (I) | (G.1.2) |
| C-180 | (I) | (G.1.3) |
| C-181 | (I) | (G.1.4) |
| C-182 | (I) | (G.2.1) |
| C-183 | (I) | (G.2.2) |
| C-184 | (I) | (G.2.3) |
| C-185 | (I) | (G.2.4) |
| C-186 | (I) | (G.2.5) |
| C-187 | (I) | (G.2.6) |
| C-188 | (I) | (G.2.7) |
| C-189 | (I) | (G.3.1) |
| C-190 | (I) | (G.3.2) |
| C-191 | (I) | (G.3.3) |
| C-192 | (I) | (G.3.4) |
| C-193 | (I) | (G.3.5) |
| C-194 | (I) | (G.3.6) |
| C-195 | (I) | (G.3.7) |
| C-196 | (I) | (G.3.8) |
| C-197 | (I) | (G.4.1) |
| C-198 | (I) | (G.5.1) |
| C-199 | (I) | (G.5.2) |
| C-200 | (I) | (G.5.3) |
| C-201 | (I) | (H.1.1) |
| C-202 | (I) | (H.1.2) |
| C-203 | (I) | (H.1.3) |
| C-204 | (I) | (H.1.4) |
| C-205 | (I) | (H.1.5) |
| C-206 | (I) | (H.1.6) |
| C-207 | (I) | (H.2.1) |
| C-208 | (I) | (H.2.2) |
| C-209 | (I) | (H.2.3) |
| C-210 | (I) | (H.2.4) |
| C-211 | (I) | (H.2.5) |
| C-212 | (I) | (H.2.6) |
| C-213 | (I) | (H.2.7) |
| C-214 | (I) | (H.2.8) |
| C-215 | (I) | (H.2.9) |
| C-216 | (I) | (H.3.1) |
| C-217 | (I) | (H.3.2) |
| C-218 | (I) | (H.3.3) |
| C-219 | (I) | (H.3.4) |
| C-220 | (I) | (H.3.5) |
| C-221 | (I) | (H.3.6) |
| C-222 | (I) | (H.3.7) |
| C-223 | (I) | (H.3.8) |
| C-224 | (I) | (H.3.9) |
| C-225 | (I) | (H.3.10) |
| C-226 | (I) | (H.3.11) |
| C-227 | (I) | (H.4.1) |
| C-228 | (I) | (H.4.2) |
| C-229 | (I) | (H.4.3) |
| C-230 | (I) | (H.4.4) |
| C-231 | (I) | (H.4.5) |
| C-232 | (I) | (H.4.6) |
| C-233 | (I) | (H.4.7) |
| C-234 | (I) | (H.4.8) |
| C-235 | (I) | (H.4.9) |
| C-236 | (I) | (H.4.10) |
| C-237 | (I) | (I.1.1) |
| C-238 | (I) | (I.1.2) |
| C-239 | (I) | (I.2.1) |
| C-240 | (I) | (I.2.2) |
| C-241 | (I) | (I.2.3) |
| C-242 | (I) | (I.2.4) |
| C-243 | (I) | (I.2.5) |
| C-244 | (I) | (J.1.1) |
| C-245 | (I) | (J.1.2) |
| C-246 | (I) | (J.1.3) |
| C-247 | (I) | (J.1.4) |
| C-248 | (I) | (J.1.5) |
| C-249 | (I) | (J.1.6) |
| C-250 | (I) | (J.1.7) |
| C-251 | (I) | (J.1.8) |
| C-252 | (I) | (J.1.9) |
| C-253 | (I) | (K.1.1) |
| C-254 | (I) | (K.1.2) |
| C-255 | (I) | (K.1.3) |
| C-256 | (I) | (K.1.4) |
| C-257 | (I) | (K.1.5) |
| C-258 | (I) | (K.1.6) |
| C-259 | (I) | (K.1.7) |
| C-260 | (I) | (K.1.8) |
| C-261 | (I) | (K.1.9) |
| C-262 | (I) | (K.1.10) |
| C-263 | (I) | (K.1.11) |
| C-264 | (I) | (K.1.12) |
| C-265 | (I) | (K.1.13) |
| C-266 | (I) | (K.1.14) |
| C-267 | (I) | (K.1.15) |
| C-268 | (I) | (K.1.16) |
| C-269 | (I) | (K.1.17) |
| C-270 | (I) | (K.1.18) |
| C-271 | (I) | (K.1.19) |
| C-272 | (I) | (K.1.20) |
| C-273 | (I) | (K.1.21) |
| C-274 | (I) | (K.1.22) |
| C-275 | (I) | (K.1.23) |
| C-276 | (I) | (K.1.24) |
| C-277 | (I) | (K.1.25) |
| C-278 | (I) | (K.1.26) |
| C-279 | (I) | (K.1.27) |
| C-280 | (I) | (K.1.28) |
| C-281 | (I) | (K.1.29) |
| C-282 | (I) | (K.1.30) |
| C-283 | (I) | (K.1.31) |
| C-284 | (I) | (K.1.32) |
| C-285 | (I) | (K.1.33) |
| C-286 | (I) | (K.1.34) |
| C-287 | (I) | (K.1.35) |
| C-288 | (I) | (K.1.36) |
| C-289 | (I) | (K.1.37) |
| C-290 | (I) | (K.1.38) |
| C-291 | (I) | (K.1.39) |
| C-292 | (I) | (K.1.40) |
| C-293 | (I) | (K.1.41) |
| C-294 | (I) | (K.1.42) |
| C-295 | (I) | (K.1.43) |
| C-296 | (I) | (K.1.44) |
| C-297 | (I) | (K.1.45) |
| C-298 | (I) | (K.1.46) |
| C-299 | (I) | (K.1.47) |
| C-300 | (I) | (K.1.48) |
| C-301 | (I) | (M.1.1) |
| C-302 | (I) | (M.1.2) |
| C-303 | (I) | (M.1.3) |
| C-304 | (I) | (M.1.4) |
| C-305 | (I) | (M.1.5) |
| C-306 | (I) | (M.1.6) |
| C-307 | (I) | (M.1.7) |
| C-308 | (I) | (M.1.8) |
| C-309 | (I) | (M.1.9) |
| C-310 | (I) | (M.1.10) |
| C-311 | (I) | (M.1.11) |
| C-312 | (I) | (M.1.12) |
| C-313 | (I) | (M.1.13) |
| C-314 | (I) | (M.1.14) |
| C-315 | (I) | (M.1.15) |
| C-316 | (I) | (M.1.16) |
| C-317 | (I) | (M.1.17) |
| C-318 | (I) | (M.1.18) |
| C-319 | (I) | (M.1.19) |
| C-320 | (I) | (M.1.20) |
| C-321 | (I) | (M.1.21) |
| C-322 | (I) | (M.1.22) |
| C-323 | (I) | (M.1.23) |
| C-324 | (I) | (M.1.24) |
| C-325 | (I) | (M.1.25) |
| C-326 | (I) | (M.1.26) |
| C-327 | (I) | (M.1.27) |
| C-328 | (I) | (M.1.28) |
| C-329 | (I) | (M.1.29) |
| C-330 | (I) | (M.1.30) |
| C-331 | (I) | (M.1.31) |
| C-332 | (I) | (M.1.32) |
| C-333 | (I) | (M.1.33) |
| C-334 | (I) | (M.1.34) |
| C-335 | (I) | (M.1.35) |
| C-336 | (I) | (M.1.36) |
| C-337 | (I) | (M.1.37) |
| C-338 | (I) | (M.1.38) |
| C-339 | (I) | (M.1.39) |
| C-340 | (I) | (M.1.40) |
| C-341 | (I) | (M.1.41) |
| C-342 | (I) | (M.1.42) |
| C-343 | (I) | (M.1.43) |
| C-344 | (I) | (M.1.44) |
| C-345 | (I) | (M.1.45) |
| C-346 | (I) | (M.1.46) |
| C-347 | (I) | (M.1.47) |
| C-348 | (I) | (M.1.48) |
| C-349 | (I) | (M.1.49) |
| C-350 | (I) | (M.1.50) |
| C-351 | (I) | (N.1.1) |
| C-352 | (I) | (N.1.2) |
| C-353 | (I) | (N.1.3) |
| C-354 | (I) | (N.1.4) |
| C-355 | (I) | (N.1.5) |
| C-356 | (I) | (N.2.1) |
| C-357 | (I) | (N.2.2) |
| C-358 | (I) | (N.2.3) |
| C-359 | (I) | (N.3.1) |
| C-360 | (I) | (N.3.2) |
| C-361 | (I) | (N.3.3) |
| C-362 | (I) | (N.3.4) |
| C-363 | (I) | (N.4.1) |
| C-364 | (I) | (N.5.1) |
| C-365 | (I) | (N.6.1) |
| C-366 | (I) | (N.6.2) |
| C-367 | (I) | (N.6.3) |
| C-368 | (I) | (N.6.4) |
| C-369 | (I) | (N.6.5) |
| C-370 | (I) | (N.7.1) |
| C-371 | (I) | (N.7.2) |
| C-372 | (I) | (N.7.3) |
| C-373 | (I) | (N.8.1) |
| C-374 | (I) | (N.9.1) |
| C-375 | (I) | (N.10.1) |
| C-376 | (I) | (N.10.2) |
| C-377 | (I) | (N.10.3) |
| C-378 | (I) | (N.10.4) |
| C-379 | (I) | (N.10.5) |
| C-380 | (I) | (N.11.1) |
| C-381 | (I) | (N.12.1) |
| C-382 | (I) | (N.12.2) |
| C-383 | (I) | (N.12.3) |
| C-384 | (I) | (N.12.4) |
| C-385 | (I) | (N.13.1) |
| C-386 | (I) | (N.13.2) |
| C-387 | (I) | (N.13.3) |
| C-388 | (I) | (N.13.4) |
| C-389 | (I) | (N.13.5) |
| C-390 | (I) | (N.13.6) |
| C-391 | (I) | (N.13.7) |
| C-392 | (I) | (N.13.8) |
| C-393 | (I) | (N.13.9) |
| C-394 | (I) | (N.14.1) |
| C-395 | (I) | (N.14.2) |
| C-396 | (I) | (N.14.3) |
| C-397 | (I) | (N.15.1) |
| C-398 | (I) | (N.16.1) |
| C-399 | (I) | (N.16.2) |
| C-400 | (I) | (N.17.1) |
| C-401 | (I) | (N.17.2) |
| C-402 | (I) | (N.17.3) |
| C-403 | (I) | (N.17.4) |
| C-404 | (I) | (N.17.5) |
| C-405 | (I) | (N.17.6) |
| C-406 | (I) | (N.17.7) |
| C-407 | (I) | (N.17.8) |
| C-408 | (I) | (N.17.9) |
| C-409 | (I) | (N.17.10) |
| C-410 | (I) | (N.17.11) |
| C-411 | (I) | (N.17.12) |
| C-412 | (I) | (O.1.1) |
| C-413 | (I) | (O.1.2) |
| C-414 | (I) | (O.1.3) |
| C-415 | (I) | (O.1.4) |
| C-416 | (I) | (O.1.5) |
| C-417 | (I) | (O.1.6) |
| C-418 | (I) | (O.1.7) |
| C-419 | (I) | (O.1.8) |
| C-420 | (I) | (O.1.9) |
| C-421 | (I) | (O.1.10) |
| C-422 | (I) | (O.1.11) |
| C-423 | (I) | (O.1.12) |
| C-424 | (I) | (O.1.13) |
| C-425 | (I) | (O.1.14) |
| C-426 | (I) | (O.1.15) |
| C-427 | (I) | (O.1.16) |
| C-428 | (I) | (O.1.17) |
| C-429 | (I) | (O.1.18) |
| C-430 | (I) | (O.1.19) |
| C-431 | (I) | (O.1.20) |
| C-432 | (I) | (O.1.21) |
| C-433 | (I) | (O.1.22) |
| C-434 | (I) | (O.1.23) |
| C-435 | (I) | (O.1.24) |
| C-436 | (I) | (O.1.25) |
| C-437 | (I) | (O.1.26) |
| C-438 | (I) | (O.1.27) |
| C-439 | (I) | (O.1.28) |
| C-440 | (I) | (O.1.29) |
| C-441 | (I) | (O.1.30) |
| C-442 | (I) | (O.1.31) |
| C-443 | (I) | (O.1.32) |
| C-444 | (I) | (O.1.33) |
| C-445 | (I) | (O.1.34) |
| C-446 | (I) | (O.1.35) |
| C-447 | (I) | (O.1.36) |
| C-448 | (I) | (O.1.37) |
| C-449 | (I) | (O.1.38) |
| C-450 | (I) | (O.2.1) |
| C-451 | (I) | (O.2.2) |
| C-452 | (I) | (O.2.3) |
| C-453 | (I) | (O.2.4) |
| C-454 | (I) | (O.2.5) |
| C-455 | (I) | (O.2.6) |
| C-456 | (I) | (O.2.7) |
| C-457 | (I) | (O.2.8) |
| C-458 | (I) | (O.2.9) |
| C-459 | (I) | (O.2.10) |
| C-460 | (I) | (O.2.11) |
| C-461 | (I) | (O.2.12) |
| C-462 | (I) | (O.2.13) |
| C-463 | (I) | (O.2.14) |
| C-464 | (I) | (O.2.15) |
| C-465 | (I) | (O.2.16) |
| C-466 | (I) | (O.3.1) |
| C-467 | (I) | (O.3.2) |
| C-468 | (I) | (O.3.3) |
| C-469 | (I) | (O.3.4) |
| C-470 | (I) | (O.3.5) |
| C-471 | (I) | (O.3.6) |
| C-472 | (I) | (O.3.7) |
| C-473 | (I) | (O.3.8) |
| C-474 | (I) | (O.3.9) |
| C-475 | (I) | (O.3.10) |
| C-476 | (I) | (O.3.11) |
| C-477 | (I) | (O.3.12) |
| C-478 | (I) | (O.3.13) |
| C-479 | (I) | (O.3.14) |
| C-480 | (I) | (O.3.15) |
| C-481 | (I) | (O.3.16) |
| C-482 | (I) | (O.3.17) |
| C-483 | (I) | (O.3.18) |
| C-484 | (I) | (O.3.19) |
| C-485 | (I) | (O.3.20) |
| C-486 | (I) | (O.3.21) |
| C-487 | (I) | (O.3.22) |
| C-488 | (I) | (O.3.23) |
| C-489 | (I) | (O.3.24) |
| C-490 | (I) | (O.3.25) |
| C-491 | (I) | (O.3.26) |
| C-492 | (I) | (O.3.27) |
| C-493 | (I) | (O.4.1) |
| C-494 | (I) | (O.4.2) |
| C-495 | (I) | (O.4.3) |
| C-496 | (I) | (O.4.4) |
| C-497 | (I) | (O.4.5) |
| C-498 | (I) | (O.4.6) |
| C-499 | (I) | (O.4.7) |
| C-500 | (I) | (O.4.8) |
| C-501 | (I) | (O.4.9) |
| C-502 | (I) | (O.4.10) |
| C-503 | (I) | (O.4.11) |
| C-504 | (I) | (O.4.12) |
| C-505 | (I) | (O.4.13) |
| C-506 | (I) | (O.4.14) |
| C-507 | (I) | (O.4.15) |
| C-508 | (I) | (O.4.16) |
| C-509 | (I) | (O.4.17) |
| C-510 | (I) | (O.4.18) |
| C-511 | (I) | (O.4.19) |
| C-512 | (I) | (O.4.20) |
| C-513 | (I) | (O.4.21) |
| C-514 | (I) | (O.4.22) |
| C-515 | (I) | (O.4.23) |
| C-516 | (I) | (O.4.24) |
| C-517 | (I) | (O.5.1) |
| C-518 | (I) | (O.5.2) |
| C-519 | (I) | (O.5.3) |
| C-520 | (I) | (O.5.4) |
| C-521 | (I) | (O.5.5) |
| C-522 | (I) | (O.5.6) |
| C-523 | (I) | (O.5.7) |
| C-524 | (I) | (O.5.8) |
| C-525 | (I) | (O.5.9) |
| C-526 | (I) | (O.6.1) |
| C-527 | (I) | (O.6.2) |
| C-528 | (I) | (O.6.3) |
| C-529 | (I) | (O.6.4) |
| C-530 | (I) | (O.6.5) |
| C-531 | (I) | (O.6.6) |
| C-532 | (I) | (O.6.7) |
| C-533 | (I) | (O.7.1) |
| C-534 | (I) | (O.7.2) |
| C-535 | (I) | (O.7.3) |
| C-536 | (I) | (O.7.4) |
| C-537 | (I) | (O.7.5) |
| C-538 | (I) | (O.7.6) |
| C-539 | (I) | (O.8.1) |
| C-540 | (I) | (O.8.2) |
| C-541 | (I) | (O.8.3) |
| C-542 | (I) | (O.8.4) |
| C-543 | (I) | (O.8.5) |
| C-544 | (I) | (O.9.1) |
| C-545 | (I) | (O.9.2) |
| C-546 | (I) | (O.9.3) |
| C-547 | (I) | (O.10.1) |
| C-548 | (I) | (O.11.1) |
| C-549 | (I) | (O.11.2) |
| C-550 | (I) | (O.11.3) |
| C-551 | (I) | (O.11.4) |
| C-552 | (I) | (O.12.1) |
| C-553 | (I) | (O.13.1) |
| C-554 | (I) | (O.14.1) |
| C-555 | (I) | (O.14.2) |
| C-556 | (I) | (O.15.1) |
| C-557 | (I) | (O.15.2) |
| C-558 | (I) | (O.15.3) |
| C-559 | (I) | (O.15.4) |
| C-560 | (I) | (O.15.5) |
| C-561 | (I) | (O.15.6) |
| C-562 | (I) | (O.15.7) |
| C-563 | (I) | (O.15.8) |
| C-564 | (I) | (O.15.9) |
| C-565 | (I) | (O.15.10) |
| C-566 | (I) | (O.15.11) |
| C-567 | (I) | (O.16.1) |
| C-568 | (I) | (O.16.2) |
| C-569 | (I) | (O.16.3) |
| C-570 | (I) | (O.16.4) |
| C-571 | (I) | (O.16.5) |
| C-572 | (I) | (O.16.6) |
| C-573 | (I) | (L.1.1) |
| C-574 | (I) | (L.1.2) |
| C-575 | (I) | (L.1.3) |
| C-576 | (I) | (L.1.4) |
| C-577 | (I) | (L.1.5) |
| C-578 | (I) | (L.1.6) |
| C-579 | (I) | (L.1.7) |
| C-580 | (I) | (L.1.8) |
| C-581 | (I) | (L.1.9) |
| C-582 | (I) | (L.1.10) |
| C-583 | (I) | (L.1.11) |
| C-584 | (I) | (L.1.12) |
| C-585 | (I) | (L.1.13) |
| C-586 | (I) | (L.1.14) |
| C-587 | (I) | (L.1.15) |
| C-588 | (I) | (L.1.16) |
| C-589 | (I) | (L.1.17) |
| C-590 | (I) | (L.1.18) |
| C-591 | (I) | (L.1.19) |
| C-592 | (I) | (L.1.20) |
| C-593 | (I) | (L.1.21) |
| C-594 | (I) | (L.1.22) |
| C-595 | (I) | (L.1.23) |
| C-596 | (I) | (L.1.24) |
| C-597 | (I) | (L.1.25) |
| C-598 | (I) | (L.1.26) |
| C-599 | (I) | (L.1.27) |
| C-600 | (I) | (L.1.28) |
| C-601 | (I) | (L.1.29) |
| C-602 | (I) | (L.1.30) |
| C-603 | (I) | (L.1.31) |
| C-604 | (I) | (L.1.32) |
| C-605 | (I) | (L.1.33) |
| C-606 | (I) | (L.1.34) |
| C-607 | (I) | (L.1.35) |
| C-608 | (I) | (L.1.36) |
| C-609 | (I) | (L.1.37) |
| C-610 | (I) | (L.1.38) |
| C-611 | (I) | (L.1.39) |
| C-612 | (I) | (L.1.40) |
| C-613 | (I) | (L.1.41) |
| C-614 | (I) | (L.1.42) |
| C-615 | (I) | (L.1.43) |
| C-616 | (I) | (L.1.44) |
| C-617 | (I) | (L.1.45) |
| C-618 | (I) | (L.1.46) |
| C-619 | (I) | (L.1.47) |
| C-620 | (I) | (L.1.48) |
| C-621 | (I) | (L.1.49) |
| C-622 | (I) | (L.1.50) |
| C-623 | (I) | (L.1.51) |
| C-624 | (I) | (L.1.52) |
| C-625 | (I) | (L.1.53) |
| C-626 | (I) | (L.1.54) |
| C-627 | (I) | (L.1.55) |
| C-628 | (I) | (L.1.56) |
| C-629 | (I) | (L.1.57) |
| C-630 | (I) | (L.1.58) |
| C-631 | (I) | (L.1.59) |
| C-632 | (I) | (L.1.60) |
| C-633 | (I) | (L.1.61) |
| C-634 | (I) | (L.1.62) |
| C-635 | (I) | (L.1.63) |
| C-636 | (I) | (L.1.64) |
| C-637 | (I) | (L.1.65) |
| C-638 | (I) | (L.1.66) |
| C-639 | (I) | (L.1.67) |
| C-640 | (I) | (L.1.68) |
| C-641 | (I) | (L.1.69) |
| C-642 | (I) | (L.1.70) |
| C-643 | (I) | (L.1.71) |
| C-644 | (I) | (L.1.72) |
| C-645 | (I) | (L.1.73) |
| C-646 | (I) | (L.1.74) |
| C-647 | (I) | (L.1.75) |
| C-648 | (I) | (L.1.76) |
| C-649 | (I) | (L.1.77) |
| C-650 | (I) | (L.1.78) |
| C-651 | (I) | (L.1.79) |
| C-652 | (I) | (L.1.80) |
| C-653 | (I) | (L.1.81) |
| C-654 | (I) | (L.1.82) |
| C-655 | (I) | (L.1.83) |
| C-656 | (I) | (L.1.84) |
| C-657 | (I) | (L.1.85) |
| C-658 | (I) | (L.1.86) |
| C-659 | (I) | (L.1.87) |
| C-660 | (I) | (L.1.88) |
| C-661 | (I) | (L.1.89) |
| C-662 | (I) | (L.1.90) |
| C-663 | (I) | (L.1.91) |
| C-664 | (I) | (L.1.92) |
| C-665 | (I) | (L.1.93) |
| C-666 | (I) | (L.1.94) |
| C-667 | (I) | (L.1.95) |
| C-668 | (I) | (L.1.96) |
| C-669 | (I) | (L.1.97) |
| C-670 | (I) | (L.2.1) |
| C-671 | (I) | (L.2.2) |
| C-672 | (I) | (L.2.3) |
| C-673 | (I) | (L.2.4) |
| C-674 | (I) | (L.2.5) |
| C-675 | (I) | (L.2.6) |
| C-676 | (I) | (L.2.7) |
| C-677 | (I) | (L.2.8) |
| C-678 | (I) | (L.2.9) |
| C-679 | (I) | (L.2.10) |
| C-680 | (I) | (L.2.11) |
| C-681 | (I) | (L.3.1) |
| C-682 | (I) | (L.3.2) |
| C-683 | (I) | (L.3.3) |
| C-684 | (I) | (L.3.4) |
| C-685 | (I) | (L.3.5) |
| C-686 | (I) | (L.3.6) |
| C-687 | (I) | (L.3.7) |
| C-688 | (I) | (L.3.8) |
| C-689 | (I) | (L.3.9) |
| C-690 | (I) | (L.3.10) |
| C-691 | (I) | (L.3.11) |
| C-692 | (I) | (L.3.12) |
| C-693 | (I) | (L.3.13) |
| C-694 | (I) | (L.3.14) |
| C-695 | (I) | (L.3.15) |
| C-696 | (I) | (L.3.16) |
| C-697 | (I) | (L.3.17) |
| C-698 | (I) | (L.3.18) |
| C-699 | (I) | (L.3.19) |
| C-700 | (I) | (L.3.20) |
| C-701 | (I) | (L.3.21) |
| C-702 | (I) | (L.3.22) |
| C-703 | (I) | (L.3.23) |
| C-704 | (I) | (L.3.24) |
| C-705 | (I) | (L.3.25) |
| C-706 | (I) | (L.3.26) |
| C-707 | (I) | (L.3.27) |
| C-708 | (I) | (L.3.28) |
| C-709 | (I) | (L.3.29) |
| C-710 | (I) | (L.3.30) |
| C-711 | (I) | (L.3.31) |
| C-712 | (I) | (L.3.32) |
| C-713 | (I) | (L.3.33) |
| C-714 | (I) | (L.3.34) |
| C-715 | (I) | (L.3.35) |
| C-716 | (I) | (L.3.36) |
| C-717 | (I) | (L.3.37) |
| C-718 | (I) | (L.3.38) |
| C-719 | (I) | (L.3.39) |
| C-720 | (I) | (L.3.40) |
| C-721 | (I) | (L.3.41) |
| C-722 | (I) | (L.3.42) |
| C-723 | (I) | (L.3.43) |
| C-724 | (I) | (L.3.44) |
| C-725 | (I) | (L.3.45) |
| C-726 | (I) | (L.3.46) |
| C-727 | (I) | (L.3.47) |
| C-728 | (I) | (L.3.48) |
| C-729 | (I) | (L.3.49) |
| C-730 | (I) | (L.3.50) |
| C-731 | (I) | (L.3.51) |
| C-732 | (I) | (L.3.52) |
| C-733 | (I) | (L.3.53) |
| C-734 | (I) | (L.3.54) |
| C-735 | (I) | (L.3.55) |
| C-736 | (I) | (L.3.56) |
| C-737 | (I) | (L.3.57) |
| C-738 | (I) | (L.3.58) |
| C-739 | (I) | (L.3.59) |
| C-740 | (I) | (L.3.60) |
| C-741 | (I) | (L.4.1) |
| C-742 | (I) | (L.4.2) |
| C-743 | (I) | (L.4.3) |
| C-744 | (I) | (L.4.4) |
| C-745 | (I) | (L.4.5) |
| C-746 | (I) | (L.4.6) |
| C-747 | (I) | (L.4.7) |
| C-748 | (I) | (L.4.8) |
| C-749 | (I) | (L.4.9) |
| C-750 | (I) | (L.4.10) |
| C-751 | (I) | (L.4.11) |
| C-752 | (I) | (L.4.12) |
| C-753 | (I) | (L.4.13) |
| C-754 | (I) | (L.4.14) |
| C-755 | (I) | (L.4.15) |
| C-756 | (I) | (L.4.16) |
| C-757 | (I) | (L.4.17) |
| C-758 | (I) | (L.4.18) |
| C-759 | (I) | (L.4.19) |
| C-760 | (I) | (L.4.20) |
| C-761 | (I) | (L.4.21) |
| C-762 | (I) | (L.4.22) |
| C-763 | (I) | (L.4.23) |
| C-764 | (I) | (L.4.24) |
| C-765 | (I) | (L.4.25) |
| C-766 | (I) | (L.4.26) |
| C-767 | (I) | (L.4.27) |
| C-768 | (I) | (L.4.28) |
| C-769 | (I) | (L.4.29) |
| C-770 | (I) | (L.4.30) |
| C-771 | (I) | (L.4.31) |
| C-772 | (I) | (L.4.32) |
| C-773 | (I) | (L.4.33) |
| C-774 | (I) | (L.5.1) |
| C-775 | (I) | (L.5.2) |
| C-776 | (I) | (L.5.3) |
| C-777 | (I) | (L.5.4) |
| C-778 | (I) | (L.5.5) |
| C-779 | (I) | (L.5.6) |
| C-780 | (I) | (L.5.7) |
| C-781 | (I) | (L.5.8) |
| C-782 | (I) | (L.5.9) |
| C-783 | (I) | (L.5.10) |
| C-784 | (I) | (L.5.11) |
| C-785 | (I) | (L.5.12) |
| C-786 | (I) | (L.5.13) |
| C-787 | (I) | (L.5.14) |
| C-788 | (I) | (L.5.15) |
| C-789 | (I) | (L.5.16) |
| C-790 | (I) | (L.5.17) |
| C-791 | (I) | (L.5.18) |
| C-792 | (I) | (L.5.19) |
| C-793 | (I) | (L.5.20) |
| C-794 | (I) | (L.5.21) |
| C-795 | (I) | (L.5.22) |
| C-796 | (I) | (L.5.23) |
| C-797 | (I) | (L.5.24) |
| C-798 | (I) | (L.5.25) |
| C-799 | (I) | (L.5.26) |
| C-800 | (I) | (L.5.27) |
| C-801 | (I) | (L.5.28) |
| C-802 | (I) | (L.5.29) |
| C-803 | (I) | (L.5.30) |
| C-804 | (I) | (L.5.31) |
| C-805 | (I) | (L.5.32) |
| C-806 | (I) | (L.5.33) |
| C-807 | (I) | (L.5.34) |
| C-808 | (I) | (L.5.35) |
| C-809 | (I) | (L.5.36) |
| C-810 | (I) | (L.5.37) |
| C-811 | (I) | (L.5.38) |
| C-812 | (I) | (L.5.39) |
| C-813 | (I) | (L.5.40) |
| C-814 | (I) | (L.5.41) |
| C-815 | (I) | (L.5.42) |
| C-816 | (I) | (L.5.43) |
| C-817 | (I) | (L.5.44) |
| C-818 | (I) | (L.5.45) |
| C-819 | (I) | (L.5.46) |
| C-820 | (I) | (L.5.47) |
| C-821 | (I) | (L.5.48) |
| C-822 | (I) | (L.5.49) |
| C-823 | (I) | (L.5.50) |
| C-824 | (I) | (L.5.51) |
| C-825 | (I) | (L.5.52) |
| C-826 | (I) | (L.5.53) |
| C-827 | (I) | (L.5.54) |
| C-828 | (I) | (L.5.55) |
| C-829 | (I) | (L.5.56) |
| C-830 | (I) | (L.5.57) |
| C-831 | (I) | (L.5.58) |
| C-832 | (I) | (L.5.59) |
| C-833 | (I) | (L.5.60) |
| C-834 | (I) | (L.5.60) |
| C-835 | (I) | (L.5.60) |
| C-836 | (I) | (L.5.60) |
| C-837 | (I) | (L.5.60) |
| C-838 | (I) | (L.5.61) |
| C-839 | (I) | (L.5.62) |
| C-840 | (I) | (L.5.63) |
| C-841 | (I) | (L.5.64) |
| C-842 | (I) | (L.5.65) |
| C-843 | (I) | (L.5.66) |
| C-844 | (I) | (L.5.67) |
| C-845 | (I) | (L.5.67) |
| C-846 | (I) | (L.5.67) |
| C-847 | (I) | (L.5.68) |
| C-848 | (I) | (L.5.69) |
| C-849 | (I) | (L.5.70) |
| C-850 | (I) | (L.5.71) |
| C-851 | (I) | (L.5.72) |
| C-852 | (I) | (L.5.73) |
| C-853 | (I) | (L.5.74) |
| C-854 | (I) | (L.5.75) |
| C-855 | (I) | (L.6.1) |
| C-856 | (I) | (L.6.2) |
| C-857 | (I) | (L.6.3) |
| C-858 | (I) | (L.6.4) |
| C-859 | (I) | (L.6.5) |
| C-860 | (I) | (L.6.6) |
| C-861 | (I) | (L.6.7) |
| C-862 | (I) | (L.6.8) |
| C-863 | (I) | (L.6.9) |
| C-864 | (I) | (L.6.10) |
| C-865 | (I) | (L.6.11) |
| C-866 | (I) | (L.6.12) |
| C-867 | (I) | (L.6.13) |
| C-868 | (I) | (L.6.14) |
| C-869 | (I) | (L.6.15) |
| C-870 | (I) | (L.6.16) |

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, WO 13/127704, WO 13/024009, WO 13/024010 and WO 13/047441, WO 13/162072, WO 13/092224).

The mixtures of active substances can be prepared as compositions comprising besides the active ingredients at least one inert ingredient (auxiliary) by usual means, e. g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, respectively.

According to one embodiment, the microbial pesticides selected from groups L1), L3) and L5) embrace not only the isolated, pure cultures of the respective micro-organism as defined herein, but also its cell-free extract, its suspensions in a whole broth culture or as a metabolite-containing culture medium or a purified metabolite obtained from a whole broth culture of the microorganism or microorganism strain.

According to a further embodiment, the microbial pesticides selected from groups L1), L3 and L5) embraces not only the isolated, pure cultures of the respective micro-organism as defined herein, but also a cell-free extract thereof or at least one metabolite thereof, and/or a mutant of the respective micro-organism having all the identifying characteristics thereof and also a cell-free extract or at least one metabolite of the mutant.

As used herein, “whole culture broth” refers to a liquid culture of a microorganism containing vegetative cells and/or spores suspended in the culture medium and optionally metabolites produced by the respective microorganism.

As used herein, “culture medium”, refers to a medium obtainable by culturing the microorganism in said medium, preferably a liquid broth, and remaining when cells grown in the medium are removed, e. g., the supernatant remaining when cells grown in a liquid broth are removed by centrifugation, filtration, sedimentation, or other means well known in the art; comprising e. g. metabolites produced by the respective microorganism and secreted into the culture medium. The “culture medium” sometimes also referred to as “supernatant” can be obtained e. g. by centrifugation at temperatures of about 2 to 30° C. (more preferably at temperatures of 4 to 20° C.) for about 10 to 60 min (more preferably about 15 to 30 min) at about 5,000 to 20,000×g (more preferably at about 15,000×g).

As used herein, “cell-free extract” refers to an extract of the vegetative cells, spores and/or the whole culture broth of a microorganism comprising cellular metabolites produced by the respective microorganism obtainable by cell disruption methods known in the art such as solvent-based (e. g. organic solvents such as alcohols sometimes in combination with suitable salts), temperature-based, application of shear forces, cell disruption with an ultrasonicator. The desired extract may be concentrated by conventional concentration techniques such as drying, evaporation, centrifugation or alike. Certain washing steps using organic solvents and/or water-based media may also be applied to the crude extract preferably prior to use.

As used herein, the term “metabolite” refers to any component, compound, substance or byproduct (including but not limited to small molecule secondary metabolites, polyketides, fatty acid synthase products, non-ribosomal peptides, ribosomal peptides, proteins and enzymes) produced by a microorganism (such as fungi and bacteria, in particular the strains of the invention) that has any beneficial effect as described herein such as pesticidal activity or improvement of plant growth, water use efficiency of the plant, plant health, plant appearance, or the population of beneficial microorganisms in the soil around the plant activity herein.

As used herein, “isolate” refers to a pure microbial culture separated from its natural origin, such an isolate obtained by culturing a single microbial colony. An isolate is a pure culture derived from a heterogeneous, wild population of microorganisms.

As used herein, “strain” refers to isolate or a group of isolates exhibiting phenotypic and/or genotypic traits belonging to the same lineage, distinct from those of other isolates or strains of the same species.

The term “mutant” refers a microorganism obtained by direct mutant selection but also includes microorganisms that have been further mutagenized or otherwise manipulated (e. g., via the introduction of a plasmid). Accordingly, embodiments include mutants, variants, and or derivatives of the respective microorganism, both naturally occurring and artificially induced mutants. For example, mutants may be induced by subjecting the microorganism to known mutagens, such as N-methyl-nitrosoguanidine, using conventional methods.

In the case of mixtures comprising microbial pesticides II selected from groups L1), L3) and L5), the microorganisms as used according to the invention can be cultivated continuously or discontinuously in the batch process or in the fed batch or repeated fed batch process. A review of known methods of cultivation will be found in the textbook by Chmiel (Bioprozesstechnik 1. Einführung in die Bioverfahrenstechnik (Gustav Fischer Verlag, Stuttgart, 1991)) or in the textbook by Storhas (Bioreaktoren and periphere Einrichtungen (Vieweg Verlag, Braunschweig/Wiesbaden, 1994)).

When living microorganisms, such as pesticides II from groups L1), L3) and L5), form part of the compositions, such compositions can be prepared as compositions comprising besides the active ingredients at least one auxiliary (inert ingredient) by usual means (see e. g. H. D. Burges: Formulation of Micobial Biopestcides, Springer, 1998). Suitable customary types of such compositions are suspensions, dusts, powders, pastes, granules, pressings, capsules, and mixtures thereof. Examples for composition types are suspensions (e. g. SC, OD, FS), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). Herein, it has to be taken into account that each formulation type or choice of auxiliary should not influence the viability of the microorganism during storage of the composition and when finally applied to the soil, plant or plant propagation material. Suitable formulations are e. g. mentioned in WO 2008/002371, U.S. Pat. No. 6,955,912, U.S. Pat. No. 5,422,107.

Examples for suitable auxiliaries are those mentioned earlier herein, wherein it must be taken care that choice and amounts of such auxiliaries should not influence the viability of the microbial pesticides in the composition. Especially for bactericides and solvents, compatibility with the respective microorganism of the respective microbial pesticide has to be taken into account. In addition, compositions with microbial pesticides may further contain stabilizers or nutrients and UV protectants. Suitable stabilizers or nutrients are e. g. alpha-tocopherol, trehalose, glutamate, potassium sorbate, various sugars like glucose, sucrose, lactose and maltodextrine (H. D. Burges: Formulation of Micobial Biopestcides, Springer, 1998). Suitable UV protectants are e. g. inorganic compounds like titan dioxide, zinc oxide and iron oxide pigments or organic compounds like benzophenones, benzotriazoles and phenyltriazines. The compositions may in addition to auxiliaries mentioned for compositions comprising compounds I herein optionally comprise 0.1 80% stabilizers or nutrients and 0.1-10% UV protectants.

### I. SYNTHESIS EXAMPLES

With appropriate modification of the starting materials, the procedures given in the synthesis description were used to obtain further compounds. The compounds obtained in this manner are listed in the table that follows, together with physical data.

The products shown below were characterized by melting point determination, by NMR spectroscopy or by the masses ([m/z]) or retention time (RT; [min.]) determined by HPLC MS or HPLC spectrometry.

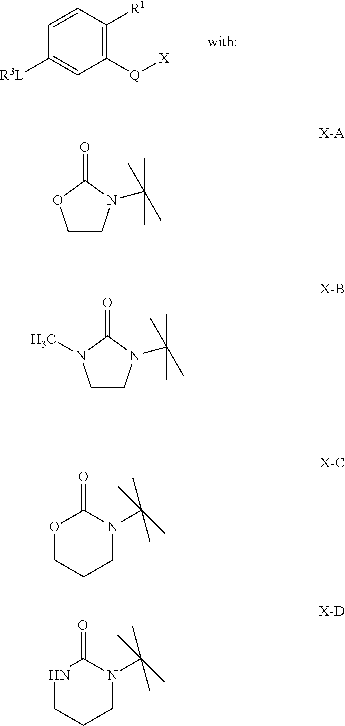

| | | | | | physical data (HPLC/ | | |
|---|---|---|---|---|---|---|---|
| | | | | | LC-MS) | | |
| | | | | | | Rt | |
| No. | R¹ | X | R³L | Q | method | [min] | m/z |
| F-1 | Cl | X-A | C-methyl-N-[(6-methyl-2- | CH₂ | B | 0.792 | 374.1 |
| | | | pyridyl)methoxy]carbonimidoyl | | | | |
| F-2 | Cl | R₂-B | C-methyl-N-[(6-methyl-2- | CH₂ | B | 0.818 | 387.6 |
| | | | pyridyl)methoxy]carbonimidoyl | | | | |
| F-3 | Cl | R₂-D | C-methyl-N-[(6-methyl-2- | CH₂ | B | 0.790 | 387.6 |
| | | | pyridyl)methoxy]carbonimidoyl | | | | |
| F-4 | Cl | R₂-C | C-methyl-N-[(6-methyl-2- | CH₂ | B | 0.784 | 388.6 |
| | | | pyridyl)methoxy]carbonimidoyl | | | | |
| F-5 | Cl | R₂-A | [1-(4-chlorophenyl)pyrazol-3-yl]oxymethyl | CH₂ | B | 1.262 | 417.8 |
| F-6 | Cl | R₂-A | [1-(4-chlorophenyl)pyrazol-3-yl]methyl | CH₂ | A | 3.55 | 402.1 |
| F-7 | CN | R₂-A | 3,4-dimethylphenyl | CH₂ | A | 3.23 | 307.1 |
| F-8 | Cl | R₂-C | 2-(2,4,6-trifluorophenyl)ethyl | NH | A | 3.52 | 385.1 |
| F-9 | Cl | R₂-B | [1-(3,5-dichlorophenyl)ethylideneamino]oxymethyl | CH₂ | A | 3.56 | 441.9 |
| F-10 | Cl | R₂-A | [[amino-[4- | CH₂ | A | 3.65 | 374.1 |
| | | | (methyl)phenyl]methylene]amino]oxymethyl | | | | |
| F-11 | Cl | R₂-A | [1-(3-chloro-4-fluoro-phenyl)pyrazol-3-yl]oxymethyl | NCH₃ | B | 1.288 | 453.4 |
| F-12 | Cl | R₂-A | [1-(2,2-difluoro-1,3-benzodioxol-5-yl)pyrazol-3- | NCH₃ | B | 1.301 | 479.5 |
| | | | yl]oxymethyl | | | | |
| F-13 | Cl | R₂-A | [1-(2,4-dichlorophenyl)-1,2,4-triazol-3-yl]oxymethyl | NCH₃ | B | 1.177 | 469.6 |
| F-14 | Cl | R₂-A | [1-(4-ethylphenyl)pyrazol-3-yl]oxymethyl | NCH₃ | B | 1.309 | 427.7 |
| F-15 | Cl | R₂-A | [1-(5-chloro-2-pyridyl)-1,2,4-triazol-3-yl]oxymethyl | NCH₃ | B | 1.133 | 435.6 |
| F-16 | Cl | R₂-A | [1-[2-chloro-4-(trifluoromethyl)phenyl]-1,2,4-triazol- | NCH₃ | B | 1.217 | 502.7 |
| | | | 3-yl]oxymethyl | | | | |
| F-17 | Cl | R₂-A | 2-pyridyloxymethyl | NCH₃ | B | 0.781 | 333.7 |
| F-18 | Cl | R₂-A | [1-[4-(trifluoromethyl)phenyl]pyrazol-3-yl]oxymethyl | NCH₃ | B | 1.309 | 467.7 |
| F-19 | Cl | R₂-A | [2-chloro-4-(C-ethyl-N-methoxy-carbonimidoyl)-5- | NCH₃ | B | 1.358 | 466.7 |
| | | | methyl-phenoxy]methyl | | | | |
| F-20 | Cl | R₂-A | [4-(4-bromophenyl)phenoxy]methyl | NCH₃ | B | 1.410 | 489.5 |
| F-21 | Cl | R₂-A | [1-[3-(trifluoromethyl)phenyl]pyrazol-3-yl]oxymethyl | NCH₃ | B | 1.303 | 467.7 |
| F-22 | Cl | R₂-A | [1-(3-chloro-4-fluoro-phenyl)pyrazol-3-yl]oxymethyl | CH₂ | B | 1.256 | 437.5 |
| F-23 | Cl | R₂-A | [1-(4-phenoxyphenyl)pyrazol-3-yl]oxymethyl | CH₂ | B | 1.321 | 475.9 |
| F-24 | Cl | R₂-A | [1-(5-chloro-2-pyridyl)-1,2,4-triazol-3-yl]oxymethyl | CH₂ | B | 1.106 | 421.5 |
| F-25 | Cl | R₂-A | [2-methyl-5-(p-tolyl)-1,2,4-triazol-3-yl]oxymethyl | CH₂ | B | 1.152 | 412.8 |
| F-26 | Cl | R₂-A | [4-(2,4-dichlorophenyl)thiazol-2-yl]oxymethyl | CH₂ | B | 1.404 | 468.7 |
| F-27 | Cl | R₂-A | [1-(p-tolyl)pyrazol-3-yl]oxymethyl | CH₂ | B | 1.220 | 397.8 |
| F-28 | Cl | R₂-A | (6-cyclopropyl-2-pyridyl)oxymethyl | CH₂ | B | 1.170 | 358.8 |
| F-29 | Cl | R₂-A | [1-[4-(trifluoromethyl)phenyl]pyrazol-3-yl]oxymethyl | CH₂ | B | 1.273 | 451.8 |
| F-30 | Cl | R₂-A | [2-methoxy-6-(trifluoromethyl)pyrimidin-4- | CH₂ | B | 1.175 | 417.8 |
| | | | yl]oxymethyl | | | | |
| F-31 | Cl | R₂-A | [4-(N-methoxy-C-methyl- | CH₂ | B | 1.202 | 388.8 |
| | | | carbonimidoyl)phenoxy]methyl | | | | |
| F-32 | Cl | R₂-A | [4-(2,4-difluorophenyl)phenoxy]methyl | CH₂ | B | 1.306 | 429.8 |
| F-33 | F | R₂-A | [1-(2-chlorophenyl)pyrazol-3-yl]oxymethyl | CH₂ | B | 1.149 | 401.8 |
| F-34 | Cl | R₂-A | [6-(trifluoromethyl)-2-pyridyl]oxy | CH₂ | A | 3.29 | 395.0 |
| F-35 | F | R₂-B | 1-(p-tolyl)pyrazol-3-yl | CH₂ | A | 3.65 | 365.2 |
| F-36 | Cl | R₂-A | 6-(4-fluorophenoxy)pyrimidin-4-yl | CH₂ | A | 3.35 | 400.0 |
| F-37 | Cl | R₂-A | [6-(2-cyanophenoxy)pyrimidin-4-yl]oxy | CH₂ | A | 3.06 | 423.1 |
| F-38 | Cl | R₂-A | [[2-[4-(N-methoxy-C-methyl-carbonimidoyl)-2,5- | CH₂ | A | 3.77 | 472.2 |
| | | | dimethyl-phenyl]-1-methyl- | | | | |
| | | | ethylidene]amino]oxymethyl | | | | |
| F-39 | Cl | R₂-A | [[1-methyl-2-[(6-methyl-2- | CH₂ | A | 3.49 | 431.1 |
| | | | pyridyl)oxyimino]propylidene]amino]oxymethyl | | | | |
| F-40 | F | R₂-B | 4-(N-methoxy-C-methyl-carbonimidoyl)-2,5- | CH₂ | A | 3.70 | 400.2 |
| | | | dimethyl-phenoxy | | | | |
| F-41 | Cl | X-A | N-[(3,5-dimethylphenyl)methoxy]-C-methyl- | NH | B | 1.281 | 387.4 |
| | | | carbonimidoyl | | | | |
| F-42 | Cl | X-A | C-methyl-N-[[5-(trifluoromethyl)-2- | NH | B | 1.192 | 428.4 |
| | | | pyridyl]methoxy]carbonimidoyl | | | | |
| F-43 | Cl | X-A | N-[(3,4-dichlorophenyl)methoxy]-C-methyl- | CH₂ | B | 1.412 | 428.8 |
| | | | carbonimidoyl | | | | |
| F-44 | Cl | X-A | N-[[3-(4-fluorophenyl)isoxazol-5-yl]methoxy]-C- | CH₂ | B | 1.349 | 443.5 |
| | | | methyl-carbonimidoyl | | | | |
| F-45 | Cl | X-A | N-[(3,5-dimethylphenyl)methoxy]-C-methyl- | CH₂ | B | 1.399 | 386.4 |
| | | | carbonimidoyl | | | | |
| F-46 | Cl | X-A | N-[(6-chloro-2-pyridyl)methoxy]-C-methyl- | CH₂ | B | 1.206 | 395.8 |
| | | | carbonimidoyl | | | | |
| F-47 | F | X-B | N-[(4-chlorophenyl)methoxy]-C-methyl- | CH₂ | B | 1.325 | 389.8 |
| | | | carbonimidoyl | | | | |
| F-48 | F | X-B | N-[(6-hydroxy-2-isopropyl-pyrimidin-4-yl)methoxy]- | CH₂ | B | 0.995 | 415.8 |
| | | | C-methyl-carbonimidoyl | | | | |
| F-49 | F | X-B | N-[(2-fluorophenyl)methoxy]-C-methyl- | CH₂ | B | 1.238 | 373.7 |
| | | | carbonimidoyl | | | | |
| F-50 | F | X-B | C-methyl-N-[[5-(trifluoromethyl)-2- | CH₂ | B | 1.208 | 424.8 |
| | | | pyridyl]methoxy]carbonimidoyl | | | | |
| F-51 | Cl | X-A | N-[(3,4-dichlorophenyl)methoxy]-C-methyl- | NH | B | 1.362 | 429.4 |
| | | | carbonimidoyl | | | | |
| F-52 | CN | X-B | [1-(2-chloro-4-iodo-phenyl)pyrazol-3-yl]oxymethyl | CH₂ | B | 1.272 | 547.8 |
| F-53 | CN | X-B | [1-(2,4-difluorophenyl)pyrazol-3-yl]oxymethyl | CH₂ | B | 1.149 | 423.9 |
| F-54 | CN | X-B | [1-(2,4-dichlorophenyl)-4-methyl-pyrazol-3- | CH₂ | B | 1.313 | 469.9 |
| | | | yl]oxymethyl | | | | |
| F-55 | CN | X-B | [1-(2,4-dichlorophenyl)-1,2,4-triazol-3-yl]oxymethyl | CH₂ | B | 1.108 | 456.8 |
| F-56 | CN | X-B | [1-(4-ethylphenyl)pyrazol-3-yl]oxymethyl | CH₂ | B | 1.232 | 415.9 |
| F-57 | CN | X-B | [1-(3,5-dichlorophenyl)-1,2,4-triazol-3-yl]oxymethyl | CH₂ | B | 1.184 | 458.6 |
| F-58 | CN | X-B | [4-[N-ethoxy-C- | CH₂ | B | 1.269 | 460.9 |
| | | | (trifluoromethyl)carbonimidoyl]phenoxy]methyl | | | | |
| F-59 | CN | X-B | (3,5-difluorophenoxy)methyl | CH₂ | B | 1.120 | 357.8 |
| F-60 | CN | X-B | [4-(4-bromophenyl)phenoxy]methyl | CH₂ | B | 1.313 | 477.7 |
| F-61 | CN | X-B | [1-(4-chlorophenyl)pyrazol-3-yl]oxymethyl | CH₂ | B | 1.194 | 421.9 |
| F-62 | F | X-A | [1-(2-chloro-4-methyl-phenyl)pyrazol-3-yl]oxymethyl | CH₂ | B | 1.207 | 415.8 |
| F-63 | F | X-A | [1-(4-bromo-3-chloro-phenyl)pyrazol-3-yl]oxymethyl | CH₂ | B | 1.291 | 480.8 |
| F-64 | F | X-A | [1-[5-(trifluoromethyl)-2-pyridyl]pyrazol-3- | CH₂ | B | 1.242 | 436.8 |
| | | | yl]oxymethyl | | | | |
| F-65 | F | X-A | [5-methyl-1-[5-(trifluoromethyl)-2-pyridyl]-1,2,4- | CH₂ | B | 1.169 | 451.8 |
| | | | triazol-3-yl]oxymethyl | | | | |
| F-66 | F | X-A | [1-[2-chloro-4-(trifluoromethyl)phenyl]-1,2,4-triazol- | CH₂ | B | 1.167 | 470.8 |
| | | | 3-yl]oxymethyl | | | | |
| F-67 | F | X-A | [4-(4-chlorophenyl)thiazol-2-yl]oxymethyl | CH₂ | B | 1.291 | 418.8 |
| F-68 | F | X-A | (6-chloro-2-pyridyl)oxymethyl | CH₂ | B | 1.108 | 336.7 |
| F-69 | F | X-A | [6-[3-(trifluoromethyl)phenoxy]-2-pyridyl]oxymethyl | CH₂ | B | 1.310 | 462.9 |
| F-70 | F | X-A | [2-cyclopropyl-6-(trifluoromethyl)pyrimidin-4- | CH₂ | B | 1.220 | 411.8 |
| | | | yl]oxymethyl | | | | |
| F-72 | F | X-A | (6-methylpyrimidin-4-yl)oxymethyl | CH₂ | B | 0.756 | 317.8 |
| F-73 | F | X-A | [4-(N-butoxy-C-methyl-carbonimidoyl)-2-cyano- | CH₂ | B | 1.331 | 439.8 |
| | | | phenoxy]methyl | | | | |
| F-74 | F | X-A | [4-(3-chloroallyloxyiminomethyl)phenoxy]methyl | CH₂ | B | 1.244 | 418.8 |

HPLC MS=high performance liquid chromatography-coupled mass spectrometry; HPLC methods:
A) Luna-C18(2) 2.00×50 mm, 5 μm; mobile phase: A: water+0.056% tri-fluoroacetic acid (TFA); B: acetonitrile+0056% TFA; gradient: 10-80% B in 4.00 minutes; 80% B 0.90 min; flow: 0.8 ml/min at 40° C. MS: quadrupole electrospray ionization (positive mode).
B) Phenomenex Kinetex 1.7 μm XB-C18 100A; 50×2.1 mm; mobile phase: A: water+0.1% tri-fluoroacetic acid (TFA); B: acetonitrile+0.1% TFA; gradient: 5-100% B in 1.50 minutes; 100% B 0.20 min; flow: 0.8-1.0 ml/min in 1.50 minutes at 60° C. MS: quadrupole electrospray ionization, 80 V (positive mode).

### Example F-8

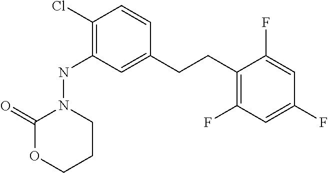 [SMILES: O=C1OCCCN1Nc1cc(CCc2c(F)cc(F)cc2F)ccc1Cl]

### 3-(5-bromo-2-chloro-anilino)-1,3-oxazinan-2-one

To a solution of (5-bromo-2-chloro-phenyl)hydrazine (22.0 g, 100 mmol) and 3-chloropropyl carbonochloridate (15.6 g, 100 mmol) in 300 mL tetrahydrofurane (THF) at 0° C. was added dropwise triethylamine (20 mL). The reaction mixture was allowed to slowly warm to room temperature and stirred for 2 hours. The reaction mixture was filtered; the obtained solid was washed with water, and then dried under vacuum to give 18.2 g of a white solid. This solid was dissolved in 100 mL dry THF, K₂CO₃ (14.7 g, 106 mmol) was added and the mixture was stirred at room temperature overnight. The mixture was filtered and water was added. Extraction with ethyl acetate, drying over Na₂SO₄, evaporation of the solvent and purification by column chromatography gave 3-(5-bromo-2-chloro-anilino)-1,3-oxazinan-2-one (12.4 g, 77%) as an off-white solid.

### 3-(2-chloro-5-ethynyl-anilino)-1,3-oxazinan-2-one

3-(5-bromo-2-chloro-anilino)-1,3-oxazinan-2-one (6.2 g, 20.0 mmol) was dissolved in dry triethylamine (50 mL) and trimethylsilylacetylene (2.0 g, 20.0 mmol) was added, followed by Pd(PPh₃)₂Cl₂ (150 mg, 0.2 mmol) and Cul (38.2 mg, 0.2 mmol). The mixture was heated to 60° C. and stirred under nitrogen overnight. The reaction mixture was cooled to room temperature, extracted with ethyl acetate and water and the organic layer was dried over Na₂SO₄ and concentrated in vacuum. Purification by column chromatography gave 3-(2-chloro-5-ethynyl-anilino)-1,3-oxazinan-2-one (4.70 g, 74%) as a yellow oil, which was immediately dissolved in CH₃OH (30 mL). K₂CO₃ (2.02 g, 14.6 mmol) was added and the mixture was stirred for 2 hours at room temperature. Filtration, extraction with ethyl acetate and water, drying of the organic layer over Na₂SO₄ and evaporation gave the crude product. Purification by column chromatography yielded in 3-(2-chloro-5-ethynyl-anilino)-1,3-oxazinan-2-one (2.1 g, 42%) as a pale white solid.

### 3-[2-chloro-5-[2-(2,4,6-trifluorophenyl)ethynyl]anilino]-1,3-oxazinan-2-one

3-(2-chloro-5-ethynyl-anilino)-1,3-oxazinan-2-one (2.1 g, 8.4 mmol) was dissolved in dry triethylamine (25 mL) and 2-bromo-1,3,5-trifluoro-benzene (1.76 g, 8.4 mmol) was added, followed by Pd(PPh₃)₂Cl₂ (115 mg, 0.1 mol) and Cul (19.1 mg, 0.1 mmol). The mixture was heated to 60° C. and stirred under nitrogen overnight. After cooling to room temperature water was added and the mixture was extracted with ethyl acetate. Drying over Na₂SO₄, removing of the solvent under reduced pressure and column chromatography gave 3-[2-chloro-5-[2-(2,4,6-trifluorophenyl)ethynyl]anilino]-1,3-oxazinan-2-one (250 mg, 78%).

### 3-[2-chloro-5-[2-(2,4,6-trifluorophenyl)ethyl]anilino]-1,3-oxazinan-2-one

3-[2-Chloro-5-[2-(2,4,6-trifluorophenyl)ethynyl]anilino]-1,3-oxazinan-2-one (250 mg, 0.66 mmol) was dissolved in dry THF (10 mL) and PtO₂ (100 mg) was added. The mixture was stirred under a hydrogen atmosphere for 2 hours at room temperature. Filtration and concentration in vacuum gave the crude product which was purified by prep. HPLC to give 3-[2-chloro-5-[2-(2,4,6-trifluorophenyl)ethyl]anilino]-1,3-oxazinan-2-one (110 mg, 45%) as a yellow oil.

### Example F-43

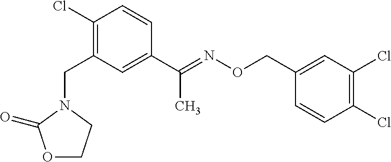 [SMILES: C/C(=N\OCc1ccc(Cl)c(Cl)c1)c1ccc(Cl)c(CN2CCOC2=O)c1]

### 4-bromo-2-(bromomethyl)-1-chloro-benzene

To a solution of 4-bromo-1-chloro-2-methyl-benzene (30 g, 146 mmol) and benzoyl peroxide (0.71 g, 3 mmol) in acetonitrile (70 mL) was added N-bromosuccinimide (28.6 g, 161 mmol) at 90° C. and the mixture was stirred overnight. The solution was evaporated under reduced pressure and the crude material was purified onsilica gel to give 4-bromo-2-(bromomethyl)-1-chloro-benzene as a white solid (40.3 g, 95% yield).

### 3-[(5-bromo-2-chloro-phenyl)methyl]oxazolidin-2-one

A solution of oxazolidin-2-one (17.8 g, 204 mmol), NaH (8.16 g, 204 mmol) was stirred in CH₂Cl₂ (200 mL) at 0° C. for 1 hour. 4-bromo-2-(bromomethyl)-1-chloro-benzene (47.8 g, 170 mmol) was added and the mixture was stirred at room temperature overnight. Saturated ammonium chloride solution was added and the solution was diluted with 200 mL water. Extraction with tert-butyl methyl ether, combination of the organic layers, drying over Na₂SO₄ and concentrating in vacuum gave the crude product which was purified on silica gel to give 3-[(5-bromo-2-chloro-phenyl)methyl]oxazolidin-2-one as a yellow solid (38.0 g, yield 99%).

### 3-[(5-acetyl-2-chloro-phenyl)methyl]oxazolidin-2-one

To a solution of 3-[(5-bromo-2-chloro-phenyl)methyl]oxazolidin-2-one (15 g, 516 mmol) and tributyl(1-methoxyvinyl)stannane (19.8 g, 568 mmol) in dioxane (150 mL) Pd(PPh₃)₄ (3 g, 2.58 mmol) was added and the mixture was stirred at 85° C. for 10 hours. 3N HCl (20 mL) was added and it was stirred at room temperature for 30 minutes. The reaction mixture was concentrated in vacuum and the crude material was purified prep. HPLC to give 3-[(5-acetyl-2-chloro-phenyl)methyl]oxazolidin-2-one as yellow solid (5.4 g, 41% yield).

### 3-[[2-chloro-5-[(E)-N-[(3,4-dichlorophenyl)methoxy]-C-methyl-carbonimidoyl]phenyl]methyl]oxazolidin-2-one

O-[(3,4-Dichlorophenyl)methyl]hydroxylamine (114 mg, 593 μmol) was added to a solution of 3-[(5-acetyl-2-chloro-phenyl)methyl]oxazolidin-2-one (150 mg, 591 μmol) in ethanol (3 mL) and the solution was stirred at 90° C. overnight. After completion the solvent was removed under reduced pressure and the crude material was purified by prep. HPLC to yield in 3-[[2-chloro-5-[(E)-N-[(3,4-dichlorophenyl)methoxy]-C-methyl-carbonimidoyl]phenyl]methyl]oxazolidin-2-one (168 mg, 66%).

### II. Examples of the Action Against Harmful Fungi

The activity against phytopathogenic fungi could be demonstrated by the treatment of fungal spore suspensions and analysis of the growth in microplates using a robot system.

The tests were done in 96 well microtiter plates. The compounds were transferred as solutions in dimethylsulfoxide (DMSO) into empty plates, followed by a spore suspension of the fungus of interest in a nutrient solution. The compounds were tested either in a single dose or as serial dilution in 10 doses. Each plate contained 8 solvent control wells and 8 reference wells containing a known fungicide. The plates were incubated at 23° C. and 90% relative humidity. Fungal growth was assessed by measuring the optical density at 620 nm immediately after treatment and 10 times in intervals of 15 hours.

In order to calculate the activity of a compound on a given dose, the optical density values of each measurement of a compound is compared with those of the control and the reference, giving results from 0 to 1. The antifungal activity increases with increasing values. ED₅₀ values can be obtained from the dilution series.

A compound having an activity value ≧0.75 at 31 ppm or an ED₅₀ value ≦31 ppm is considered as fungicidal active.

The in-vitro activity of compounds F-1, F-2, F-3, F-5, F-6, F-7, F-9, F-10, F-11, F-12, F-16, F-17, F-19, F-22, F-26, F-27, F-28, F-29, F-30, F-31, F-36, F-38, F-39, F-40, F-41, F-47, F-49, F-51, F-52, F-53, F-54, F-55, F-58, F-59, F-63, F-64, F-67, F-70, and F-74 against four important phytopathogenic fungi was investigated and was observed as follows:

| fungus | Activity shown for compounds |
|---|---|
| Botrytis cinerea | F-1, F-7, F-9, F-16, F-22, F-28, F-31, F-38, |
| | F-39, F-40, F-41, F-47, F-49, F-52, F-54, F-58 |
| Phytophthera infestans | F-1, F-3, F-7, F-10, F-30, F-47, F-53, F-55, F-59 |
| Pyricularia oryzae | F-1, F-2, F-3, F-5, F-6, F-7, F-9, F-11, F-12, |
| | F-16, F-17, F-19, F-22, F-26, F-27, F-28, F-29, |
| | F-30, F-36, F-38, F-39, F-40, F-41, F-47, F-51, |
| | F-52, F-54, F-58, F-63, F-64, F-67, F-70, F-74 |
| Septoria tritici | F-1, F-3, F-7, F-17, F-39, F-47, F-51 |

1-13. (canceled)

14. A compound of formula I 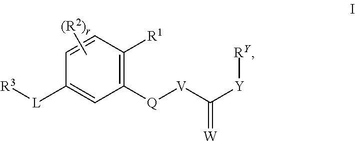
wherein:
R¹ is halogen, cyano, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₁-C₆-alkylthio, C₁-C₆-alkylsulfinyl, C₁-C₆-alkylsulfonyl, C₃-C₆-cycloalkyl or C₃-C₆-cycloalkyl-C₁-C₄-alkyl; wherein the aliphatic and alicyclic moieties of R¹ are unsubstituted or substituted by 1, 2, 3 or up to the maximum number of identical or different groups R^{1a}; wherein
R^{1a} is halogen, hydroxy, cyano, nitro, C₁-C₄-alkyl, C₁-C₄-alkoxy, C₃-C₆-cycloalkyl, C₁-C₄-haloalkyl or C₁-C₄-haloalkoxy;
R² is halogen, hydroxy, cyano, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl or C₃-C₆-cyclo-alkyl-C₁-C₄-alkyl; wherein the aliphatic and alicyclic moieties of R² are unsubstituted or substituted by 1, 2, 3 or up to the maximum number of identical or different groups R^{2a}; wherein
R^{2a} is halogen, hydroxy, cyano, nitro, C₁-C₄-alkyl, C₁-C₄-alkoxy, C₃-C₆-cycloalkyl, C₁-C₄-haloalkyl or C₁-C₄-haloalkoxy;
r is 0, 1, 2 or 3;
L is a direct bond or a divalent group selected from —OCH₂—, —CH₂—, —CH₂CH₂—, —O—, —CH₂—O—N═C(Z)—, —O—N═C(Z)—, —C(Z)═N—O—CH₂—, —CHZ—C(Z)═N—O—CH₂—, —O—N═C(Z)—C(Z)═N—O—CH₂— and —C(═O)—C(Z)═N—O—CH₂—; wherein the bond depicted on the left side of the divalent group L is attached to R³, and the bond depicted on the right side is attached to the phenyl ring; wherein
Z is independently selected from hydrogen, amino, C₁-C₄-alkyl, C₁-C₄-haloalkyl and C₁-C₆-alkoxyimino-C₁-C₄-alkyl;
R³ is phenyl or a 5- or 6-membered aromatic heterocycle, wherein the ring member atoms of the heterocycle include besides carbon atoms 1, 2, 3 or 4 heteroatoms independently selected from N, O and S as ring member atoms; wherein the cyclic groups R³ are unsubstituted or substituted by 1, 2, 3, 4 or up to the maximum possible number of identical or different groups R^{3a}; wherein
R^{3a} is amino, halogen, hydroxy, nitro, cyano, carboxyl, C₁-C₆-alkyl, C₂-C₆-alkenyl, C₂-C₆-alkynyl, C₁-C₆-alkoxy, C₃-C₆-cycloalkyl, C₃-C₆-cycloalkenyl, C₂-C₆-alkenyloxy, C₃-C₆-alkynyloxy, C₂-C₆-alkenyloxyimino-C₁-C₄-alkyl, C₃-C₆-alkynyloxyimino-C₁-C₄-alkyl, C₁-C₆-alkylamino, C(═O)—(C₁-C₆-alkyl), C(═O)—(C₁-C₆-alkoxy), phenyl, naphthyl or a 3- to 10-membered saturated, partially unsaturated or aromatic mono- or bicyclic heterocycle, wherein the ring member atoms of the heterocycle include besides carbon atoms 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the aforementioned phenyl and heterocycle groups R^{3a} are attached to R³ via a direct bond, an oxygen or sulfur atom, the latter two atoms forming a linker between said residues; and wherein the aliphatic or cyclic groups R^{3a} are unsubstituted or substituted by 1, 2 or 3 or up to the maximum possible number of identical or different groups R^{3b}; wherein
R^{3b} is halogen, hydroxy, nitro, cyano, carboxyl, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₂-C₆-alkenyloxyimino-C₁-C₄-alkyl, C₃-C₆-alkynyloxyimino-C₁-C₄-alkyl, C₁-C₆-alkylthio, C₁-C₆-alkylsulfinyl, C₁-C₆-alkylsulfonyl, C₃-C₆-cycloalkyl, C₃-C₆-cycloalkenyl, phenyl or a 5- or 6-membered saturated, partially unsaturated or aromatic heterocycle; wherein the ring member atoms of the heterocycle include besides carbon atoms 1, 2 or 3 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the aforementioned cyclic groups R^{3b} are attached to R^{3a} via a direct bond, an oxygen or sulfur atom, the latter two atoms forming a linker between said residues; and wherein the aliphatic or cyclic groups R^{3b} are unsubstituted or substituted by 1, 2 or 3 or up to the maximum possible number of identical or different groups selected from halogen, C₁-C₆-alkyl and C₁-C₆-haloalkyl;
Q is a divalent group selected from —O—, —S—, —(NQ^{a})-, —(CQ^{b}Q^{c})-, —C(═N—O-Q^{a})-, —C(═O)— and —C(═S)—; wherein
Q^{a} is hydrogen, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl, phenyl-C₁-C₄-alkyl, heteroaryl-C₁-C₄-alkyl or C₃-C₆-cycloalkyl-C₁-C₄-alkyl; wherein the aliphatic, alicyclic and aromatic moieties of Q^{a} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy;
Q^{b}, Q^{c} are independently selected from hydrogen, halogen, cyano, C₁-C₆-alkyl, C₁-C₆-alkoxy, C₂-C₆-alkenyl, C₂-C₆-alkenyloxy, C₂-C₆-alkynyloxy, C₂-C₆-alkynyl, C₃-C₆-cycloalkyl and C₃-C₆-cycloalkyl-C₁-C₄-alkyl; wherein the aliphatic and alicyclic moieties of Q^{b} and/or Q^{c} are unsubstituted or substituted by 1, 2, 3 or up to the maximum number of identical or different groups selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy; or
Q^{b} and Q^{c} together with the carbon atom to which they are bound form a saturated or partially unsaturated 3-, 4-, 5-, 6- or 7-membered carbocycle or a saturated or partially unsaturated 3-, 4-, 5-, 6- or 7-membered heterocycle, wherein the heterocycle includes beside carbon atoms 1, 2, 3 or 4 heteroatoms independently selected from N, O and S as ring member atoms; and wherein 1 or 2 carbon ring member atoms of the carbo- and heterocycle may be replaced by 1 or 2 groups independently selected from C(═O) and C(═S); and wherein the carbo- and heterocycle are unsubstituted or substituted by 1, 2, 3 or 4 identical or different groups independently selected from halogen, hydroxy, cyano, nitro, C₁-C₄-alkyl, C₁-C₄-alkoxy, C₃-C₆-cycloalkyl, C₁-C₄-haloalkyl and C₁-C₄-haloalkoxy;
V is a divalent group —NR^{V}—;
W is O or S;
Y is a divalent group selected from —O—, —S— and —NY^{a}—; wherein
Y^{a} is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, phenyl-C₁-C₄-alkyl, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy;
and wherein
R^{V} and R^{Y}, together with the group —(N—C(═W)—Y)—, form a 5-, 6- or 7-membered saturated heterocycle; wherein the heterocycle includes beside carbon atoms 1, 2, 3 or 4 heteroatoms independently selected from N, O and S as ring member atoms; and wherein 1 or 2 carbon ring member atoms of the heterocycle may be replaced by 1 or 2 groups independently selected from C(═O) and C(═S); and wherein the heterocycle is unsubstituted or substituted by 1, 2, 3 or 4 identical or different groups R^{X}; wherein
R^{X} is hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, C₁-C₆-alkylamino, C₁-C₆-dialkylamino or C₃-C₆-cycloalkylamino; wherein the aliphatic and alicyclic moieties of R^{X} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy;
or an N-oxide or an agriculturally acceptable salt thereof, with the proviso that 3-[[5-[4-(dichloromethyl)thiazol-2-yl]-2-fluoro-phenyl]methyl]oxazolidin-2-one be excluded.

15. The compound of claim 14, wherein R¹ is F, Cl, Br, cyano, CH₃ or OCH₃.

16. The compound of claim 14, wherein L is —OCH₂—, wherein the bond depicted on the left side of the group —OCH₂— is attached to R³ and the bond depicted on the right side is attached to the phenyl ring; or L is —CH₂—O—N═C(Z)— or —O—N═C(Z)—.

17. The compound of claim 14, wherein the group R³ is a pyridinyl ring, which is attached to L in 2-position and which is further unsubstituted or substituted by 1, 2 or 3 identical or different groups R^{3a}; wherein
R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, SCF₃, SCHF₂, cyano, Cl, F or Br.

18. The compound of claim 14, wherein W is O.

19. The compound of claim 14, wherein Y^{a} and Q^{a} are independently selected from hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy.

20. The compound of claim 14, wherein Y^{a} and R^{x} are independently selected from hydrogen, CH₃, CH₂CH₃, CF₃, CHF₂, OCF₃, OCHF₂, OCF₃, F, Cl, and Br.

21. The compound of claim 14, wherein Q is —CH₂—, —NH— or —NCH₃—.

22. An agrochemical composition which comprises an auxiliary and at least one compound of formula I, or an N-oxide or an agriculturally acceptable salt thereof, according to claim 14.

23. An agrochemical composition of claim 22 comprising at least one further active substance.

24. A method for combating phytopathogenic harmful fungi, which process comprises treating the fungi or the materials, plants, the soil or seeds to be protected against fungal attack, with an effective amount of at least one compound of formula I or an N-oxide or an agriculturally acceptable salt thereof, as defined in claim 14.

25. Seed treated with the compound of claim 14, or an N-oxide or an agriculturally acceptable salt thereof, in an amount of from 0.1 g to 10 kg per 100 kg of seed.

26. The method of claim 24, wherein, in the compound of formula I, R¹ is F, Cl, Br, cyano, CH₃ or OCH₃.

27. The method of claim 24, wherein, in the compound of formula I, L is —OCH₂—, wherein the bond depicted on the left side of the group —OCH₂— is attached to R³ and the bond depicted on the right side is attached to the phenyl ring; or L is —CH₂—O—N═C(Z)— or —O—N═C(Z)—.

28. The method of claim 24, wherein, in the compound of formula I, the group R³ is a pyridinyl ring, which is attached to L in 2-position and which is further unsubstituted or substituted by 1, 2 or 3 identical or different groups R^{3a}; wherein
R^{3a} is methoxyimino-C₁-C₄-alkyl, ethoxyimino-C₁-C₄-alkyl, CH₃, CH₂CH₃, OCH₃, OCH₂CH₃, CF₃, CHF₂, OCF₃, OCHF₂, SCF₃, SCHF₂, cyano, Cl, F or Br.

29. The method of claim 24, wherein, in the compound of formula I, W is O.

30. The method of claim 24, wherein, in the compound of formula I, Y^{a} and Q^{a} are independently selected from hydrogen, C₁-C₆-alkyl, C₁-C₆-alkoxy, C₂-C₆-alkenyl, C₂-C₆-alkenyloxy, C₂-C₆-alkynyl, C₃-C₆-alkynyloxy, C₃-C₆-cycloalkyl, C₃-C₆-cycloalkoxy, heteroaryl-C₁-C₄-alkyl, (C═O)—(C₁-C₆-alkyl) or (C═O)—(C₁-C₆-alkoxy); wherein the aliphatic, alicyclic and aromatic moieties of Y^{a} are unsubstituted or substituted by 1, 2, 3 or up to the maximum number of identical or different groups selected from hydrogen, halogen, cyano, nitro, C₁-C₄-alkyl, C₁-C₄-alkoxy, C₃-C₆-cycloalkyl, C₁-C₄-haloalkyl and C₁-C₄-haloalkoxy.

31. The method of claim 24, wherein, in the compound of formula I, Y^{a} and R^{x} are independently selected from hydrogen, CH₃, CH₂CH₃, CF₃, CHF₂, OCF₃, OCHF₂, OCF₃, F, Cl, and Br.

32. The method of claim 24, wherein, in the compound of formula I, Q is —CH₂—, —NH— or —NCH₃—.
